(19) United States
(12) Patent Application Publication
(10) Pub. No.: US 2023/0013145 A1
(43) Pub. Date: Jan. 19, 2023

(54) AAV CAPSID-PROMOTER INTERACTIONS AND CELL SELECTIVE GENE EXPRESSION

(71) Applicant: The University of North Carolina at Chapel Hill, Chapel Hill, NC (US)

(72) Inventors: Thomas McCown, Carrboro, NC (US); Sara Powell, Chapel Hill, NC (US); Richard Jude Samulski, Hillsborough, NC (US)

(21) Appl. No.: 17/904,433

(22) PCT Filed: Feb. 17, 2021
(86) PCT No.: PCT/US2021/018280

§ 371 Date: Aug. 17, 2022

Related U.S. Application Data
(60) Provisional application No. 62/977,838, filed on Feb. 18, 2020.
(60) Provisional application No. 63/023,003, filed on May 11, 2020.

(51) Int. Cl. A61K 48/00; C12N 15/86; C12N 5/079

(52) U.S. Cl. CPC A61K 48/0025; C12N 15/86; C12N 5/0618; C12N 2750/14122; C12N 2750/14142; C12N 2750/14145

(57) ABSTRACT

Disclosed herein are methods and compositions for gene therapy, in particular, methods and compositions related to alterations in a capsid protein sequence for altering permissiveness of a promoter within a cell when the promoter and the capsid protein are present within the cell and the capsid protein and the promoter are in the context of a recombinant adeno-associated virus (AAV) particle.

### STATEMENT OF PRIORITY

This application claims the benefit of U.S. Provisional Application Ser. No. 62/977,838, filed Feb. 18, 2020, and U.S. Provisional Application Ser. No. 63/023,003, filed May 11, 2020, the entire contents of each of which are incorporated by reference herein.

### GOVERNMENT SUPPORT

This invention was made with government support under Grant Number NS082289 awarded by the National Institutes of Health. The government has certain rights in the invention.

### FIELD OF THE INVENTION

This invention relates to methods and compositions for gene therapy. In particular, the invention relates to methods and compositions for altering permissiveness of a promoter within a cell when the promoter and the capsid protein are present within the cell and the capsid protein and the promoter are in the context of a recombinant adeno-associated virus particle.

### BACKGROUND OF THE INVENTION

Given highly favorable in vivo properties, adeno-associated virus (AAV) vectors have attained a prominent role in preclinical and clinical gene therapy studies. Importantly, for many applications successful outcomes depend upon achieving specific properties of cellular transduction and gene expression. One means to achieve selective vector properties involves manipulation of the AAV capsid structure, based upon the role of the capsid in cellular binding, endosome escape and nuclear localization. The AAV capsid gene encodes three structural proteins VP1, VP2 and VP3 that assemble into the capsid in a 1:1:10 ratio. VP1 and VP2 have unique N terminal sequences that are not in VP3, while the VP3 sequence (C terminal) is shared among all VP proteins (Buller et al., J. Virol. 25:331 (1978); Johnson et al., J. Virol. 8:860 (1971); Rose et al., J. Virol. 8:766 (1971)). Manipulation of the capsid has focused upon the VP3 sequence primarily through the use of rationale mutagenesis or capsid DNA shuffling. In the case of capsid DNA shuffling, manipulating AAV capsid DNA by DNA shuffling or error prone PCR creates an AAV capsid library which contains substantial diversity (Asokan et al., Mol. Ther. 20:699 (2012)). Subsequent administration of this library either in vitro or in vivo provides the means to rescue novel capsids that fulfill the specific selection pressures exerted by directed evolution parameters. For example, novel capsids have been generated that efficiently transduce neural stem cells (Jang et al., Mol. Ther. 19:667 (2011)) or oligodendrocytes (Powell et al., Gene Ther. 23:807 (2016)).

Although capsid engineering has focused upon manipulating VP3, recent studies suggest that the capsid proteins, including VP1 and VP2, can influence cellular gene expression. Johnson et al. (J. Virol. 84:8888 (2010)) established that mutations in the AAV2 VP2 capsid significantly altered gene expression in vitro. Similarly, Aydemir et al. (J. Virol. 90:7196 (2016)) reported that single point mutants in the “dead zone” on the AAV2 capsid surface resulted in packaged AAV vectors that exhibited normal receptor binding, intracellular trafficking and proper uncoating in the nucleus, but failed to transcribe the gene. These studies discussed that AAV capsids might exert significant influence on cellular gene expression. However, there is still a need for discovering new ways to modify transgene expression from AAV vectors to overcome current shortcomings in the art.

### SUMMARY OF THE INVENTION

Aspects of the invention relate to the discovery of a previously unknown role of the AAV capsid protein in promoter activity in different cell types. Studies suggested that specific regions of capsid proteins can alter in vivo cellular gene expression of the viral genome from the operably linked promoter, and that this alteration impacts the cell types in which expression occurs. We have identified a previously unknown interaction between the AAV capsid and promoters that can provide selective cellular gene expression in vivo, and further teach how this interaction can be manipulated (e.g., by modification of the capsid protein) to influence cell type expression from the AAV vector.

Accordingly, one aspect of the present invention relates to an AAV capsid protein or a derivative thereof comprising at least a portion of an AAV VP1/VP2 boundary, the capsid protein comprising an amino acid sequence modification at one or more amino acids within the VP1/VP2 boundary that alters permissiveness of a promoter within a cell when the promoter and the capsid protein are present within the cell, and wherein the capsid protein and the promoter are in the context of a recombinant AAV particle.

A further aspect of the invention relates to a method for altering expression of a transgene operably linked to a promoter and delivered to a cell by a rAAV vector comprising modifying the amino acid sequence of at least one amino acid within the VP1/VP2 boundary of a capsid protein or derivative thereof of the rAAV vector, wherein the amino acid sequence modification alters the permissiveness of the promoter within the cell.

An additional aspect of the invention relates to a nucleic acid encoding an AAV capsid protein or derivative thereof comprising at least a portion of an AAV VP1/VP2 boundary, the capsid protein comprising an amino acid sequence modification at one or more amino acids within the VP1/VP2 boundary that alters permissiveness of a promoter within a cell when the promoter and the capsid protein are present within the cell, and wherein the capsid protein and the promoter are in the context of a recombinant AAV particle.

Another aspect of the invention relates to vectors, cells, virus particles, and AAV particles comprising the nucleic acid of the invention.

A further aspect of the invention relates to a method of producing a recombinant AAV particle comprising an AAV capsid, the method comprising: providing a cell in vitro with a nucleic acid according to the invention, an AAV rep coding sequence, an AAV vector genome comprising a promoter operably linked to a heterologous nucleic acid, and helper functions for generating a productive AAV infection; and allowing assembly of the recombinant AAV particle comprising the AAV capsid and encapsidating the AAV vector genome.

An additional aspect of the invention relates to a pharmaceutical formulation comprising the AAV capsid protein or derivative thereof, nucleic acid, virus particle, or AAV particle of the invention in a pharmaceutically acceptable carrier.

Another aspect of the invention relates to a method of delivering a nucleic acid of interest to a central nervous system (CNS) cell, the method comprising contacting the cell with the AAV particle of the invention.

A further aspect of the invention relates to a method of delivering a nucleic acid of interest to a cell in a mammalian subject, the method comprising administering an effective amount of the AAV particle or the pharmaceutical formulation of the invention to a mammalian subject, thereby delivering the nucleic acid of interest to a cell in the mammalian subject. In some embodiments, the cell is in the CNS of the subject.

An additional aspect of the invention relates to a method of treating a disorder in a mammalian subject in need thereof, wherein the disorder is treatable by expressing a therapeutic product in a cell (e.g., a CNS cell) of the subject, the method comprising administering a therapeutically effective amount of the AAV particle or the pharmaceutical formulation of the invention to a mammalian subject, wherein the product is expressed, thereby treating the disorder.

Another aspect of the invention relates to a method of altering expression of a heterologous polynucleotide present in an AAV vector in cells of a subject, comprising preparing the AAV vector with the AAV capsid protein or derivative thereof of the invention, and administering the AAV vector to the subject to thereby contact the cells of the subject. In some embodiments, the cells are in the CNS of the subject.

These and other aspects of the invention are set forth in more detail in the description of the invention below.

### BRIEF DESCRIPTION OF THE DRAWINGS

FIGS. 1A-1B show diagrams of the components of two constitutive promoters CBA and CBh used to drive reporter gene expression. 1A shows the shared elements of the CBA (1.6 kb) and CBh (0.8 kb) promoters: CMV Early promoter, chicken beta-actin promoter, and chicken beta-actin intron. The CBh promoter has a truncated chicken beta-actin intron and an MVM intron (minute virus of mouse). 1B provides diagrams of the AAV transgenes constructed to directly compare the CBA and CBh promoters with the identical backbone, AAV2 ITRs, mCherry transgene, DNA stuffer to increase transgene size and human growth hormone poly A. The transgenes are single stranded.

FIG. 2 shows a cresyl violet nissl stained section through the rat striatum where the oval outline indicates the range of AAV vector infusions.

FIGS. 3A-3J′ show representative confocal images of AAV2 and AAV9 with mCherry expression driven by a CBA or CBh promoter. Vectors were infused directly into the rat striatum at equal titers and equal volumes. mCherry transgene expression was compared with a neuronal cell marker (NeuN) and an oligodendrocyte marker (Olig2), and co-localization was quantified. Representative confocal images of AAV2-CBA-mCherry (N=4) illustrate NeuN localization (A-D) and Olig2 localization (E-H) with subsequent quantification over several images (I). Representative confocal images of AAV2-CBh-mCherry (N=6) illustrate NeuN localization (J-L) and Olig2 localization (N-P) with subsequent quantification over several images (R). Representative confocal images of AAV9-CBA-mCherry (n=6) illustrate NeuN localization (S-V) and Olig2 localization (W-Z) with subsequent quantification over several images (A′). Representative confocal images of AAV9-CBh-mCherry (N=6) illustrate NeuN localization (B′-E′) and Olig2 localization (F′-I′) with subsequent quantification over several images (J′). Scale bars, 50 μm. The white box indicates the zoomed in portion of the image that is shown to right of the image.

FIG. 4 shows alignment of AAV2 and AAV9 VP1 capsid residues surrounding the site of amino acid insertions (SEQ ID NOS:1-4). The entire amino acid sequence shown is a region surrounding the specific position within VP1 that VP2 amino acid sequence begins. In one embodiment of the invention, the region shown surrounding that specific position is referred to herein as the “VP1/VP2 boundary” or “junction region” (also indicated as the region of VP1/2 intersection in Example 1). This boundary includes the sequences unique to VP1 and the initial region of VP1/VP2 overlap, for AAV2 (top sequence shown) and AAV9 (bottom sequence shown). The arrow originates at the specific location of the intersection of the VP1 and VP2 amino acid sequence, (referred to herein as the VP1/VP2 junction), which falls between the C-terminal most end of the sequence that is unique to the VP1 protein and the beginning of the shared VP1 and VP2 amino acid sequence. The amino acid directly to the right of the arrow is a.a. 138 of VP1, and also corresponds to amino acid 1 (the N-terminus) of the VP2 protein. As such the VP1/VP2 junction is located between Lys137 and Thr138 of VP1, for both AAV2 and AAV9. Matching residues are gray while differing residues are black.

FIGS. 5A-5R show representative confocal images of AAV2EU-CBA and AAV9EU-CBA with mCherry expression driven by a CBA promoter. Vectors were infused directly into the rat striatum at equal titers and equal volumes. mCherry transgene expression was compared with a neuronal cell marker (NeuN) and an oligodendrocyte marker (Olig2), and co-localization was quantified. Representative confocal images of AAV2EU-CBA-mCherry (N=2) compared with NeuN localization (A-D), Olig2 localization (E-H), and the quantification over several images (I). Representative confocal images of AAV9EU-CBA-mCherry (N=5) compared with NeuN localization (J-M), Olig2 localization (N-Q), and the quantification over several images (R). Scale bars, 50 μm. The white box indicates the zoomed in portion of the image that is shown to right of the image.

FIGS. 6A-6W show representative confocal images of AAV2AU-CBA and AAV9AU-CBA with mCherry expression driven by a CBA promoter. Vectors were infused directly into the rat striatum at equal titers and equal volumes. mCherry transgene expression was compared with a neuronal cell marker (NeuN) and an oligodendrocyte marker (Olig2), and co-localization was quantified. Representative confocal images of AAV2AU-CBA-mCherry (N=2) compared with NeuN localization (A-D), Olig2 localization (E-H), and the quantification over several images (I). Representative confocal images of AAV9AU-CBA-mCherry (N=2) compared with NeuN localization (J-M), Olig2 localization (N-Q), and the quantification over several images (R). Representative confocal images of AAV9AU-CBh-mCherry (N=2) compared with NeuN localization (S-V) and the quantification over several images (W). Scale bars, 50 μm. The white box indicates the zoomed in portion of the image that is shown to right of the image.

FIGS. 7A-7F show representative confocal images of AAV9-MBP-GFP (N=2) and AAV9AU-MBP-GFP (N=2) gene expression in the rat striatum. Scale bars, 50 μm.

FIGS. 8A-8F show representative confocal images of self-complementary AAV9-CBh-GFP (N=2) gene expression in the rat striatum. The asterisks indicate co-localization of GFP and NeuN containing cells. Scale bars, 50 μm.

FIG. 9 shows AAV9 capsid interaction with the JeTI promoter.

FIG. 10 shows AAV8 CAB versus Cbh transduction in the rat striatum.

FIGS. 11A-11L show confocal images of AAV9-hSyn and AAV9AU-hSyn mediated transduction in the rat striatum. A-C show that AAV9-JetI-GFP gene expression primarily co-localizes with the oligodendrocyte marker, Olig2 while D-F illustrate the general lack of GFP co-localization with the neuronal marker, NeuN. In contrast, G-I illustrate that AAV9AU-JetI-GFP gene expression exhibits substantial co-localization with NeuN, but little co-localization with Olig2. Scale bars equal 20 microns.

FIG. 12 shows the total number of GFP-Olig2 positive cells or GFP-NeuN positive cells in the rat striatum using either AAV9 or AAV9AU vectors where gene expression was driven by the JetI synthetic promoter. * denotes a significant difference between the AAV9 and AAV9AU neurons (t<0.01). No difference was found for the number of GFP-Olig2 cells between AAV9 and AAV9AU.

FIGS. 13A-13F show AAV9-JetI-GFP (A-C) or AAV9AU-JetI-GFP (D-F) vectors do not exhibit GFP gene expression in GFAP positive astrocytes in the rat striatum.

Abbreviations for FIGS. 14-19: II—Oculomotor Nucleus; IV—Trochlear Nucleus; ac—Anterior Commissure; AM—Anteromedian Thalamic Nucleus; Amy—Amygdala; asp—Arcuate Spur; BIC—Brachium of the Inferior Colliculus; C—Caudate; CA—Cerebral Aqueduct; cc—Corpus Callosum; Cd—Caudate Nucleus; CG—Cingulate Gyms; Cl—Claustrum; cs—Central Sulcus; D—Dentate Nucleus; DR—Dorsal Raphe; F—Fastigial Nucleus; FEF—Frontal Eye Field (Area 8); GPe—External Segment of the Globus Pallidus; GPi—Internal Segment of the Globus Pallidus; Hc—Hippocampus; I—Interposed Nuclei; is—Inferior Arcuate Sulcus; ic—Internal Capsule; IC—Inferior Colliculus; its—Inferior Temporal Sulcus; ll—Lateral Lemniscus; LGN—Lateral Geniculate Nucleus; is—Lateral Sulcus; LV—Lateral Ventricle; mcp—Middle Cerebellar Peduncle; MD—Mediodorsal Thalamic Nucleus; ml—Medial Lemniscus; mlf—medial longitudinal fasciculus; MR—Median Raphe Nucleus; ot—Optic Tract; ox—Optic Chiasm; P—Putamen; PAG—Periaqueductal Gray; PGr—Pontine Gray; py—Pyramids; RN—Red Nucleus; RTP—Reticulotegmental Pontine Nucleus; sa—Superior Arcuate Sulcus; SC—Superior Colliculus; scp—Superior Cerebellar Peduncle; SEF—Supplementary Eye Field; SN—Substantia Nigra; ST—Subthalamic Nucleus; sts—Superior Temporal Sulcus; VPL—Ventroposterolateral Thalamic Nuclei; VPM—Ventroposteromedial Thalamic Nuclei; ZI—Zona Incerta.

FIGS. 14A-14E show chartings from a case illustrating neuronal labeling following injections of rAAV2-Retro-CAG-GFP injected into the frontal eye field of a rhesus macaque monkey. Red dots indicate the approximate location and density of neurons within example sections. Scale bar in B is true for all presented sections.

FIGS. 15A-15D show chartings from a case illustrating neuronal labeling following injections of rAAV2-Retro-hSyn-hChR2(H134R)-EYFP injected into the frontal eye field of a rhesus macaque monkey. Red dots indicate the approximate location and density of neurons within example sections. Scale bar in B is true for all presented sections.

FIGS. 16A-16L show photomicrographs from cases 1-3 illustrating neuronal labeling provided by AAV2-retro-CAG (primate cases 1, 2) or AAV2-retro-hSyn following injections into the frontal eye field (FEF). Asterisk indicates the location of needle tracts. Arrows indicate locations of individual neurons in photomicrographs where it may not be obvious to the observer.

FIGS. 17A-17F show chartings from two cases illustrating neuronal labeling following injections of rAAV2-Retro-CAG-GFP (Case 1, A-C) (Case 2, D-F) that were placed into the superior colliculus of two rhesus macaque monkeys. Red dots indicate the approximate location and density of neurons within example sections. Scale bar in A is true for all presented sections.

FIGS. 18A-18C show chartings from a case illustrating neuronal labeling following injections of rAAV2-Retro-hSyn-hChR2(H134R)-GFP (A-C) placed into the superior colliculus of a rhesus macaque monkey. Red dots indicate the approximate location and density of neurons within example sections. Scale bar in A is true for all presented sections.

FIGS. 19A-19I show photomicrographs from cases 4-6 illustrating neuronal labeling provided by AAV2-retro-CAG (cases 4, 5) or AAV2-retro-hSyn (case 6) following injections into the superior colliculus (SC). Arrows indicate locations of individual neurons in photomicrographs where it may not be obvious to the observer.

FIGS. 20A-20E show the amino acid sequence of capsid proteins of AAV 1-13, and alignments thereof (SEQ ID NOS:5-18).

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations, and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of recombinant and synthetic polypeptides, antibodies, or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, N.Y., 2012); F. M.

AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together.

The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “tropism” as used herein refers to entry of the virus into the cell, optionally and preferably followed by expression (e.g., transcription and, optionally, translation) of sequences carried by the viral genome in the cell, e.g., for a recombinant virus, expression of the heterologous nucleotide sequences(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of AAV, gene expression from the viral genome may be from a stably integrated provirus, from a non-integrated episome, as well as any other form in which the virus may take within the cell.

The term “tropism profile” refers to the pattern of transduction of one or more target cells, tissues and/or organs. Representative examples of chimeric AAV capsids have a tropism profile characterized by efficient transduction of cells of the central nervous system (CNS) with only low transduction of peripheral organs (see e.g., U.S. Pat. No. 9,636,370 McCown et al., and US patent publication 2017/0360960 Gray et al.). Vectors (e.g., virus vectors, e.g., AAV capsids) expressing specific tropism profiles may be referred to as “tropic” for their tropism profile, e.g., neuro-tropic, liver-tropic, etc.

The term “target cell” is used to refer to a cell that is infected by the viral vector described herein. In some embodiments, the “target cell” may refer to a cell type that is infected by the virus/viral vector and in which the regulatory elements on the heterologous nucleic acid effect promoter expression.

As used herein, the term “host cell” may refer to the packaging cell line in which a recombinant AAV is produced from a production plasmid. In the alternative, the term “host cell” may refer to any target cell which the rAAV particle infects, for in vitro assessment or in vivo delivery of a transgene. Such a cell is on occasion referred to as a target host cell.

As used herein, “transduction” of a cell by parvovirus or AAV refers to parvovirus/AAV-mediated transfer of genetic material into the cell. See, e.g., FIELDS et al., VIROLOGY, volume 2, chapter 69 (3d ed., Lippincott-Raven Publishers).

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide” or “nucleic acid,” may be of RNA, DNA or DNA-RNA hybrid (including both naturally occurring and non-naturally occurring nucleotides), but is preferably either a single or double stranded DNA sequence.

The term “regulatory element” refers to a genetic element which controls some aspect of the expression of nucleic acid sequences. For example, a promoter is a regulatory element which facilitates the initiation of transcription of an operably linked nucleic acid. A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “regulatory element” includes full-length promoter regions and functional (e.g., controls transcription or translation) segments of these regions. Other regulatory elements are splicing signals, polyadenylation signals, termination signals, etc. The region in a nucleic acid or polynucleotide in which one or more regulatory elements are found may be referred to as a “regulatory region.”

The term “operably linked” refers to an arrangement of elements wherein the complex of components are configured so as to perform its usual function (functional linkage). As used herein with respect to nucleic acids, the term “operably linked” refers to a functional linkage between two or more nucleic acids. For example, a promoter sequence may be described as being “operably linked” to a nucleic acid because the promoter sequences initiates and/or mediates transcription of the nucleic acid coding sequence. Thus, the term “operably linked” is intended to encompass any spacing or orientation of the promoter element and the nucleic acid of interest which allows for initiation of transcription of the nucleic acid of interest upon recognition of the promoter element by a transcription complex. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

A “therapeutic molecule” (e.g., a nucleic acid or polypeptide) is a molecule that can alleviate, reduce, prevent, delay and/or stabilize symptoms that result from an absence or defect in a protein in a cell or subject and/or is a molecule that otherwise confers a benefit to a subject e.g., anti-cancer effects or improvement in transplant survivability. Such therapeutic molecules may be encoded by a heterologous nucleic acid present in the viral vector described herein, and under the regulatory sequences that promote expression of the nucleic acid.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” or “purify” (or grammatical equivalents) a virus vector, it is meant that the virus vector is at least partially separated from at least some of the other components in the starting material.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “therapeutically effective amount” as used herein is an amount that is sufficient to provide some improvement or benefit to the subject. Alternatively stated, a “therapeutically effective amount” is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence with which the protein or nucleic acid in question is not found in relationship to in nature. Typically, the heterologous nucleic acid is a nucleic acid of interest that comprises open reading frame that encodes a polypeptide, and/or comprises a nontranslated RNA, each of which may be referred to herein as a “transgene”. The nucleic acid of interest may encode a therapeutic polypeptide or therapeutic RNA, or a diagnostic polypeptide or diagnostic RNA. The nucleic acid of interest/heterologous nucleic acid is often in the context of an expression cassette.

As the term is used herein, an “expression cassette” comprises the nucleic acid of interest operably linked/associated with appropriate regulatory element sequences (regulatory elements), for example, transcription/translation control signals, origins of replication, polyadenylation signals, internal ribosome entry sites (IRES), promoters, and/or enhancers, and the like. In preferred embodiments of the invention, the expression cassette comprises a (eukaryotic) promoter operably linked to the nucleic acid of interest, and optionally a (eukaryotic) transcription termination sequence. Typically, the expression cassette is flanked by viral packaging signals for viral vectors. For example, for an AAV viral vector, the packaging signals are at least one inverted terminal repeat which is located adjacent the expression cassette, optionally the 5′ inverted terminal repeat (ITR) and the 3′ ITR flank the expression cassette.

A “vector” refers to a compound used as a vehicle to carry foreign genetic material into another cell, where it can be replicated and/or expressed. A vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to as transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “viral vector” and “delivery vector” (and similar terms) generally refers to a vector that is derived from a virus. This could be an entire virus particle (an encapsidated genome) or could be a portion of the virus particle that functions as a nucleic acid delivery vehicle. At times, the term viral vector (e.g., AAV vector) refers to the viral genomic material engineered for delivery of a transgene contained within. As used herein, “viral genome” or “viral vector genome” (e.g., rAAV vector genome) refers such nucleic acid genomic material.

The viral vectors and viral genomes of the present invention can be made from, without limitation, parvoviruses, (e.g., dependoviruses) and particularly adeno-associated viruses. The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

The genus Dependovirus contains the adeno-associated viruses (AAV). AAV viruses are commonly referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralizing antibodies specific for any other AAV serotype. AAV serotypes include, without limitation, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, shrimp AAV, equine AAV, and ovine AAV. See, e.g., FIELDS et al., VIROLOGY, volume 2, chapter 69 (4th ed., Lippincott-Raven Publishers); and Table 1. As used herein, the term “adeno-associated virus” (AAV), includes, without limitation, any of these serotypes, and any other AAV now known or later discovered. See, e.g., FIELDS et al., VIROLOGY, volume 2, chapter 69 (4th ed., Lippincott-Raven Publishers). A number of relatively new AAV serotypes and clades have been identified (See, e.g., Gao et al., (2004) J. Virol. 78:6381; Moris et al., (2004) Virol. 33-:375; and Table 1).

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. This aspect of AAV makes viral vectors developed from AAV particularly desirable as gene delivery vectors for gene therapy.

The AAV genome used as a viral vector may, in theory, comprise the full genome of a naturally occurring AAV virus. For example, a vector comprising a full AAV genome may be used to prepare AAV virus in vitro. However, typically a recombinant AAV genome is generated from the AAV genome(s) of one or more AAV serotypes and used. Such manipulation is standard in the art and the present invention encompasses the use of any such derivative of an AAV genome which could be generated by applying techniques known in the art. Sequences in the recombinant AAV genome may be in a different order and configuration to that of a native AAV genome, or may be replaced with sequences from another virus or with chimeric genes composed of sequences from more than one virus. Such chimeric genes may be composed of sequences from two or more related viral proteins of different viral species.

TABLE 1

| | GenBank |
|---|---|
| AAV | Accession |
| Serotypes/Isolates | Number |
| Clonal Isolates | |
| Avian AAV ATCC | AY186198, |
| VR-865 | AY629583, |
| | NC_004828 |
| Avian AAV strain | NC_006263, |
| DA-1 | AY629583 |
| Bovine AAV | NC_005889, |
| | AY388617 |
| AAV4 | NC_001829 |
| AAV5 | AY18065, |
| | AF085716 |
| Rh34 | AY243001 |
| Rh33 | AY243002 |
| Rh32 | AY243003 |
| AAV10 | AY631965 |
| AAV11 | AY631966 |
| AAV12 | DQ813647 |
| AAV13 | EU285562 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73: 939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-:375-383; Mori et al., (2004) Virol. 330:375; Muramatsu et al., (1996) Virol. 221:208; Ruffing et al., (1994) J. Gen. Virol. 75:3385; Rutledge et al., (1998) J. Virol. 72:309; Schmidt et al., (2008) J. Virol. 82:8911; Shade et al., (1986) J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, Pa. (incorporated herein in its entirety).

The viral vectors of the invention can further be made from duplexed parvovirus particles as described in international patent publication WO 01/92551 (the disclosure of which is incorporated herein by reference in its entirety). Thus, in some embodiments, double stranded (duplex) genomes can be packaged within a vector particle.

A recombinant AAV (rAAV), as the term is used herein, refers to a viral vector generated from AAV through genetic manipulation to create an engineered product. Various rAAV have been generated and are typically referred to either as rAAV, rAAV vector, or AAV vector. The rAAV genome may comprise one or more heterologous nucleic acid sequences of interest, (e.g., encoding a marker or therapeutic polypeptide/RNA) for expression in a target/host cell. These are commonly referred to as a “transgene”. rAAV vectors generally require only the 145 base ITR in cis to generate virus (for replication and packaging). All other viral sequences (e.g., the coding sequences for the replication (rep) and/or capsid (cap)) are typically supplied in trans (Muzyczka (1992) Curr. Topics Microbiol. Immunol. 158:97). Typically, the rAAV vector genome will only retain the one or more ITR sequence so as to maximize the size of the transgene that can be efficiently packaged by the vector with the structural and non-structural protein coding sequences provided in trans (e.g., from a vector, such as a plasmid, or by stably integrating the sequences into a packaging cell). In embodiments of the invention the rAAV vector genome comprises at least one ITR sequence (e.g., AAV ITR sequence), optionally two ITRs (e.g., two AAV ITRs), which typically will be at the 5′ and 3′ ends of the vector genome and flank the heterologous nucleic acid, but need not be contiguous thereto. The ITRs can be the same or different from each other. Such a recombinant AAV (rAAV), is essentially a protein-based nanoparticle engineered to traverse the cell membrane, where it can ultimately traffic and deliver its DNA cargo into the nucleus of a cell. In the absence of Rep proteins, ITR-flanked transgenes encoded within rAAV can form circular concatemers that persist as episomes in the nucleus of transduced cells (Choi et al, J Virol. 2006; 80(21):10346-10356).

The term “inverted terminal repeat” or “ITR” includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see, e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, persistence, and/or provirus rescue, and the like.

The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

Further, the viral capsid or genomic elements can contain other modifications, including insertions, deletions and/or substitutions to thereby generate a derivative of the original.

The term “template” or “substrate” is used herein to refer to a polynucleotide sequence that may be replicated to produce the parvovirus viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wild-type or synthetic. A wild-type large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

As used herein, the term “associated promoter” when used to refer to an rAAV capsid protein as it is associated with a promoter, means the rAAV capsid protein is within the capsid of the rAAV viral particle, and the promoter is contained within the rAAV genome that is encapsidated within the particle. The promoter is typically operably linked to a transgene. Put another way, the rAAV capsid protein and the promoter are “in the context of a recombinant AAV particle”, referring to a rAAV genome where the promoter is operably linked to a transgene (e.g., as part of an expression cassette encoding a nucleic acid or protein of interest) and the rAAV genome is encapsidated by the rAAV particle.

As used herein, the term “amino acid” encompasses any naturally occurring amino acids, modified forms thereof, and synthetic amino acids, including non-naturally occurring amino acids.

Naturally occurring, levorotatory (L-) amino acids are shown in Table 2.

TABLE 2

Abbreviation

| Amino Acid | Three-Letter | One-Letter |
|---|---|---|
| Residue | Code | Code |
| Alanine | Ala | A |
| Arginine | Arg | R |
| Asparagine | Asn | N |
| Aspartic acid | Asp | D |
| (Aspartate) | | |
| Cysteine | Cys | C |
| Glutamine | Gln | Q |
| Glutamic acid | Glu | E |
| (Glutamate) | | |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described by Wang et al., (2006) Annu. Rev. Biophys. Biomol. Struct. 35:225-49. These unnatural amino acids can advantageously be used to chemically link molecules of interest to the AAV capsid protein.

TABLE 3
Amino Acid Residue Derivatives

| Modified Amino Acid Residue | Abbreviation |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

A “functional fragment” of a polypeptide or protein, as used herein, means a portion of a larger polypeptide that substantially retains its ability to enhance or increase transduction efficiency. For example, an isolated FerA domain polypeptide is a functional fragment of the larger ferlin protein.

The term “chimeric” refers to a molecule having two or more portions that are not naturally found together in the same molecule.

As used herein, the term “derivative” is used to refer to a polypeptide or genomic element which differs from a naturally occurring protein or genomic element by minor modifications (e.g., conservative amino acid substitutions, insertions, or deletions) to the naturally occurring polypeptide, but which substantially retains one or more biological activities of the naturally occurring protein. Minor modifications include, without limitation, changes in one or a few amino acid side chains, changes to one or a few amino acids (including deletions, insertions, and/or substitutions) (e.g., less than about 20, 15, 10, 9, 8, 7, 6, 5, 4, 3, or 2 changes), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol. As the term is used herein, the determination of a “derivative” is made prior to the amino acid sequence modification at the VP1/VP2 boundary discussed herein. However, the invention encompasses modified capsid proteins that are generated from capsid protein starting materials that are derivatives of an earlier (e.g., wt) capsid protein, or are further derivatized following the herein described amino acid sequence modifications to the VP1/VP2 boundary.

As used herein, the term “functional fragment” is used to refer to fragment of a polypeptide or genomic element which differs from a naturally occurring protein or genomic element by substantial deletion (e.g., greater than 20 a.a., or greater than 20 codon deletion), but which substantially retains one or more biological activities of the naturally occurring protein or nucleic acid. A “functional fragment” is also considered a portion of the longer (e.g., full length parent protein or genomic element). As the term is used herein, the determination of a “functional fragment” is made prior to the amino acid sequence modification at the VP1/VP2 boundary discussed herein. However, the invention encompasses modified capsid proteins that are generated from capsid protein starting materials that are functional fragments of an earlier (e.g., wt) capsid protein, or are further deleted (e.g., in other regions) following the herein described amino acid sequence modifications to the VP1/VP2 boundary.

The term “substantially retains,” as used herein, refers to a derivative, or other variant of a polypeptide or genomic element that retains at least one activity of the naturally occurring polypeptide or genomic element, about 50% of the activity of the naturally occurring polypeptide or genomic element, e.g., about 60%, 70%, 80%, 90%, 95%, 99% or more.

The term “conservative substitution” or “conservative substitution mutation” as used herein refers to a mutation where an amino acid is substituted for another amino acid that has similar properties, such that one skilled in the art of peptide chemistry would expect the secondary structure, chemical properties, and/or hydropathic nature of the polypeptide to be substantially unchanged. The following groups of amino acids have been historically substituted for one another as conservative changes: (1) ala, pro, gly, glu, asp, gln, asn, ser, thr; (2) cys, ser, try, thr; (3) val, ile, leu, met, ala, phe; (4) lys, arg, his; and (5) phe, tyr, trp, his. Other commonly accepted conservative substitutions are listed below.

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Where a derivative polypeptide or genome is or encodes a capsid proteins, i.e., VP1, VP2 and/or VP3, the derivative may be a chimeric, shuffled or capsid-modified derivative of one or more naturally occurring AAV viruses. In particular, the invention encompasses the provision of capsid protein sequences from different serotypes, clades, clones, or isolates of AAV within the same vector, i.e., pseudotyping.

Derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an otherwise identical AAV viral vector comprising a naturally occurring AAV capsid or genome. Increased efficiency of gene delivery may be effected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Viral capsids, vectors, and particles that have capsid proteins (VP1, and VP3, optionally VP2), where one or more of the VP1, VP2, and VP3 are only from a serotype(s) that differ from that of one or both of the other VP protein(s) are also encompassed in the invention (e.g., see WO 2019/216932, Rational Polyploid Adeno-Associated Virus Vectors And Methods Of Making And Using The Same, the contents of which are incorporated herein by reference).

Chimeric capsid proteins include those generated by recombination between two or more capsid coding sequences of naturally occurring AAV serotypes.

The term “corresponds to” or “corresponding to”, when used in reference to amino acid or nucleic acid sequence, is meant to indicate the amino acid or nucleic acid of a conserved protein or genetic sequence in several homologous molecules (e.g., those of various AAV serotypes/isolates). Such amino acid or nucleic acids that correspond to those present in a given protein or genetic sequence are readily known or determined by the skilled practitioner by referring to known/published sequence alignments or performing sequence identity/homology analysis as described herein. For convenience, the amino acid sequence alignment for capsid proteins of various AAV serotypes is provided in FIGS. 20A-20E.

### EMBODIMENTS

Aspects of the invention relate to the discovery of the ability of the AAV capsid in a viral particle to influence the activity of a promoter on an operably linked transgene, when the promoter-transgene is contained within that viral particle after infection of a target cell. For example, the respective promoter activity of two promoters (CBA and CBh) showed each promoter exhibits roughly the same cell type expression pattern when in the context of rAAV2 (both resulting in strong preferential expression in neurons when injected into the striatum, with minimal expression seen in oligodendrocytes). However, when the capsid is changed, the promoters exhibit different cell type expression patterns. In an AAV9 capsid, CBA results in strong preferential expression in neuronal cells, minimal expression in oligodendrocytes, and CBh results in significant expression in both neuronal cells and glial cells (oligodendrocytes). In an AAV2 capsid, differential cell type expression patterns were observed when comparing the hybrid chicken beta actin and human synapsin promoters. This shows that AAV capsids can exert an influence on promoter activity in different cell types.

We have also shown that this influence can be augmented by manipulation of the capsid proteins within a region referred to herein as the VP1/VP2 boundary. In AAV9, insertion of six glutamates (negatively charged) after amino acid 138 (VP1 numbering) disrupts a positively charged/basic region and shifts expression by the CBA promoter away from neurons to glial cells, to result in strong preferential expression in oligodendrocytes. Insertion of six alanine residues (neutral charge) at the same location in AAV9 VP1/2 had only a modest effect on the cell type specific activity of the CBA promoter, increasing preferential expression in neuronal cells only modestly as compared to unmodified VP1/2. Similarly, insertion of the six alanine residues at the same location in AAV2 VP1/2 did not alter the cell type expression of the CBA promoter as compared to unmodified VP1/2. The same insertion of six alanine residues in AAV9 VP1/2 had a more pronounced effect on the CBh promoter, resulting in strong preferential expression in neuronal cells. This was in contrast to the CBh promoter in unmodified AAV9 which resulted in expression in both neuronal and glial cells. These results indicate the existence of capsid-promoter interactions in vivo that confer preferential cell type expression by the promoter, and further indicate that such interactions can be altered (e.g., by modification of the capsid amino acid sequence) to influence cell type specific expression, thus altering the “permissiveness” of a promoter in a cell type.

Similar findings were observed with a different promoter (JeTI) in AAV9. The insertion of six alanine residues (neutral charge) in the AAV9 VP1/VP2 lead to the promotion of preferential neural expression by JeTI, reversing the preferential glial expression of JeTI seen with unaltered AAV9 VP1/VP2, indicating that the interaction of the capsid with the promoter to affect promoter permissiveness in cell types occurs with multiple promoters. In these cases, the effect of a modification is compared to the unmodified capsid proteins.

Capsid influence on promoter permissiveness was also observed using AAV2 and AAV8, indicating that the interaction of the capsid with promoters to affect promoter permissiveness in cell types occurs across different AAV serotypes. This phenomenon of capsid influence on promoter permissiveness occurs with different promoters and across different AAV serotypes. This phenomenon effects promoter permissiveness across a variety of different cell types.

### Modified AAV Capsid Proteins

One aspect of the invention relates to an AAV capsid protein that contains an amino acid sequence modification within an unstructured region of the VP1 protein. This amino acid region flanks the junction at which the unique sequences of VP1 end and the N-terminal sequences of VP2 begin, and is referred to herein as the VP1/VP2 boundary. This capsid protein, when present in a rAAV vector, alters gene expression of a promoter contained therein, increasing gene expression in certain cell types, and/or decreasing gene expression in other cell types. In this way, the “permissiveness” of the promoter in a specified cell type is altered. The findings disclosed herein indicate that selective modification of the amino acid sequence of the capsid protein(s) within this region can be made to affect transgene expression from an rAAV vector particle within specific cell types.

In one aspect, the AAV capsid protein has an amino acid sequence modification in one or more amino acids within the VP1/VP2 boundary that alters the permissiveness of an associated promoter when the promoter is within a target cell. The modification may be made to a wild type AAV capsid protein or to a derivative thereof (e.g., a capsid protein with one or more amino acid substitutions, or a previously generated chimeric capsid protein, etc.). The skilled practitioner will recognize that both VP1 and VP2 each contain at least a portion of the AAV VP1/VP2 boundary. In this way, the invention encompasses both VP1 and VP2 capsid proteins. The capsid protein of the invention may be from any AAV serotype or a derivative thereof.

The AAV capsid protein and the promoter are associated with one another in that they are in the context of a recombinant AAV (rAAV) particle. The rAAV particle will typically contain a transgene that is regulated by the promoter. Transgene expression from the rAAV particle in a host cell will be altered in one or more cells types upon infection, with respect to an appropriate control (e.g., an otherwise identical AAV particle lacking the modified capsid protein).

The location of the amino acid sequence modification is within a region of the VP1 protein that contains or is near the amino acids that are also contained in the N-terminus of VP2, referred to herein as the VP1/VP2 boundary. In some embodiments, the VP1/VP2 “boundary” (or “region of VP1/2 intersection”) is a unique region of the capsid sequence that is unstructured and thought to be inaccessible due to being internalized within the viral capsid structure. In some embodiments, the most C-terminal region of VP1 that is unique (not present in VP2) and the region of overlap with the N-terminal region of VP2 encompasses the “VP1/VP2 boundary”. In AAV9, this region is positively charged and basic. One or more of these characteristics are somewhat conserved across the various AAV serotypes and isolates. Without being bound by theory, it is thought that this region of the capsid interacts, either directly or indirectly, with the transgene regulatory sequences to influence promoter permissiveness discussed herein.

The modification may be to the nucleic acids encoding the one or more amino acids. As described herein, one can make any alteration to such nucleic acids to obtain the amino acids modifications discussed herein.

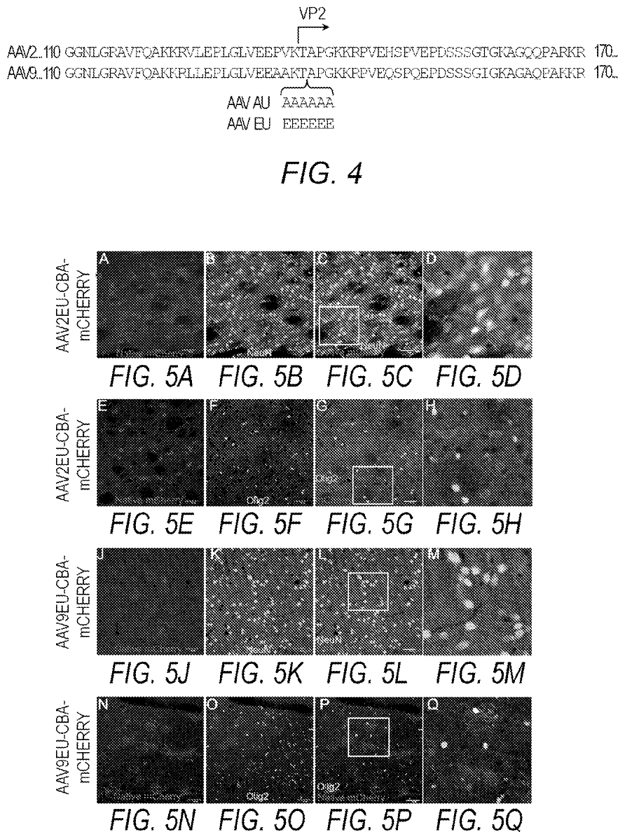

In some embodiments, the boundary comprises amino acids that are N-terminal and C-terminal to the two amino acids that are referred to herein as the VP1/VP2 junction. The VP1/VP2 junction is formed by the amino acid of VP1 that is directly (N-terminal) adjacent to the first amino acid of VP2 (shown by the arrow in FIG. 4). These two amino acids together form the junction. The first amino acid of VP2 corresponds to T 138 of VP1 in AAV9, and is highly conserved in all AAV serotypes as shown in FIGS. 20A-20E. For AAV 4 and AAV5, the VP1/VP2 junction is between amino acids 136 and 137. For AAV2, AAV3, AAV6, AAV7, AAV8, AAV9 and AAV10, the VP1/VP2 junction is between amino acids 137 and 138. The VP1/VP2 boundary, or junction region, includes a number of amino acids on either side of the junction, e.g., about 5, 10, 15, 20, 25, or 30 amino acids on either side of the junction. In some embodiments, the boundary is amino acids 110-170 of VP1 in AAV9 (SEQ ID NO:14) or the corresponding amino acids in other AAV serotypes (e.g., see FIGS. 20A-20E showing the alignment of capsid proteins in various AAV serotypes). This embodiment of the boundary is shown for AAV2 and AAV9, respectively, in FIG. 4. In some embodiments, the boundary corresponds to amino acids 115-165, 120-160, 125-155, 130-150, or 135-140 of AAV9 capsid (e.g., the corresponding amino acids in other AAV serotypes capsid protein or derivative thereof shown in Table 1). In some embodiments, the VP1/VP2 boundary corresponds to amino acids 120-150 of AAV9 capsid, or 125-145 of AAV9 capsid, or 130-140 of AAV9 capsid (e.g., in other AAV serotypes capsid protein or derivative thereof shown in Table 1). The designation of all amino acid positions in the capsid proteins described herein are made in reference to VP1 numbering, although the skilled practitioner will recognize that the parts of this region that fall C-terminal to the junction are also in VP2, and this can also be represented by the corresponding VP2 numbering.

In some embodiments, the particle can be a rational haploid, i.e., where at least one of the viral capsid proteins (VP1, VP3, optionally VP2) present within the capsid is only from a completely different serotype than one or more of the other capsid proteins.

The amino acid sequence modification is at one or more amino acids within the VP1/VP2 boundary. In one embodiment, the modification is at one or more amino acids from a.a. 110-170, 115-165, 120-160, 125-155, 130-150, 135-140, or from a.a. 110-139, 115-139, 120-139, 125-139, 130-139, 135-139, or from a.a. 110-138, 115-138, 120-138, 125-138, 130-138, 135-138, or from a.a. 138-150, 138-155, 138-160, 138-165, 138-170, 139-150, 139-155, 139-160, 139-165, 139-170 of VP1 in AAV9 (SEQ ID NO:14) or the corresponding amino acids in another AAV serotype (e.g., AAV1, 2, 3, 4, 5, 6, 7, 8, 10, 11, 12, 13, or any other AAV serotype or isolate shown in Table 1). In some embodiments of the invention, the amino acid sequence modification is at amino acid 138 or 139 of VP1 in AAV9 or the corresponding amino acids in another AAV serotype or isolate. In some embodiments of the invention, the amino acid sequence modification is an insertion after (C-terminal to) to amino acid 138 or 139 of VP1 in AAV9 or the corresponding amino acids in another AAV serotype.

In some embodiments, the amino acid sequence modification alters the electrostatic charge of the VP1/VP2 boundary of the capsid that forms the particle, to thereby alter the permissiveness of the promoter within the cell.

In some embodiments, the amino acid sequence modification comprises a mutation selected from the group consisting of an insertion, a deletion, a substitution, and combinations thereof. The modification may be anywhere in the VP1/VP2 boundary as discussed directly above. In some embodiments, the modification is at the VP1/VP2 junction, e.g., an insertion at the junction. In some embodiments, the modification is within 1, 2, 3, 4, or 5 amino acids of the VP1/VP2 junction, either N-terminal or C-terminal to the VP1/VP2 junction.

In some embodiments, the modification comprises, consists essentially of, or consists of a substitution of 1, 2 or fewer, 3 or fewer, 4 or fewer, 5 or fewer, 6 or fewer, 7 or fewer, 8 or fewer, 9 or fewer, 10 or fewer, 12 or fewer, 15 or fewer, 20 or fewer, 25 or fewer, 30 or fewer, 40 or fewer, or 50 or fewer of the amino acids within the capsid protein by another amino acid (naturally occurring, modified and/or synthetic), and/or a deletion of 1, 2 or fewer, 3 or fewer, 4 or fewer, 5 or fewer, 6 or fewer, 7 or fewer, 8 or fewer, 9 or fewer, 10 or fewer, 12 or fewer, 15 or fewer, 20 or fewer, 25 or fewer, 30 or fewer, 40 or fewer, or 50 or fewer amino acids, and/or an insertion of 1, 2 or fewer, 3 or fewer, 4 or fewer, 5 or fewer, 6 or fewer, 7 or fewer, 8 or fewer, 9 or fewer, 10 or fewer, 12 or fewer, 15 or fewer, 20 or fewer, 25 or fewer, 30 or fewer, 40 or fewer, or 50 or fewer amino acids, or any combination of substitutions, deletions and/or insertions. It is thought that insertions of up to 200 amino acids can be tolerated when made at or near the VP1/VP2 junction. In one embodiment, the amino acid sequence modification is an insertion of between 1 and 200 amino acids (e.g., 2, 3, 3 or more, 4, 4 or more, 5, 5 or more, 6, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more), and is made C-terminal to the amino acid corresponding to amino acid 138, 139 or 140 of AAV9 VP1 or the corresponding amino acids in another AAV serotype (e.g., in any AAV serotype capsid protein or derivative thereof shown in Table 1).

In some embodiments, the substitution(s) is conservative. In some embodiments, the substitution(s) introduces a positively charged amino acid(s) (e.g., in place of a neutral or negatively charged amino acid(s)). In some embodiments, the substitution(s) introduces a neutral charged amino acid(s) (e.g., in place of a positively or negatively charged amino acid(s)). In some embodiments, the substitution(s) introduces a negatively charged amino acid(s) (e.g., in place of a neutral or positively charged amino acid(s)).

In certain embodiments, the amino acid sequence modification is an insertion of one or more amino acid residues, e.g., 1-3, 3 or more, 4 or more, 5 or more, 6 or more, 7 or more, 8 or more, 9 or more, 10 or more. Insertions of up to 200 amino acids will be tolerated at the site and are thought to affect the promoter modification activity of the capsid proteins observed herein. In some embodiments, the insertion is from 1-20 amino acid residues, e.g., from 4-16 amino acid residues, from 6-14 amino acid residues, or up to 200 amino acid residues. In some embodiments, the amino acid sequence modification is an insertion of 3 or more amino acid residues, e.g., 3-6 amino acid residues, 3-8 amino acid residues, 3-10 amino acid residues, 3-12 amino acid residues, 3-15 amino acid residues, or 3-20 amino acid residues.

In certain embodiments, the insertion is C-terminal to amino acid 138, or C-terminal to amino acid 139 of AAV9 or the corresponding amino acid position in another AAV serotype (e.g., AAV1, 2, 3, 4, 5, 6, 7, 8, 10, 11, 12, 13, or any other AAV serotype or isolate shown in Table 1).

In some embodiments, the amino acid insertion (e.g., C-terminal to amino acid corresponding to 138 or 139 of AAV9) is represented as (X₃-X₂₀₀), where X is any negative charge amino acid or combination thereof, or combination of amino acids that have together result in an overall negative charge.

In some embodiments, the amino acid insertion (e.g., C-terminal to amino acid corresponding to 138 or 139 of AAV9) is represented as (X3-X200), where X is any neutral charge amino acid or combination thereof, or combination of amino acids that have together result in an overall neutral charge.

In some embodiments, the amino acid insertion (e.g., C-terminal to amino acid corresponding to 138 or 139 of AAV9) is represented as (X3-X200), where X is any positive charge amino acid or combination thereof, or combination of amino acids that have together result in an overall positive charge.

In certain embodiments, the amino acid sequence modification preserves nuclear localization signals (putative or demonstrated) and phospholipase domains present in the capsid protein. Putative nuclear localization signals are known in the art (e.g., Johnson et al., J. Virol. 84:8888 (2010)). Basic regions within the capsid proteins identified as putative nuclear localization signals in AAV2 that are found within the VP1/VP2 boundary include, without limitation, QAKKR (known as BR1) found in VP1 at amino acid positions corresponding to 120-124 of AAV2 VP1 (SEQ ID NO:6), PGKKR (known as BR2), found just C-terminal to the VP2 translation start site at amino acid positions corresponding to 140-144 of AAV2 VP1 (SEQ ID NO:6), and PARKR (known as BR3) at amino acid positions corresponding to 168-172 of AAV2 VP1 (SEQ ID NO:6). A phospholipase domain HA (known as PLA2) is found in the amino acid sequence unique to VP1.

The amino acid sequence modification may be, for example, an insertion between amino acids corresponding to 137 and 138 of AAV9, or between amino acids corresponding to 138 and 139 of AAV9 (e.g., in any AAV serotype capsid protein or derivative thereof shown in Table 1).

In some embodiments, the amino acid sequence modification results in a modification to the amino acid sequence of only VP1, such as a modification to one or more amino acids that are N-terminal to the VP2 translation start site. In some embodiments, the amino acid sequence modification results in a modification of the amino acids sequence of both VP1 and VP2, such as a modification to one or more amino acids that are C-terminal to the VP2 translation start site. When a modification comprises more than one insertion, substitution or deletion, the combination may be result in a modification to only VP1, only VP2, or to both VP1 and VP2.

The modified capsid protein alters promoter permissiveness based on cell type. Promoter permissiveness, as the term is used herein, refers to whether or not a promoter operates in a given cell type. An alteration of promoter permissiveness can be an increase or a decrease in promoter function in the cell type, as indicated by an increase or decrease in operably linked transgene expression.

The altered permissiveness that leads to a decrease in promoter function may be detected by a decrease in transgene expression in a cell type relative to the level of expression from an otherwise identical rAAV particle comprising unmodified capsid protein, e.g., a relative decrease of 10%, 20%, 30%, 40%, 50%, 60%, 70%, 80%, 90%, or more.

The altered permissiveness that leads to an increase in promoter function may be detected by an increase in transgene expression in a cell type relative to the level of expression from an otherwise identical rAAV particle comprising unmodified capsid protein, e.g., a relative increase of 10%, 20%, 30%, 40%, 50%, 60%, 70%, 80%, 90%, 100%, 150%, 250%, 500%, or more.

The altered permissiveness may be a decrease in promoter function in one cell type combined with an increase in promoter function in another cell type, detected by a decrease in transgene expression in one cell type combined with an increase in transgene expression in a different cell type.

In certain embodiments, the amino acid sequence modification to the capsid protein increases expression of a transgene from the associated promoter in a first cell type and/or decreases expression of a transgene from the associated promoter in a second cell type. In some embodiments, expression is increased in the first cell type by 10%, 20%, 30%, 40%, 50%, 60%, 70%, 80%, 90%, 100%, 150%, 250%, 500%, or more and/or expression is decreased in the second cell type by 10%, 20%, 30%, 40%, 50%, 60%, 70%, 80%, 90%, or more relative to the level of expression from a rAAV particle comprising unmodified capsid protein. Expression levels can be quantitated using methods known in the art and as described herein, such as measuring the level or activity of a reporter protein expressed from a reporter gene operably linked to the promoter, e.g., using an enzymatic assay or immunoassay.

Promoter permissiveness can be determined by identifying preferential expression conferred by the promoter in the rAAV particle in a given cell type or types. As the term is used herein, “preferential expression” refers to increased expression of a promoter in one cell type over another cell type. In one embodiment, preferential expression is greater than 50%, 60%, 65%, 70%, 75%, 80%, 85%, 90%, 95% expression in one cell type relative to that in another cell type. Relative expression of a promoter in the respective cell types can be determined using an operably linked reporter transgene and detecting co-localization of the reporter with markers specific for the cell type of interest. In some embodiments, the cell type of interest is a neuronal cell and co-localization of the reporter is with one or more neuronal markers (e.g., NeuN)). In some embodiments the cell type of interest is a glial cell (e.g., an oligodendrocyte) and co-localization of the reporter is with a glial cell marker (e.g., Oligo 2).

The cell in which permissiveness is altered may be any cell type including, but not limited to, neural cells (including cells of the peripheral and central nervous systems, in particular, brain cells such as neurons and glial cells (e.g., oligodendrocytes)), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin. Furthermore, the cells may be dividing or non-dividing.

In some embodiments, the cell is a CNS cell. In some embodiments, the cell in which permissiveness is increased or decreased is a neuronal cell or a glial cell. In some embodiments, the neuronal cell is a medium spiny neuron, a cholinergic interneuron, or a GABAergic interneuron. In some embodiments, the glial cell is an oligodendrocyte, microglia, or astrocyte.

In certain embodiments, the amino acid sequence modification to the capsid protein increases expression of a transgene from the associated promoter in oligodendrocytes and/or decreases expression of a transgene from the associated promoter in neurons. In some embodiments, expression is increased in oligodendrocytes by 10%, 20%, 30%, 40%, 50%, 60%, 70%, 80%, 90%, 100%, 150%, 250%, 500%, or more and/or expression is decreased in neurons by 10%, 20%, 30%, 40%, 50%, 60%, 70%, 80%, 90%, or more relative to the level of expression from a rAAV particle comprising unmodified capsid protein. Expression levels can be quantitated using methods known in the art and as described herein, such as measuring the level or activity of a reporter protein expressed from a reporter gene operably linked to the promoter, e.g., using an enzymatic assay or immunoassay.

In some embodiments, the amino acid sequence modification is an insertion of one or more residues having an overall negative charge. The overall charge of an insertion is calculated by adding the net charge of each individual amino acid, with basic amino acids (histidine, lysine, and arginine) providing a +1 charge, acidic amino acids (glutamate and aspartate) providing a −1 charge, and the remaining amino acids being neutral. An overall negative charge is charge of −1 or lower. In some embodiments, the amino acid sequence modification is an insertion of 2 or more negatively charged amino acid residues, e.g., glutamate and/or aspartate residues, e.g., 2-4, 2-6, 2-8, 2-10, or 2-12 residues. In some embodiments, the amino acid sequence modification is an insertion of 2 or more glutamate residues, e.g., 2-4, 2-6, 2-8, 2-10, or 2-12 glutamate residues. In some embodiments, the amino acid sequence modification is an insertion of 6 glutamate residues.

In some embodiments, the amino acid sequence modification is an insertion of one or more residues having an overall positive charge. An overall positive charge of an insertion is charge of +1 or higher. In some embodiments, the amino acid sequence modification is an insertion of 2 or more positively charged amino acid residues, e.g., histidine, lysine, and/or arginine residues, e.g., 2-4, 2-6, 2-8, 2-10, or 2-12 residues. In some embodiments, the amino acid sequence modification is an insertion of substance P peptide (RPKPQQFFGLM (SEQ ID NO:19)).

In certain embodiments, the amino acid sequence modification increases expression of a transgene in neurons and/or decreases expression of a transgene in oligodendrocytes. In some embodiments, expression is increased in neurons by 10%, 20%, 30%, 40%, 50%, 60%, 70%, 80%, 90%, 100%, 150%, 250%, 500%, or more and/or expression is decreased in oligodendrocytes by 10%, 20%, 30%, 40%, 50%, 60%, 70%, 80%, 90%, or more relative to the level of expression from a rAAV particle comprising unmodified capsid protein.

In some embodiments, the amino acid sequence modification is an insertion of residues having an overall neutral charge. An overall neutral charge of an insertion has a net charge of 0 as determined as described above. In some embodiments, the amino acid sequence modification is an insertion of 2 or more neutral amino acid residues, e.g., 2-4, 2-6, 2-8, 2-10, or 2-12 neutral amino acid residues. In some embodiments, the amino acid sequence modification is an insertion of 2 or more alanine residues, e.g., 2-4, 2-6, 2-8, 2-10, or 2-12 alanine residues. In some embodiments, the amino acid sequence modification is an insertion of 6 alanine residues.

The capsid protein or derivative thereof that is modified may be from any AAV serotype, e.g., any of the serotypes listed in Table 1. In some embodiments, the capsid protein is from a serotype selected from the group consisting of AAV1, AAV2, AAV3, AAV4, AAV5, AAV6, AAV7, AAV8, AAV9, AAV10, AAV11, AAV12, and AAV13. The capsid protein may be a non-naturally occurring capsid protein, e.g., a chimeric capsid protein comprising sequences from two or more different AAV serotypes. In some embodiments, the chimeric capsid protein comprises the VP1/VP2 boundary of AAV9. The specific genomic coding sequences and amino acid sequences for the capsid proteins of the different AAV serotypes are found under the GenBank accession numbers provided in Table 1. Accession numbers for the capsid sequence of the most common AAV serotypes are provided shown in Table 4. The amino acid sequences of capsids for AAV1-AAV10, and an alignment showing of those sequences is shown in FIGS. 20A-20E.

TABLE 4
Exemplary AAV Genome and Capsid Accession Nos.

| Virus and | Genome | Capsid/VP1 |
|---|---|---|
| Serotype | Accession No. | Accession No. |
| AAV1 | NC_002077.1 | NP_049542.1 |
| AAV2 | NC_001401.2 | YP_680426.1 |
| AAV3A | NC_001729.1 | NP_043941.1 |
| AAV3B | NC_001863.1 | NP_045760.1 |
| AAV4 | NC_001829.1 | NP_044927.1 |
| AAV5 | NC_006152.1 | YP_068409.1 |
| AAV6 | NC_001862.1 | NP_045758.1 |
| AAV7 | AF513851.1 | AAN03855.1 |
| AAV8 | AF513852.1 | AAN03857.1 |
| AAV9 | AY530579.1 | AAS99264.1 |
| AAV10 | AY631965.1* | AAT46337.1 |
| AAV11 | AY631966.1* | AAT46339.1 |
| AAV12 | ABI16639.1 | DQ813647 |
| AAV13 | EU285562.1 | ABZ10812.1 |

*Incomplete sequence

The AAV particle comprising the modified capsid protein described herein may be any AAV serotype, e.g., any of the serotypes listed in Table 1. In some embodiments, the rAAV particle is selected from the group consisting of AAV1, AAV2, AAV3, AAV4, AAV5, AAV6, AAV7, AAV8, AAV9, AAV10, AAV11 and AAV13.

It will be apparent to those skilled in the art that the amino acid sequences of the modified capsid protein of the invention can further be modified to incorporate other modifications as known in the art to impart desired properties. Such modifications are referred to herein as “derivatives”. As nonlimiting possibilities, the capsid protein can be modified to incorporate targeting sequences (e.g., RGD) or sequences that facilitate purification and/or detection. For example, the capsid protein can be fused to all or a portion of glutathione-S-transferase, maltose-binding protein, a heparin/heparan sulfate binding domain, poly-His, a ligand, and/or a reporter protein (e.g., Green Fluorescent Protein, β-glucuronidase, β-galactosidase, luciferase, etc.), an immunoglobulin Fc fragment, a single-chain antibody, hemagglutinin, c-myc, FLAG epitope, and the like to form a fusion protein. Methods of inserting targeting peptides into the AAV capsid are known in the art (see, e.g., international patent publication WO 00/28004; Nicklin et al., (2001) Mol. Ther. 474-181; White et al., (2004) Circulation 109:513-319; Muller et al., (2003) Nature Biotech. 21:1040-1046.

The promoter that is affected by the capsid protein (which has altered permissiveness when associated with the capsid protein in the context of an AAV particle in a cell, as described herein) may be any promoter that is suitable for use in a rAAV particle. In some embodiments, the promoter is a ubiquitous promoter. As used herein, a ubiquitous promoter is one that is functional in a wide range of tissue and cell types, although not necessarily every cell type. Examples of ubiquitous promoters include, without limitation, cytomegalovirus (CMV) immediate-early enhancer and chicken beta-actin (CAG), cytomegalovirus (CMV), CMV/chicken β-actin (CMV/β-actin), elongation factor 1α (EF1α), phosphoglycerate kinase, ubiquitin C (UbC), CBA, and CBh. In some embodiments, the promoter is a tissue specific promoter. The promoter may be a constitutive promoter or an inducible/regulatable promoter (see, e.g., WO 2011/126808 and WO 2013/04943).

In some embodiments, the promoter is the JeTI (Karumuthil-Melethil et al., Hum. Gene Ther. 27:509 (2016)), human synapsin promoter (hSYN1) (McClean et al., Neuroscience Letters, 576 (2014) p′73-′78), cytomegalovirus (CMV) promoter, or a CB7 promoter. Other possible promoters include, without limitation, the human β-actin promoter, the human elongation factor-1 α promoter, the simian virus 40 promoter, and the herpes simplex virus thymidine kinase promoter. See, e.g., Damdindorj et al., PLoS ONE 9(8):e106472 (2014). In some embodiments, the promoter is selected from the dihydrofolate reductase promoter, the phosphoglycerol kinase (PGK) promoter, the rhodopsin kinase promoter, the rhodopsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter can be a synthetic promoter. The promoter sequences thereof are known to one of skill in the art or available publicly, such as in the literature or in databases, e.g., GenBank, PubMed, or the like.

Exemplary liver-specific promoters that can be utilized include the ornithine transcarbamylase (OTC) promoter and the alpha 1-antitrypsin (AAT) promoter. Other liver-specific promoters include, but are not limited to, the albumin promoter, hepatitis B virus core promoter, thyroxin binding globulin (TGB) promoter and the LSP1 promoter (Cunningham et al. (2008) Molecular Therapy 16:1081-1088). Promoters active in skeletal muscle include, without limitation, those from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are also known various other tissues, e.g., for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15(1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)). Promoters for brain or other CNS expression include, without limitation, promoters: Synapsinl for all neurons, CaMKII alpha for excitatory neurons, GAD67 or GAD65 or VGAT for GABAergic neurons, etc. Promoters for lung expression can be used, for example SP-B promoter. Promoters for endothelial cells can be used, for example ICAM. Promoters for hematopoietic cells expression can be used, for example IFN beta or CD45 promoters. Promoters for osteoblasts can be used, for example OG-2. Tissue specific promoters for heart (e.g., NSE), and eye (e.g., MSK) may also be used.

In certain embodiments the promoter is a synthetic promoter (e.g., JeTI, SPc5-12, 2R5Sc5-12, dMCK, or tMCK). Other synthetic promoters include SP1 elements and the chicken beta actin promoter (CB or CBA). A synthetic promoter may comprise, for example, one or more regions of known promoters, regulatory elements, transcription factor binding sites, enhancer elements, repressor elements, and the like, and combinations thereof.

The promoter may be inducible (e.g., a promoter induced by the presence of an inducer, the absence of a repressor, or any other suitable physical or chemical condition that induces transcription from the inducible promoter. The terms “inducer”, “inducing conditions” and suchlike should be understood accordingly. By way of non-limiting example, an inducible promoter for use in embodiments of the invention may be a small molecule-inducible promoter, a tetracycline-regulatable (e.g., inducible or repressible) promoter, an alcohol-inducible promoter, a steroid-inducible promoter, a mifepristone (RU486)-inducible promoter, an ecdysone-inducible promoter, a rapamycin-inducible promoter, a metallothionein-inducible promoter, a hormone-inducible promoter, a cumate-inducible promoter, a temperature-inducible promoter, a pH-inducible promoter and a metal-inducible promoter.

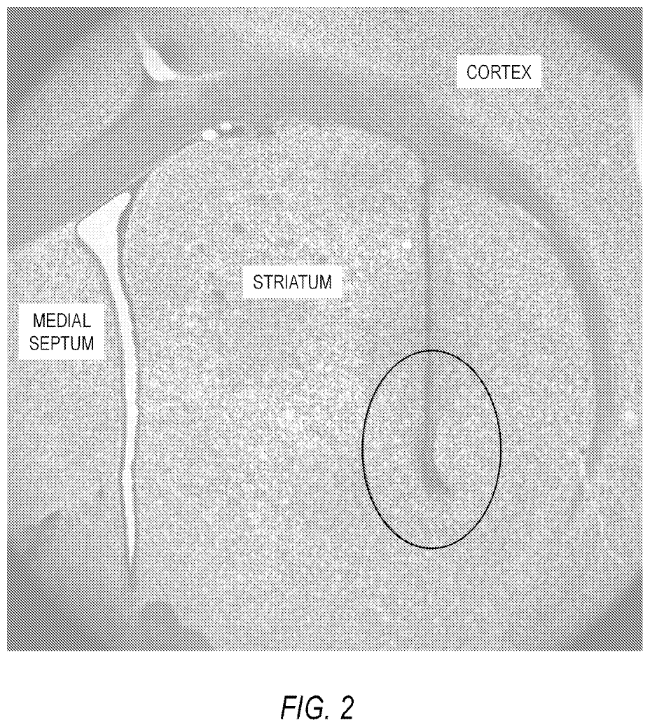

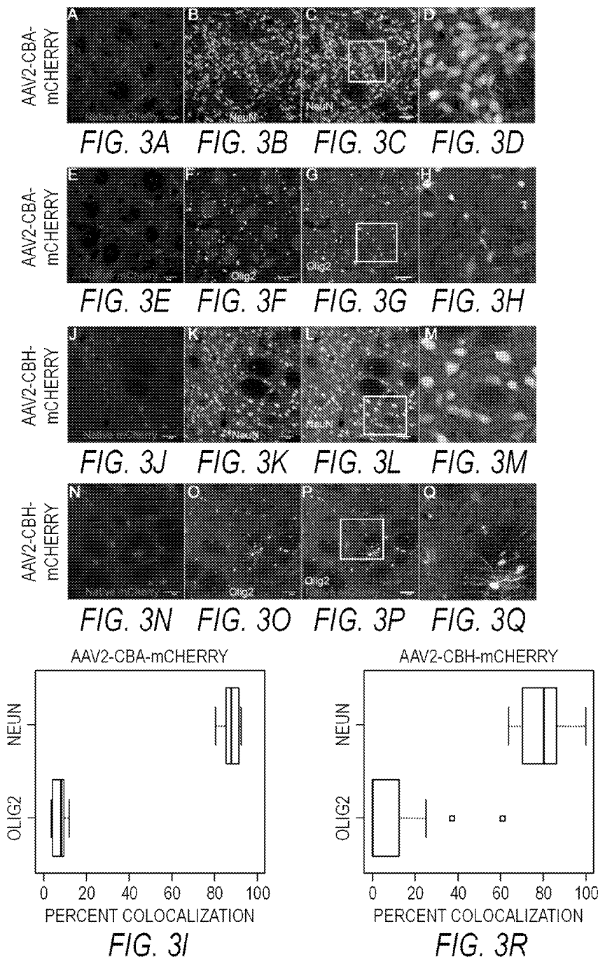

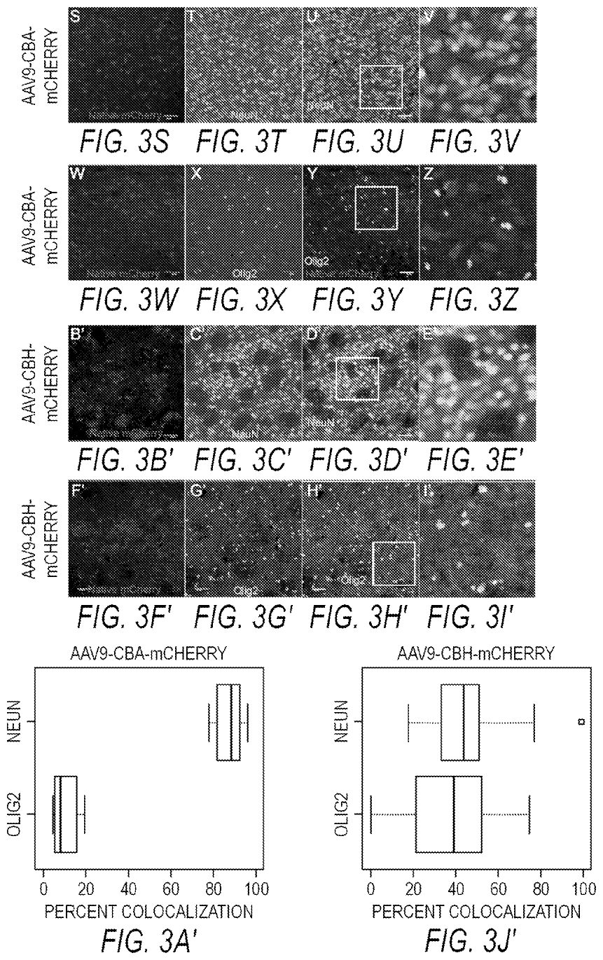

The inducible promoter may be induced by reduction of temperature, e.g., a cold-shock responsive promoter. In some embodiments, the inducible promoter is a synthetic cold-shock responsive promoter derived from the S1006a gene (calcyclin) of CHO cells. The temperature sensitivity of the S1006a gene (calcyclin) promoter was identified by Thaisuchat et al., 2011 (Thaisuchat, H. et al. (2011) ‘Identification of a novel temperature sensitive promoter in cho cells’, BMC Biotechnology, 11. doi: 10.1186/1472-6750-11-51), which is incorporated herein by reference. In some embodiments, the inducible promoter is one of the synthetic cold-shock responsive promoters shown in FIG. 2 of Thaisuchat et al., 2011. These promoters are induced by decrease of temperature as shown in FIG. 3 of Thaisuchat et al., 2011. Most of these synthetic promoter constructs show expression similar to the known promoter SV40 at 37° C. and are induced by 2-3 times when the temperature is reduced to 33° C. In some embodiments, the inducible promoter is sps5 from FIG. 2 of Thaisuchat et al., 2011. In some preferred embodiments, the inducible promoter is sps8 from FIG. 2 of Thaisuchat et al., 2011.

The inducible promoter may be a pH inducible promoter. As the term is used herein, a pH inducible promoter is induced by reduction or increase of pH to which cells comprising the promoter are exposed. Suitably, the inducible promoter may be induced by reduction of pH, i.e., a promoter inducible under acidic conditions. Suitable acid-inducible promoters are described in Hou et al., 2016 (Hou, J. et al. (2016) ‘Isolation and functional validation of salinity and osmotic stress inducible promoter from the maize type-II H+-pyrophosphatase gene by deletion analysis in transgenic tobacco plants’, PLoS ONE, 11(4), pp. 1-23. doi: 10.1371/journal.pone.0154041), which is incorporated herein by reference.

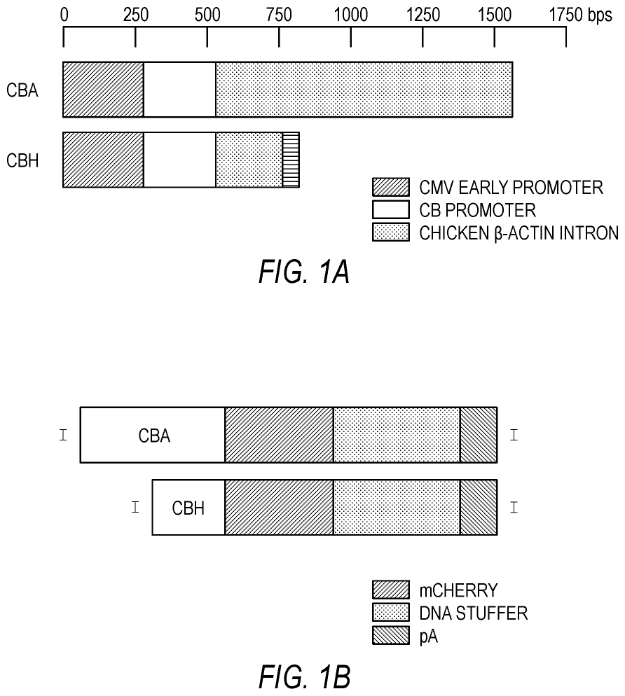

In some embodiments, the inducible promoter is a synthetic promoter inducible under acidic conditions derived from the YGP1 gene or the CCW14 gene. The inducibility by acidic conditions of the YGP1 gene or the CCW14 gene was studied and improved by modifying transcription factor binding sites by Rajkumar et al., 2016 (Rajkumar, A. S. et al. (2016) ‘Engineering of synthetic, stress-responsive yeast promoters’, 44(17). doi: 10.1093/nar/gkw553), which is incorporated herein by reference. In some embodiments, the inducible promoter is one of the synthetic promoters inducible under acidic conditions in FIGS. 1A, 2A, 3A and 4A of Rajkumar et al., 2016. These promoters are induced by decrease of pH as shown in FIGS. 1B, 2B, 3B and 4B of Rajkumar et al., 2016. Most of these synthetic promoters are induced by up to 10-15 times when the reduced from pH 6 to pH 3. In some preferred embodiments, the inducible promoter is YGP1pr from FIG. 1 of Rajkumar et al., 2016. In other preferred embodiments, the inducible promoter is YGP1pr from FIG. 1 of Rajkumar et al., 2016

The inducible promoter may be osmolarity-induced (referred to herein as osmolarity-inducible promoters). Suitable promoters induced by osmolarity are described in Zhang et al. (Molecular Biology Reports volume 39, pages 7347-7353(2012)) which is incorporated herein by reference.

The inducible promoter may be induced by addition of a specific carbon source, e.g., a non-sugar carbon source, referred to herein as carbon source-inducible promoters. Alternatively, the inducible promoter may be induced by withdrawal or the absence of a carbon source. Suitable promoters induced by the presence or absence of various carbon sources are described in Weinhandl et al., 2014 (Weinhandl, K. et al. (2014) ‘Carbon source dependent promoters in yeasts’, Microbial Cell Factories, 13(1), pp. 1-17. doi: 10.1186/1475-2859-13-5), which is incorporated herein by reference.

In addition, alcohol (e.g., ethanol)-inducible promoters may be used. Suitable promoters induced by ethanol are described in Matsuzawa et al. (Applied Microbiology and Biotechnology volume 97, pages 6835-6843(2013)), which is incorporated herein by reference. Also included are amino acid-inducible promoters. These are induced by addition of one or more amino acids. In some embodiment, the amino acid may be an aromatic amino acid. In some embodiments, the amino acid may be GABA (gamma aminobutyric acid), which is also a neurotransmitter. Examples of promoter induced by aromatic amino acids and GABA are described in Kim et al. (Applied Microbiology and Biotechnology, volume 99, pages 2705-2714(2015)) which is incorporated herein by reference.

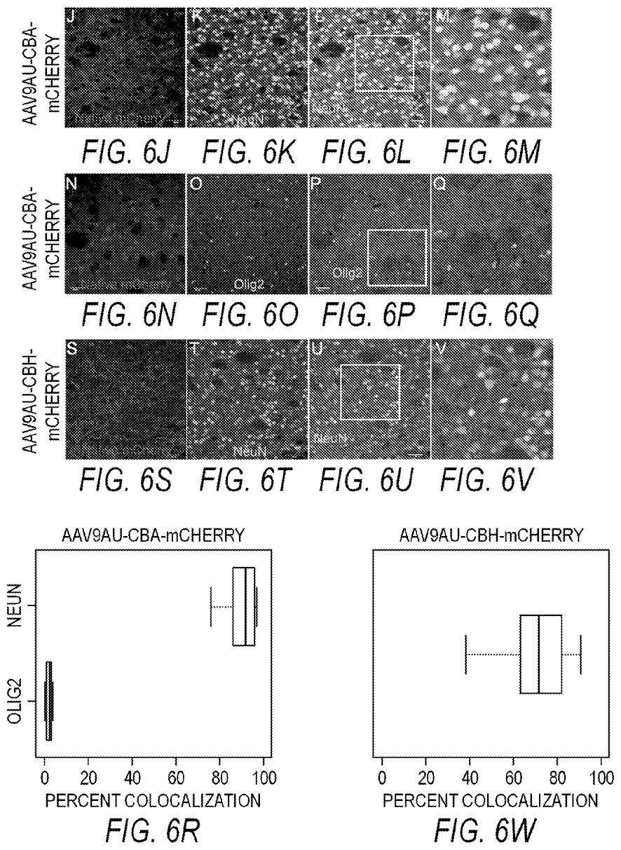

The inducible promoter may be induced by a steroid hormone (referred to herein as hormone inducible promoters). Suitably, the steroid hormone may be ecdysone. A mammalian ecdysone-inducible system was created by No, Yao and Evans (No, D., Yao, T. P. and Evans, R. M. (1996) ‘Ecdysone-inducible gene expression in mammalian cells and transgenic mice’, Proceedings of the National Academy of Sciences of the United States of America, 93(8), pp. 3346-3351. doi: 10.1073/pnas.93.8.3346), which is incorporated herein by reference. Expression of a modified ecdysone receptor in mammalian cells allows expression from an ecdysone responsive promoter to be induced upon addition of ecdysone as shown in FIG. 2 of No, Yao and Evans, 1996. This system showed lower basal activity and higher inducibility than the tetracycline-inducible system as shown in FIG. 6 of No, Yao and Evans, 1996. A suitable commercially available inducible system is available from Agilent technologies and is described in Agilent Technologies (2015) ‘Complete Control Inducible Mammalian Expression System Instruction Manual’, 217460, which is incorporated herein by reference.

In some embodiments, the promoter may be induced by the presence or absence of tetracycline or its derivatives (referred to herein as tetracycline-regulated promoters). In one embodiment, the promoter is induced in the absence of tetracycline or its derivatives is the promoter in the tet-OFF system. In the tet-OFF system, tetracycline-controlled transactivator (tTA) allows transcriptional activation of a tTA-dependent promoter in the absence of tetracycline or its derivatives. tTA and the tTA-dependent promoter were initially created by Gossen and Bujard, 1992 (Gossen, M. and Bujard, H. (1992) ‘Tight control of gene expression in mammalian cells by tetracycline-responsive promoters’, Proceedings of the National Academy of Sciences of the United States of America, 89(12), pp. 5547-5551. doi: 10.1073/pnas.89.12.5547), which is incorporated herein by reference. tTA was created by fusion of the tetracycline resistance operon (tet repressor) encoded in Tn10 of Escherichia coli with the activating cycline-controlled transactivator (tTA) and the tTA-dependent promoter was created by combining the tet operator sequence and a minimal promoter from the human cytomegalovirus promoter IE (hCMV-IE). When tetracycline or its derivatives are added, tTA can no longer bind its target sequence within the tTA-dependent promoter and there is no expression from the tTA-dependent promoter. This is shown in FIG. 1A and explained on page s96 of Jaisser, 2000 (Jaisser, F. (2000) ‘Inducible gene expression and gene modification in transgenic mice’, Journal of the American Society of Nephrology, 11(SUPPL. 16), pp. 95-100), which is incorporated herein by reference. The mechanism of the conformational change brought by binding of tetracycline or its derivatives to tTA is described in Orth et al., 2000 (Orth, P. et al. (2000) ‘Structural basis of gene regulation by the tetracycline inducible Tet repressor-operator system’, Nature Structural Biology, 7(3), pp. 215-219. doi: 10.1038/73324), which is incorporated herein by reference. Binding of tetracycline to TetR increases the separation of the attached DNA binding domains which abolishes the affinity of TetR for its operator DNA.

In one embodiment, the promoter is induced by presence of tetracycline or its derivatives, such as the promoter that is the tet-ON system. In the tet-ON system, a reverse tetracycline-controlled transactivator (rtTA) allows transcriptional activation of a tTA-dependent promoter in the presence of tetracycline or its derivatives as described in Gossen et al. (Science 23 Jun. 1995: Vol. 268, Issue 5218, pp. 1766-1769 DOI: 10.1126/science.7792603), which is incorporated herein by reference. In the absence of tetracycline or its derivatives, tTA can no longer bind its target sequence within the tTA-dependent promoter and there is no expression from the tTA-dependent promoter. This is shown in FIG. 1B and explained on page s96 of Jaisser, 2000 (Jaisser, F. (2000) ‘Inducible gene expression and gene modification in transgenic mice’, Journal of the American Society of Nephrology, 11(SUPPL. 16), pp. 95-100), which is incorporated herein by reference.

In some embodiments an improved variant of the reverse tetracycline-controlled transactivator (rtTA) is used. Examples of improved variants are described in Table 1 of Urlinger et al., 2000 (Urlinger, S. et al. (2000) ‘Exploring the sequence space for tetracycline-dependent transcriptional activators: Novel mutations yield expanded range and sensitivity’, Proceedings of the National Academy of Sciences of the United States of America, 97(14), pp. 7963-7968. doi: 10.1073/pnas.130192197), which is incorporated herein by reference. Variants rtTA-S2 and rtTA-M2 were shown to have lower basal activity in FIG. 3 of Urlinger et al., 2000, which indicates minimal background expression from the tTA-dependent promoter in the absence of tetracycline or its derivatives. Additionally, rtTA-M2 showed an increased sensitivity towards tetracycline and its derivatives as shown in in FIG. 3 of Urlinger et al., 2000 and functions at 10 fold lower concentrations than rtTA. In some embodiments, the improved variant of rtTA is rtTA-M2 from of Urlinger et al., 2000. Alternative improved variants are described in Table 1 of Zhou et al., 2006 (Zhou, X. et al. (2006) ‘Optimization of the Tet-On system for regulated gene expression through viral evolution’, Gene Therapy, 13(19), pp. 1382-1390. doi: 10.1038/sj.gt.3302780), which is incorporated herein by reference. The majority of these variants were shown to have higher transcriptional activity and doxycycline sensitivity than rtTA as described in FIG. 3 of Zhou et al., 2006. The highest performing variants were seven-fold more active and 100 times more sensitive to doxycycline. In some embodiments, the improved variant of rtTA is V14, V15 or V16 from Zhou et al., 2006.

One commercially available tetracycline-inducible system is the T-Rex system from Life-Technologies (see e.g. Life-Technologies (2014) ‘Inducible Protein Expression Using the T-REx™ System’, 1, pp. 1-12. Available at:

www.lifetechnologies.com/de/de/home/references/protocols/proteins-expression-isolation-and-analysis/protein-expression-protocol/inducible-protein-expression-using-the-trex-system.reg.us.html/).

The inducible promoter may be induced by absence of a molecule and presence of a different molecule. One example is with induction with tetracycline absence and estrogen presence. In some embodiments, the inducible promoter may be induced by removal of tetracycline and addition of estrogen as described in Iida et al., 1996 (Iida, A. et al. (1996) ‘Inducible gene expression by retrovirus-mediated transfer of a modified tetracycline-regulated system.’, Journal of Virology, 70(9), pp. 6054-6059. doi: 10.1128/jvi.70.9.6054-6059.1996), which is incorporated herein by reference. This specific inducibility was achieved by the addition of the ligand-binding domain of the estrogen receptor to the carboxy terminal of the tTA transactivator. Such modified transactivator was shown result in high expression of the gene of interest in the absence of tetracycline and the presence of estrogen as shown in FIG. 3 of Iida et al., 1996.

The inducible promoter may be induced by small molecule enhancers. Suitable promoters induced by small molecule enhancers such as aromatic carboxylic acids, hydroxamic acids and acetamides are described in Allen et al. (Biotechnol. Bioeng. 2008; 100: 1193-1204), which is incorporated herein by reference.

The inducible promoter may be induced by a synthetic steroid. In some embodiments, the inducible promoter may be induced by mifepristone, also known as RU-486. A hybrid mifespristone-responsive transcription factor, LexPR transactivator, was created by Emelyanov and Parinov, 2008 (Emelyanov, A. and Parinov, S. (2008) ‘Mifepristone-inducible LexPR system to drive and control gene expression in transgenic zebrafish’, Developmental Biology, 320(1), pp. 113-121. doi: 10.1016/j.ydbio.2008.04.042, which is incorporated herein by reference) by fusion of the DNA-binding domain of the bacterial LexA repressor, a truncated ligand-binding domain of the human progesterone receptor and the activation domain of the human NF-kB/p65 protein. Upon addition of mifepristone, LexPR induces expression from a promoter sequence harboring LexA binding sites as shown in FIG. 1 and FIG. 2 of Emelyanov and Parinov, 2008. Suitable commercially available mifepristone-inducible system is the GeneSwitch System (see e.g., Fisher, T. (1994) ‘Inducible Protein Expression Using GeneSwitch™ Technology’, pp. 1-25).

In some embodiments, the inducible promoter may be induced by the presence or the absence of cumate. In the cumate switch system from Mullick et al., 2006 (Mullick, A. et al. (2006) ‘The cumate gene-switch: A system for regulated expression in mammalian cells’, BMC Biotechnology, 6, pp. 1-18. doi: 10.1186/1472-6750-6-43, which is incorporated herein by reference), a repressor CymR blocks transcription from a promoter comprising CuO sequence placed downstream of the promoter. Once cumate is added, the CymR repressor is unable to bind to CuO and transcription from a promoter comprising CuO can proceed. This is shown in FIG. 1B and FIG. 2 from Mullick et al., 2006.

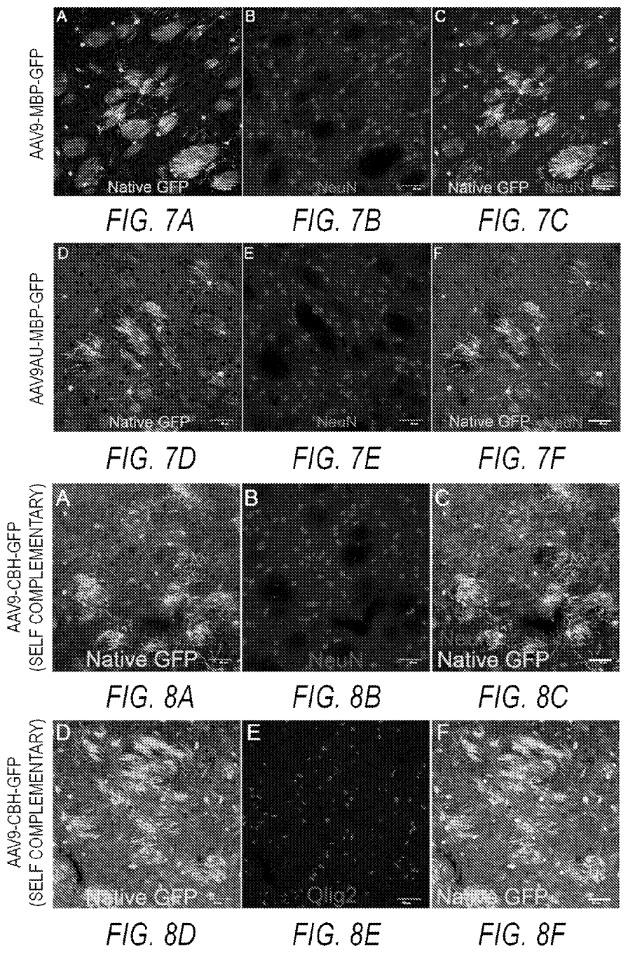

In an alternative cumate switch system, a chimeric transactivator (cTA) created from the fusion of CymR with the activation domain of VP16 does not prevent transcription from a promoter comprising CuO sequence upstream of a promoter in the presence of cumate. In the absence of cumate, the chimeric transactivator (cTA) binds to the CuO sequence and prevents transcription. This is shown in FIG. 1C and FIG. 3 from Mullick et al., 2006. In a third configuration, a reverse chimeric transactivator (rcTA) prevents transcription from a promoter comprising CuO sequence upstream of a promoter in the absence of cumate. In the presence of cumate, the rcTA binds to the CuO sequence and transcription from the promoter comprising CuO sequence can proceed. This is shown in FIG. 1D and FIG. 7 from Mullick et al., 2006. Suitable commercially available cumate-inducible systems is found from SBI Biosciences (see SBI (2020) ‘Cumate-inducible Systems For the ultimate in gene expression control, use SBI's cumate-CLIMATE-INDUCIBLE SYSTEMS’, pp. 1-13, which is incorporated herein by reference).

The inducible promoter may be induced by 4-hydroxytamoxifen (OHT) (referred to herein as 4-hydroxytamoxifen (OHT)-inducible promoters). Suitable 4-hydroxytamoxifen inducible promoters are described by Feil et al. (Biochemical and Biophysical Research Communications Volume 237, Issue 3, 28 Aug. 1997, Pages 752), which is incorporated herein by reference.

The inducible promoter may be a gas-inducible promoter, e.g., acetaldehyde-inducible. Examples of gas-inducible promoters are described in Weber et al., 2004 (Weber, W. et al. (2004) ‘Gas-inducible transgene expression in mammalian cells and mice’, Nature Biotechnology, 22(11), pp. 1440-1444. doi: 10.1038/nbt1021), which is incorporated herein by reference. The native acetaldehyde-inducible AlcR-PalcA system from Aspergillus nidulans has been adapted for mammalian use by introducing an AlcR-specific operator module to a human minimal promoter, together called PAIR, as shown in FIG. 1A. When AlcR is constitutively expressed in the cell of interest, upon introduction of acetaldehyde, acetaldehyde binds to AlcR and, in turn, the gene of interest which is under the control of the PAIR promoter is expressed, as shown in FIG. 1C, FIG. 2 and FIG. 3. In the absence of acetaldehyde, there is no expression of the gene of interest.

The inducible promoter may be induced by the presence or absence of a ribozyme (referred to herein as Riboswitch, Ribozyme and Aptazyme-Inducible Promoters). The ribozyme can, in turn be, be induced by a ligand.

The inducible promoter may be induced in the absence of a metabolite. In some embodiments, the metabolite may be glucosamine-6-phosphate-responsive. An example of a ribozyme which acts as a glucosamine-6-phosphate-responsive gene repressor is described by Winkler et al., 2004 (Winkler, W. C. et al. (2004) ‘Control of gene expression by a natural metabolite-responsive ribozyme’, Nature, 428(6980), pp. 281-286. doi: 10.1038/nature02362), which is incorporated herein by reference. The ribozyme is activated by glucosamine-6-phosphate in a concentration dependent manner as shown in FIG. 2C and cleaves the messenger RNA of the glmS gene. Upon modification, it is possible that this natural system may be applied to control of a gene of interest other than the glmS gene.

Other inducible promoters include, without limitation, metallothionein-inducible promoters, many of which have been described in the literature. See for example Shinichiro Takahashi “Positive and negative regulators of the metallothionein gene” Molecular Medicine Reports Mar. 9, 2015, P795-799, which is incorporated herein by reference.

The inducible promoter may be induced by a small molecule drug such as rapamycin (referred to herein as rapamycin-inducible promoters). A humanized system for pharmacologic control of gene expression using rapamycin is described in Rivera et al., 1996 (Rivera et al. Nature Medicine volume 2, pages 1028-1032(1996)), which is incorporated herein by reference. The natural ability of rapamycin to bind to FKBP12 and, in turn, for this complex to bind to FRAP was used by Rivera et al., 1996 to induce rapamycin-specific expression of a gene of interest. This was achieved by fusing one of the FKBP12/FRAP proteins to a DNA binding domain and the other protein to an activator domain. If the FKBP is fused with a DNA binding domain and FRAP is fused to an activator domain, there would be no transcription of the gene of interest in the absence of rapamycin since FKBP and FRAP do not interact, as shown in FIG. 1b. In the presence of rapamycin, FKBP and FRAP interact and the DNA binding domain and the activator domain are brought into close contact, resulting in transcription of the gene of interest as shown in FIG. 2 and FIG. 3.

The inducible promoter may be controlled by the chemically induced proximity (referred to herein as chemically-induced proximity-inducible promoters). Suitable small molecule-based systems for controlling protein abundance or activities are described in Liang et al. (Sci Signal. 2011 Mar. 15; 4(164):r52. doi: 10.1126/scisignal.2001449), which is incorporated herein by reference.

The inducible promoter may be induced by small synthetic molecules (referred to herein as Rheoswitch® inducible promoters). In some embodiments, these small synthetic molecules may be diacylhydrazine ligands. Suitable systems for inducible up- and down-regulation of gene expression is described in Cress et al. (Volume 66, Issue 8 Supplement, pp. 27) or Barrett et al. (Cancer Gene Therapy volume 25, pages 106-116(2018)), which are incorporated herein by reference. The RheoSwitch® system consists of two chimeric proteins derived from the ecdysone receptor (EcR) and RXR that are fused to a DNA-binding domain and an acidic transcriptional activation domain, respectively. The nuclear receptors can heterodimerize to create a functional transcription factor upon binding of a small molecule synthetic ligand and activate transcription from a responsive promoter linked to a gene of interest.

The invention also provides nucleic acids (e.g., isolated nucleic acids) encoding the modified AAV capsid protein or derivative thereof of the invention, described herein. The nucleic acid can be generated to encode an AAV capsid protein that has an amino acid sequence modification in one or more amino acids within the VP1/VP2 boundary that alters the permissiveness of an associated promoter when the promoter is within a target cell, as discussed herein. Vectors comprising the nucleic acid, and cells (in vivo or in culture) comprising the nucleic acids and/or vectors of the invention are also encompassed. Such nucleic acids, vectors and cells can be used, for example, as reagents (e.g., helper constructs or packaging cells) for the production of virus vectors as described herein.

In particular embodiments, the nucleic acid can be within a vector including but not limited to a plasmid, phage, viral vector (e.g., AAV vector, an adenovirus vector, a herpesvirus vector, or a baculovirus vector), bacterial artificial chromosome (BAC), or yeast artificial chromosome (YAC). For example, the nucleic acid can be in an AAV vector comprising a 5′ and/or 3′ terminal repeat (e.g., 5′ and/or 3′ AAV terminal repeat).

In some embodiments, the nucleic acid encoding the modified AAV capsid protein further comprises an AAV rep coding sequence. For example, the nucleic acid can be a helper construct for producing viral stocks.

In certain embodiments, the vector further comprises a promoter operably linked to a heterologous polynucleotide. In some embodiments, the promoter is a synthetic promoter. In some embodiments, the promoter is the CBA promoter. In some embodiments, the promoter is the CBh promoter. In some embodiments, the promoter is the synthetic JeTI promoter (Gray et al., Hum. Gene Ther. 22: 1143 (2011); Karumuthil-Melethil et al., Hum. Gene Ther. 27:509 (2016)). In some embodiments, the promoter is the human synapsin promoter.

An additional aspect of the invention relates to a cell in vitro comprising the nucleic acid that encodes the modified capsid protein described herein. In some embodiments, the nucleic acid is stably incorporated into the genome of the cell.

Another aspect of the invention relates to a virus particle comprising the nucleic acid that encodes the modified capsid protein described herein. The virus particle may be, without limitation, an AAV particle, an adenovirus particle, a herpesvirus particle, or a baculovirus particle.

Another aspect of the invention relates to an AAV capsid comprising the modified AAV capsid protein, described herein. Another aspect of the invention relates to a virus particle comprising the AAV capsid that comprises the modified AAV capsid protein discussed herein. The virus particle packages (i.e., encapsidates) a vector genome, optionally an AAV vector genome. In particular embodiments, the invention provides an AAV particle comprising an AAV capsid comprising the modified AAV capsid protein described herein, wherein the AAV capsid packages an AAV vector genome. In one embodiment, the AAV particle comprises an AAV capsid or AAV capsid protein encoded by the nucleic acid capsid coding sequences of the invention.

In particular embodiments, the particle that has a capsid comprising the altered capsid protein is a recombinant AAV vector comprising a heterologous nucleic acid of interest, e.g., for delivery to a cell. In some embodiments, the AAV vector genome comprises a promoter operably linked to the heterologous nucleic acid/nucleic acid of interest. In some embodiments, the promoter is a synthetic promoter. In some embodiments, the promoter is the CBA promoter. In some embodiments, the promoter is the CBh promoter. In some embodiments, the promoter is the JeTI promoter, also referred to as the UsP (Karumuthil-Melethil et al., Hum. Gene Ther. 27:509 (2016)). In some embodiments the promoter is a hSYN1 promoter. In some embodiments, the promoter is a CMB promoter. The viral particle is useful for the delivery of nucleic acids to cells in vitro, ex vivo, and in vivo. In representative embodiments, the recombinant vector of the invention can be advantageously employed to deliver or transfer nucleic acids to animal (e.g., mammalian) cells.

The heterologous nucleic acid may encode any protein or functional nucleic acid of interest. In some embodiments, the heterologous nucleic acid encodes a functional nucleic acid, e.g., an antisense RNA, microRNA, or RNAi. In some embodiments, the heterologous nucleic acid encodes a polypeptide, e.g., a therapeutic polypeptide or a reporter protein.

Nucleic acids of interest include nucleic acids encoding polypeptides, optionally therapeutic (e.g., for medical or veterinary uses) and/or immunogenic (e.g., for vaccines) polypeptides.

Therapeutic polypeptides include, but are not limited to, cystic fibrosis transmembrane regulator protein (CFTR), dystrophin (including the protein product of dystrophin mini-genes or micro-genes, see, e.g., Vincent et al., (1993) Nature Genetics 5:130; U.S. Patent Publication No. 2003017131; Wang et al., (2000) Proc. Natl. Acad. Sci. USA 97:13714-9 [mini-dystrophin]; Harper et al., (2002) Nature Med. 8:253-61 [micro-dystrophin]); mini-agrin, a laminin-α2, a sarcoglycan (α, β, γ or δ), Fukutin-related protein, myostatin pro-peptide, follistatin, dominant negative myostatin, an angiogenic factor (e.g., VEGF, angiopoietin-1 or 2), an anti-apoptotic factor (e.g., heme-oxygenase-1, TGF-β, inhibitors of pro-apoptotic signals such as caspases, proteases, kinases, death receptors [e.g., CD-095], modulators of cytochrome C release, inhibitors of mitochondrial pore opening and swelling); activin type II soluble receptor, anti-inflammatory polypeptides such as the Ikappa B dominant mutant, sarcospan, utrophin, mini-utrophin, antibodies or antibody fragments against myostatin or myostatin propeptide, cell cycle modulators, Rho kinase modulators such as Cethrin, which is a modified bacterial C3 exoenzyme [available from BioAxone Therapeutics, Inc., Saint-Lauren, Quebec, Canada], BCL-xL, BCL2, XIAP, FLICEc-s, dominant-negative caspase-8, dominant negative caspase-9, SPI-6 (see, e.g., U.S. Patent Application No. 20070026076), transcriptional factor PGC-α1, Pinch gene, ILK gene and thymosin β4 gene), clotting factors (e.g., Factor VIII, Factor IX, Factor X, etc.), erythropoietin, angiostatin, endostatin, catalase, tyrosine hydroxylase, an intracellular and/or extracellular superoxide dismutase, leptin, the LDL receptor, neprilysin, lipoprotein lipase, ornithine transcarbamylase, β-globin, α-globin, spectrin, α₁-antitrypsin, methyl cytosine binding protein 2, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, a cytokine (e.g., α-interferon, β-interferon, interferon-γ, interleukins-1 through -14, granulocyte-macrophage colony stimulating factor, lymphotoxin, and the like), peptide growth factors, neurotrophic factors and hormones (e.g., somatotropin, insulin, insulin-like growth factors including IGF-1 and IGF-2, GLP-1, platelet derived growth factor, epidermal growth factor, fibroblast growth factor, nerve growth factor, neurotrophic factor-3 and -4, brain-derived neurotrophic factor, glial derived growth factor, transforming growth factor-α and -β, and the like), bone morphogenic proteins (including RANKL and VEGF), a lysosomal protein, a glutamate receptor, a lymphokine, soluble CD4, an Fc receptor, a T cell receptor, ApoE, ApoC, inhibitor 1 of protein phosphatase inhibitor 1 (I-1), phospholamban, serca2a, lysosomal acid α-glucosidase, α-galactosidase A, Barkct, β2-adrenergic receptor, β2-adrenergic receptor kinase (BARK), phosphoinositide-3 kinase (PI3 kinase), calsarcin, a receptor (e.g., the tumor necrosis growth factor-α soluble receptor), an anti-inflammatory factor such as TRAP, Pim-1, PGC-1α, SOD-1, SOD-2, ECF-SOD, kallikrein, thymosin-β4, hypoxia-inducible transcription factor [HIF], an angiogenic factor, S100A1, parvalbumin, adenylyl cyclase type 6, a molecule that effects G-protein coupled receptor kinase type 2 knockdown such as a truncated constitutively active bARKct; phospholamban inhibitory or dominant-negative molecules such as phospholamban S16E, a monoclonal antibody (including single chain monoclonal antibodies) or a suicide gene product (e.g., thymidine kinase, cytosine deaminase, diphtheria toxin, and tumor necrosis factors such as TNF-α), and any other polypeptide that has a therapeutic effect in a subject in need thereof.

Heterologous nucleic acids/nucleic acids of interest encoding polypeptides include those encoding reporter polypeptides (e.g., an enzyme). Reporter polypeptides are known in the art and include, but are not limited to, a fluorescent protein (e.g., EGFP, GFP, RFP, BFP, YFP, or dsRED2), an enzyme that produces a detectable product, such as luciferase (e.g., from Gaussia, Renilla, or Photinus), β-galactosidase, β-glucuronidase, alkaline phosphatase, and chloramphenicol acetyltransferase gene, or proteins that can be directly detected. Virtually any protein can be directly detected by using, for example, specific antibodies to the protein. Additional markers (and associated antibiotics) that are suitable for either positive or negative selection of eukaryotic cells are disclosed in Sambrook and Russell (2001), Molecular Cloning, 3rd Ed., Cold Spring Harbor Laboratory Press, Cold Spring Harbor, N.Y., and Ausubel et al. (1992), Current Protocols in Molecular Biology, John Wiley & Sons, including periodic updates.

Alternatively, the heterologous nucleic acid/nucleic acid of interest may encode a functional RNA, e.g., an antisense oligonucleotide, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including small interfering RNAs (siRNA) that mediate gene silencing (see, Sharp et al., (2000) Science 287:2431), microRNA, or other non-translated “functional” RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi or antisense RNA against the multiple drug resistance (MDR) gene product (e.g., to treat tumors and/or for administration to the heart to prevent damage by chemotherapy), RNAi or antisense RNA against myostatin (Duchenne or Becker muscular dystrophy), RNAi or antisense RNA against VEGF or a tumor immunogen including but not limited to those tumor immunogens specifically described herein (to treat tumors), RNAi or antisense oligonucleotides targeting mutated dystrophins (Duchenne or Becker muscular dystrophy), RNAi or antisense RNA against the hepatitis B surface antigen gene (to prevent and/or treat hepatitis B infection), RNAi or antisense RNA against the HIV tat and/or rev genes (to prevent and/or treat HIV) and/or RNAi or antisense RNA against any other immunogen from a pathogen (to protect a subject from the pathogen) or a defective gene product (to prevent or treat disease). RNAi or antisense RNA against the targets described above or any other target can also be employed as a research reagent.

As is known in the art, antisense nucleic acids (e.g., DNA or RNA) and inhibitory RNA (e.g., microRNA and RNAi such as siRNA or shRNA) sequences can be used to induce “exon skipping” in patients with muscular dystrophy arising from defects in the dystrophin gene. Thus, the heterologous nucleic acid can encode an antisense nucleic acid or inhibitory RNA that induces appropriate exon skipping. Those skilled in the art will appreciate that the particular approach to exon skipping depends upon the nature of the underlying defect in the dystrophin gene, and numerous such strategies are known in the art. Exemplary antisense nucleic acids and inhibitory RNA sequences target the upstream branch point and/or downstream donor splice site and/or internal splicing enhancer sequence of one or more of the dystrophin exons (e.g., exons 19 or 23). For example, in particular embodiments, the heterologous nucleic acid/nucleic acid of interest encodes an antisense nucleic acid or inhibitory RNA directed against the upstream branch point and downstream splice donor site of exon 19 or 23 of the dystrophin gene. Such sequences can be incorporated into an AAV vector delivering a modified U7 snRNA and the antisense nucleic acid or inhibitory RNA (see, e.g., Goyenvalle et al., (2004) Science 306:1796-1799). As another strategy, a modified U1 snRNA can be incorporated into an AAV vector along with siRNA, microRNA or antisense RNA complementary to the upstream and downstream splice sites of a dystrophin exon (e.g., exon 19 or 23) (see, e.g., Denti et al., (2006) Proc. Nat. Acad. Sci. USA 103:3758-3763). Further, antisense nucleic acids and inhibitory RNA can target the splicing enhancer sequences within exons 19, 43, 45 or 53 (see, e.g., U.S. Pat. Nos. 6,653,467; 6,727,355; and 6,653,466).

The recombinant virus vector may also comprise a heterologous nucleotide sequence that shares homology with and recombines with a locus on the host chromosome. This approach may be utilized to correct a genetic defect in the host cell.

The present invention also provides recombinant virus vectors that express an immunogenic polypeptide, e.g., for vaccination. The heterologous nucleic acid may encode any immunogen of interest known in the art including, but are not limited to, immunogens from human immunodeficiency virus, influenza virus, gag proteins, tumor antigens, cancer antigens, bacterial antigens, viral antigens, and the like. Alternatively, the immunogen can be presented in the virus capsid (e.g., incorporated therein) or tethered to the virus capsid (e.g., by covalent modification).

The use of parvoviruses as vaccines is known in the art (see, e.g., Miyamura et al., (1994) Proc. Nat. Acad. Sci. USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.; the disclosures of which are incorporated herein in their entireties by reference). The antigen may be presented in the virus capsid. Alternatively, the antigen may be expressed from a heterologous nucleic acid introduced into a recombinant vector genome.

An immunogenic polypeptide, or immunogen, may be any polypeptide suitable for protecting the subject against a disease, including but not limited to microbial, bacterial, protozoal, parasitic, fungal and viral diseases. For example, the immunogen may be an orthomyxovirus immunogen (e.g., an influenza virus immunogen, such as the influenza virus hemagglutinin (HA) surface protein or the influenza virus nucleoprotein gene, or an equine influenza virus immunogen), or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogen may also be an arenavirus immunogen (e.g., Lassa fever virus immunogen, such as the Lassa fever virus nucleocapsid protein gene and the Lassa fever envelope glycoprotein gene), a poxvirus immunogen (e.g., vaccinia, such as the vaccinia L1 or L8 genes), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP genes), a bunyavirus immunogen (e.g., RVFV, CCHF, and SFS viruses), or a coronavirus immunogen (e.g., an infectious human coronavirus immunogen, such as the human coronavirus envelope glycoprotein gene, or a porcine transmissible gastroenteritis virus immunogen, or an avian infectious bronchitis virus immunogen, or a severe acute respiratory syndrome (SARS) immunogen such as a S [S1 or S2], M, E, or N protein or an immunogenic fragment thereof). The immunogen may further be a polio immunogen, herpes immunogen (e.g., CMV, EBV, HSV immunogens) mumps immunogen, measles immunogen, rubella immunogen, diphtheria toxin or other diphtheria immunogen, pertussis antigen, hepatitis (e.g., hepatitis A, hepatitis B or hepatitis C) immunogen, or any other vaccine immunogen known in the art.

Alternatively, the immunogen may be any tumor or cancer cell antigen. Optionally, the tumor or cancer antigen is expressed on the surface of the cancer cell. Exemplary cancer and tumor cell antigens are described in S. A. Rosenberg, (1999) Immunity 10:281). Illustrative cancer and tumor antigens include, but are not limited to: BRCA1 gene product, BRCA2 gene product, gp100, tyrosinase, GAGE-1/2, BAGE, RAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J Exp. Med., 180:347; Kawakami et al., (1994) Cancer Res. 54:3124) including MART-1 (Coulie et al., (1991) J. Exp. Med. 180:35), gp100 (Wick et al., (1988) J Cutan. Pathol. 4:201) and MAGE antigen (MAGE-1, MAGE-2 and MAGE-3) (Van der Bruggen et al., (1991) Science, 254:1643), CEA, TRP-1; TRP-2; P-15 and tyrosinase (Brichard et al., (1993) J. Exp. Med. 178:489); HER-2/neu gene product (U.S. Pat. No. 4,968,603); CA 125; HE4; LK26; FB5 (endosialin); TAG 72; AFP; CA19-9; NSE; DU-PAN-2; CA50; Span-1; CA72-4; HCG; STN (sialyl Tn antigen); c-erbB-2 proteins; PSA; L-CanAg; estrogen receptor; milk fat globulin; p53 tumor suppressor protein (Levine, (1993) Ann. Rev. Biochem. 62:623); mucin antigens (international patent publication WO 90/05142); telomerases; nuclear matrix proteins; prostatic acid phosphatase; papilloma virus antigens; and antigens associated with the following cancers: melanomas, adenocarcinoma, thymoma, sarcoma, lung cancer, liver cancer, colorectal cancer, non-Hodgkin's lymphoma, Hodgkin's lymphoma, leukemias, uterine cancer, breast cancer, prostate cancer, ovarian cancer, cervical cancer, bladder cancer, kidney cancer, pancreatic cancer, brain cancer, kidney cancer, stomach cancer, esophageal cancer, head and neck cancer and others (see, e.g., Rosenberg, (1996) Annu. Rev. Med. 47:481-91).

Alternatively, the heterologous nucleotide sequence may encode any polypeptide that is desirably produced in a cell in vitro, ex vivo, or in vivo. For example, the virus vectors may be introduced into cultured cells and the expressed protein product isolated therefrom.

It will be understood by those skilled in the art that the heterologous nucleic acid(s) of interest may be operably associated with appropriate control sequences. For example, the heterologous nucleic acid may be operably associated with expression control elements, such as transcription/translation control signals, origins of replication, polyadenylation signals, internal ribosome entry sites (IRES), promoters, enhancers, and the like.

Those skilled in the art will further appreciate that a variety of promoter/enhancer elements may be used depending on the level and tissue-specific expression desired. The promoter/enhancer may be constitutive or inducible, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced.

Promoter/enhancer elements can be native to the target cell or subject to be treated and/or native to the heterologous nucleic acid sequence. The promoter/enhancer element is generally chosen so that it will function in the target cell(s) of interest. In representative embodiments, the promoter/enhancer element is a mammalian promoter/enhancer element. The promoter/enhance element may be an RNA polymerase II-based promoter or an RNA polymerase III-based promoter. The promoter/enhance element may be constitutive or inducible.

Inducible expression control elements are generally used in those applications in which it is desirable to provide regulation over expression of the heterologous nucleic acid sequence(s). Inducible promoters/enhancer elements for gene delivery can be tissue-specific or tissue-preferred promoter/enhancer elements, and include muscle specific or preferred (including cardiac, skeletal and/or smooth muscle), neural tissue specific or preferred (including brain-specific), eye (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally employed for efficient translation of inserted protein coding sequences. These exogenous translational control sequences, which may include the ATG initiation codon and adjacent sequences, can be of a variety of origins, both natural and synthetic.

Aspects of the invention also relate to synthetic AAV particles comprising a modified AAV capsid protein described herein and an AAV genome, wherein the AAV genome encodes the AAV capsid. Collections or libraries of such chimeric AAV particles, wherein the collection or library comprises 2 or more, 10 or more, 50 or more, 100 or more, 1000 or more, 10⁴ or more, 10⁵ or more, or 10⁶ or more distinct sequences, are also encompassed.

The present invention further encompasses “empty” capsid particles (i.e., in the absence of a vector genome) comprising, consisting of, or consisting essentially of the AAV capsid protein or derivative thereof of the invention. The empty capsid particle will comprise the AAV capsid protein that has an amino acid sequence modification in one or more amino acids within the VP1/VP2 boundary that alters the permissiveness of an associated promoter when the promoter is within a target cell.

The synthetic AAV capsids of the invention can be used as “capsid vehicles,” as has been described in U.S. Pat. No. 5,863,541. Molecules that can be covalently linked, bound to or packaged by the virus capsids and transferred into a cell include DNA, RNA, a lipid, a carbohydrate, a polypeptide, a small organic molecule, or combinations of the same. Further, molecules can be associated with (e.g., “tethered to”) the outside of the virus capsid for transfer of the molecules into host target cells. In one embodiment of the invention the molecule is covalently linked (i.e., conjugated or chemically coupled) to the capsid proteins. Methods of covalently linking molecules are known by those skilled in the art.

The virus capsids of the invention also find use in raising antibodies against the novel capsid structures. As a further alternative, an exogenous amino acid sequence may be inserted into the virus capsid for antigen presentation to a cell, e.g., for administration to a subject to produce an immune response to the exogenous amino acid sequence.

The invention also relates to packaging cells stably comprising a nucleic acid of the invention. For example, the nucleic acid can be stably incorporated into the genome of the cell or can be stably maintained in an episomal form (e.g., an “EBV based nuclear episome”).

The nucleic acid can be incorporated into a delivery vector, such as a viral delivery vector. To illustrate, the nucleic acid of the invention can be packaged in an AAV particle, an adenovirus particle, a herpesvirus particle, a baculovirus particle, or any other suitable virus particle.

Moreover, the nucleic acid can be operably associated with a promoter element. Promoter elements are described in more detail herein.

Other aspects of the invention relate to methods of producing the virus vectors of the invention. In some embodiments, the method is a method of producing a recombinant virus vector with the capsid protein comprising the amino acid sequence modification at the VP1/VP2 boundary described herein. The method comprises providing to a cell in vitro, (a) a template comprising (i) a heterologous nucleic acid, and (ii) packaging signal sequences sufficient for the encapsidation of the AAV template into virus particles (e.g., one or more (e.g., two) terminal repeats, such as AAV terminal repeats), and (b) AAV sequences sufficient for replication and encapsidation of the template into viral particles (e.g., the AAV rep and AAV cap sequences encoding an AAV capsid proteins comprising the amino acid sequence modification at the VP1/VP2 boundary that alter permissiveness of a promoter operably linked to the heterologous nucleic acid as described herein). The template and AAV replication and capsid sequences are provided under conditions such that recombinant virus particles comprising the template packaged within the capsid are produced in the cell. The method can further comprise the step of collecting the virus particles from the cell. Virus particles may be collected from the medium and/or by lysing the cells.

One aspect of the invention is a method of producing a rAAV particle comprising a modified AAV capsid described herein. The method comprises providing a cell in vitro with a nucleic acid encoding an AAV capsid proteins comprising the amino acid sequence modification at the VP1/VP2 boundary that alter permissiveness of a promoter, an AAV rep coding sequence, an AAV vector genome comprising a heterologous nucleic acid operably linked to a promoter, and helper functions for generating a productive AAV infection; and allowing assembly of the rAAV particles comprising the AAV capsid and encapsidating the rAAV vector genome.

The cell is typically a cell that is permissive for AAV viral replication. Any suitable cell known in the art may be employed, such as mammalian cells. Also suitable are trans-complementing packaging cell lines that provide functions deleted from a replication-defective helper virus, e.g., 293 cells or other Ela trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an EBV based nuclear episome.

As a further alternative, the rep/cap sequences may be stably carried (episomal or integrated) within a cell.

Typically, the AAV rep/cap sequences will not be flanked by the AAV packaging sequences (e.g., AAV ITRs), to prevent rescue and/or packaging of these sequences.

The template (e.g., an rAAV vector genome) can be provided to the cell using any method known in the art. For example, the template may be supplied by a non-viral (e.g., plasmid) or viral vector. In particular embodiments, the template is supplied by a herpesvirus or adenovirus vector (e.g., inserted into the E1a or E3 regions of a deleted adenovirus). As another illustration, Palombo et al., (1998) J. Virol. 72:5025, describe a baculovirus vector carrying a reporter gene flanked by the AAV ITRs. EBV vectors may also be employed to deliver the template, as described above with respect to the rep/cap genes.

In another representative embodiment, the template is provided by a replicating rAAV virus. In still other embodiments, an AAV provirus is stably integrated into the chromosome of the cell.

To obtain maximal virus titers, helper virus functions (e.g., adenovirus or herpesvirus) essential for a productive AAV infection are generally provided to the cell. Helper virus sequences necessary for AAV replication are known in the art. Typically, these sequences are provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes required for efficient AAV production as described by Ferrari et al., (1997) Nature Med. 3:1295, and U.S. Pat. Nos. 6,040,183 and 6,093,570.

Further, the helper virus functions may be provided by a packaging cell with the helper genes integrated in the chromosome or maintained as a stable extrachromosomal element. In representative embodiments, the helper virus sequences cannot be packaged into AAV virions, e.g., are not flanked by AAV ITRs.

Those skilled in the art will appreciate that it may be advantageous to provide the AAV replication and capsid sequences and the helper virus sequences (e.g., adenovirus sequences) on a single helper construct. This helper construct may be a non-viral or viral construct, but is optionally a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector further contains the rAAV template. The AAV rep/cap sequences and/or the rAAV template may be inserted into a deleted region (e.g., the Ela or E3 regions) of the adenovirus.

In a further embodiment, the AAV rep/cap sequences and the adenovirus helper sequences are supplied by a single adenovirus helper vector. The rAAV template is provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as a “EBV based nuclear episome,” see Margolski, (1992) Curr. Top. Microbiol. Immun. 158:67).

In a further exemplary embodiment, the AAV rep/cap sequences and adenovirus helper sequences are provided by a single adenovirus helper. The rAAV template is provided as a separate replicating viral vector. For example, the rAAV template may be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, in representative embodiments, the adenovirus helper sequences and the AAV rep/cap sequences are not flanked by the AAV packaging sequences (e.g., the AAV ITRs), so that these sequences are not packaged into the AAV virions.

Herpesvirus may also be used as a helper virus in AAV packaging methods. Hybrid herpesviruses encoding the AAV rep protein(s) may advantageously facilitate for more scalable AAV vector production schemes. A hybrid herpes simplex virus type I (HSV-1) vector expressing the AAV-2 rep and cap genes has been described (Conway et al., (1999) Gene Therapy 6:986 and WO 00/17377, the disclosures of which are incorporated herein in their entireties).

As a further alternative, the virus vectors of the invention can be produced in insect cells using baculovirus vectors to deliver the rep/cap genes and rAAV template as described by Urabe et al., (2002) Human Gene Therapy 13:1935-43.

Other methods of producing AAV use stably transformed packaging cells (see, e.g., U.S. Pat. No. 5,658,785).

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al., (1999) Gene Therapy 6:973). In representative embodiments, deleted replication-defective helper viruses are used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

The inventive packaging methods may be employed to produce high titer stocks of virus particles. In particular embodiments, the virus stock has a titer of at least about 10⁵ transducing units (tu)/ml, at least about 10⁶ tu/ml, at least about 10⁷ tu/ml, at least about 10⁸ tu/ml, at least about 10⁹ tu/ml, or at least about 10¹⁰ tu/ml.

The novel capsid protein and capsid structures find use in raising antibodies, for example, for diagnostic or therapeutic uses or as a research reagent. Thus, the invention also provides antibodies against the novel capsid proteins and capsids of the invention.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, goat, sheep or human, or can be a chimeric antibody. See, e.g., Walker et al., Mol. Immunol. 26, 403-11 (1989). The antibodies can be recombinant monoclonal antibodies, for example, produced according to the methods disclosed in U.S. Pat. Nos. 4,474,893 or 4,816,567. The antibodies can also be chemically constructed, for example, according to the method disclosed in U.S. Pat. No. 4,676,980.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254, 1275-1281).

Polyclonal antibodies can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies can be produced in a hybridoma cell line according to the technique of Kohler and Milstein, (1975) Nature 265, 495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., W. Huse, (1989) Science 246, 1275-81.

Antibodies specific to a target polypeptide can also be obtained by phage display techniques known in the art.

Various immunoassays can be used for screening to identify antibodies having the desired specificity. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes can be used as well as a competitive binding assay.

Antibodies can be conjugated to a solid support (e.g., beads, plates, slides or wells formed from materials such as latex or polystyrene) in accordance with known techniques. Antibodies can likewise be directly or indirectly conjugated to detectable groups such as radiolabels (e.g. ³⁵S, ¹²⁵I, ¹³¹I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

### Methods of Using Modified AAV Capsids

The present invention also relates to methods for delivering a heterologous nucleic acid/nucleic acid of interest to a subject. The virus vectors that comprise the modified AAV capsid proteins discussed herein may be employed to deliver a nucleotide sequence of interest to a cell in vitro, e.g., to produce a polypeptide or nucleic acid in vitro or for ex vivo gene therapy. The vectors are additionally useful in a method of delivering a nucleotide sequence to a subject in need thereof, e.g., to express a therapeutic or immunogenic polypeptide or nucleic acid. In this manner, the polypeptide or nucleic acid may thus be produced in vivo in the subject. The subject may be in need of the polypeptide or nucleic acid because the subject has a deficiency of the polypeptide, or because the production of the polypeptide or nucleic acid in the subject may impart some therapeutic effect, as a method of treatment or otherwise, and as explained further below.

In particular embodiments, the vectors are useful to express a polypeptide or nucleic acid that provides a beneficial effect to the subject in general. In other embodiments, the vectors are useful to express a polypeptide or nucleic acid that provides a beneficial effect to cells in the subject.

Thus, one aspect of the invention relates to a method of delivering a nucleic acid of interest to a cell, e.g., a CNS cell, the method comprising contacting the cell with the AAV particle comprising the modified capsid protein described herein, that comprises the nucleic acid of interest within its genome operably linked to a promoter (e.g., a promoter described herein).

In another aspect, the invention relates to a method of delivering a nucleic acid of interest to a cell, e.g., a CNS cell, in a mammalian subject, the method comprising administering an effective amount of the AAV particle that comprises the modified capsid protein described herein, and the nucleic acid of interest within its genome operably linked to a promoter, or pharmaceutical formulation of the invention to a mammalian subject, thereby delivering the nucleic acid of interest to a cell in the mammalian subject.

A further aspect of the invention relates to a method of treating a disorder in a mammalian subject in need thereof, wherein the disorder is treatable by expressing a product in the subject, the method comprising administering a therapeutically effective amount of the AAV particle that comprises the modified AAV capsid protein described herein, and further comprises a nucleic acid that encodes the product within its genome operably linked to a promoter, to the subject, wherein the product is expressed, thereby treating the disorder. In some embodiments, the disorder is treatable by expressing an encoded therapeutic product in the CNS of the subject, and the AAV particle is delivered to the CNS of the subject.

In particular embodiments, the AAV particle may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The AAV particle of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of an AAV particle encoding one or more neurotrophic factors.

Age-related macular degeneration involves both angiogenesis and retinal degeneration. This disorder can be treated by administering an AAV particle encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the heterologous agent. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence, or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using an AAV particle of the invention to treat a pituitary tumor. According to this embodiment, the AAV particle encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art.

In particular embodiments, the AAV particle can comprise a secretory signal as described in U.S. Pat. No. 7,071,172.

In representative embodiments of the invention, the AAV particle or pharmaceutical formulation containing the AAV particle is administered to the CNS (e.g., to the brain or to the eye). The AAV particle or formulation may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The AAV particle also be administered to different regions of the eye such as the retina, cornea and/or optic nerve.

The AAV particle may be delivered into the cerebrospinal fluid (e.g., by lumbar puncture) for more disperse administration of the AAV particle. The AAV particle may further be administered intravascularly to the CNS in situations in which the blood-brain barrier has been perturbed (e.g., brain tumor or cerebral infarct).

The AAV particle can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the AAV particle is administered in a liquid formulation by direct injection (e.g., stereotactic injection) to the desired region or compartment in the CNS. In other embodiments, the AAV particle may be provided by topical application to the desired region or by intra-nasal administration of an aerosol formulation. Administration to the eye, may be by topical application of liquid droplets. As a further alternative, the AAV particle may be administered as a solid, slow-release formulation (see, e.g., U.S. Pat. No. 7,201,898).

In yet additional embodiments, the AAV particle that comprises the modified capsid proteins described herein can used for retrograde transport to treat and/or prevent diseases and disorders involving motor neurons (e.g., amyotrophic lateral sclerosis (ALS); spinal muscular atrophy (SMA), etc.). For example, the AAV particle can be delivered to muscle tissue from which it can migrate into neurons.

In certain embodiments, the AAV particle is administered to a subject in need thereof as early as possible in the life of the subject, e.g., as soon as the subject is diagnosed with a disease or disorder. In some embodiments, the methods are carried out on a newborn subject, e.g., after newborn screening has identified a disease or disorder. In some embodiments, methods are carried out on a subject prior to the age of 10 years, e.g., prior to 1, 2, 3, 4, 5, 6, 7, 8, 9, or 10 years of age. In some embodiments, the methods are carried out on juvenile or adult subjects after the age of 10 years. In some embodiments, the methods are carried out on a fetus in utero, e.g., after prenatal screening has identified a disease or disorder. In some embodiments, the methods are carried out on a subject as soon as the subject develops symptoms associated with a disease or disorder. In some embodiments, the methods are carried out on a subject before the subject develops symptoms associated with a disease or disorder, e.g., a subject that is suspected or diagnosed as having a disease or disorder but has not started to exhibit symptoms.

Another aspect of the invention relates to a method of altering expression of a heterologous polynucleotide present in an AAV vector in cells of the CNS, comprising preparing the AAV vector with the AAV capsid protein or chimeric capsid protein of the invention.

An additional aspect of the invention relates to a method for altering expression of a transgene operably linked to a promoter and delivered to a cell by a rAAV vector. The method involves modifying the amino acid sequence of at least one amino acid within the VP1/VP2 boundary of a capsid protein of the rAAV vector, wherein the amino acid sequence modification alters the permissiveness of the promoter within the cell, as discussed herein. The modification may be any modification at any location in the VP1/VP2 boundary as described herein.

In general, the virus vectors of the invention may be employed to deliver any foreign nucleic acid with a biological effect to treat or ameliorate the symptoms associated with any disorder related to gene expression. Further, the invention can be used to treat any disease state for which it is beneficial to deliver a therapeutic polypeptide. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (inhibitory RNA including without limitation RNAi such as siRNA or shRNA, antisense RNA or microRNA to remove repeats), amyotrophic lateral sclerosis, epilepsy (galanin, neurotrophic factors), and other neurological disorders, cancer (endostatin, angiostatin, TRAIL, FAS-ligand, cytokines including interferons; inhibitory RNA including without limitation RNAi (such as siRNA or shRNA), antisense RNA and microRNA including inhibitory RNA against VEGF, the multiple drug resistance gene product or a cancer immunogen), diabetes mellitus (insulin, PGC-α1, GLP-1, myostatin pro-peptide, glucose transporter 4), muscular dystrophies including Duchenne and Becker (e.g., dystrophin, mini-dystrophin, micro-dystrophin, insulin-like growth factor I, a sarcoglycan [e.g., α, β, γ], Inhibitory RNA [e.g., RNAi, antisense RNA or microRNA] against myostatin or myostatin propeptide, laminin-alpha2, Fukutin-related protein, dominant negative myostatin, follistatin, activin type II soluble receptor, anti-inflammatory polypeptides such as the Ikappa B dominant mutant, sarcospan, utrophin, mini-utrophin, inhibitory RNA [e.g., RNAi, antisense RNA or microRNA] against splice junctions in the dystrophin gene to induce exon skipping [see, e.g., WO/2003/095647], inhibitory RNA (e.g., RNAi, antisense RNA or micro RNA] against U7 snRNAs to induce exon skipping [see, e.g., WO/2006/021724], and antibodies or antibody fragments against myostatin or myostatin propeptide), Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects including other lysosomal storage disorders and glycogen storage disorders, congenital emphysema (al-antitrypsin), Lesch-Nyhan Syndrome (hypoxanthine guanine phosphoribosyl transferase), Niemann-Pick disease (sphingomyelinase), Maple Syrup Urine Disease (branched-chain keto acid dehydrogenase), retinal degenerative diseases (and other diseases of the eye and retina; e.g., PDGF, endostatin and/or angiostatin for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver (RNAi such as siRNA or shRNA, microRNA or antisense RNA for hepatitis B and/or hepatitis C genes), kidney, heart including congestive heart failure or peripheral artery disease (PAD) (e.g., by delivering protein phosphatase inhibitor I [I-1], phospholamban, sarcoplasmic endoreticulum Ca²⁺-ATPase [serca2a], zinc finger proteins that regulate the phospholamban gene, Pim-1, PGC-1α, SOD-1, SOD-2, ECF-SOD, kallikrein, thymosin-04, hypoxia-inducible transcription factor [HIF], βarkct, β2-adrenergic receptor, β2-adrenergic receptor kinase [βARK], phosphoinositide-3 kinase [PI3 kinase], calsarcin, an angiogenic factor, S100A1, parvalbumin, adenylyl cyclase type 6, a molecule that effects G-protein coupled receptor kinase type 2 knockdown such as a truncated constitutively active bARKct, an inhibitory RNA [e.g., RNAi, antisense RNA or microRNA] against phospholamban; phospholamban inhibitory or dominant-negative molecules such as phospholamban S16E, etc.), arthritis (insulin-like growth factors), joint disorders (insulin-like growth factors), intimal hyperplasia (e.g., by delivering enos, inos), improve survival of heart transplants (superoxide dismutase), AIDS (soluble CD4), muscle wasting (insulin-like growth factor I, myostatin pro-peptide, an anti-apoptotic factor, follistatin), limb ischemia (VEGF, FGF, PGC-1a, EC-SOD, HIF), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including RANKL and/or VEGF) can be administered with a bone allograft, for example, following a break or surgical removal in a cancer patient.

Exemplary lysosomal storage diseases that can be treated according to the present invention include without limitation: Hurler's Syndrome (MPS IH), Scheie's Syndrome (MPS IS), and Hurler-Scheie Syndrome (MPS IH/S) (α-L-iduronidase); Hunter's Syndrome (MPS II) (iduronate sulfate sulfatase); Sanfilippo A Syndrome (MPS IIIA) (Heparan-S-sulfate sulfaminidase), Sanfilippo B Syndrome (MPS IIIB) (N-acetyl-D-glucosaminidase), Sanfilippo C Syndrome (MPS IIIC) (Acetyl-CoA-glucosaminide N-acetyltransferase), Sanfilippo D Syndrome (MPS IIID) (N-acetyl-glucosaminine-6-sulfate sulfatase); Morquio A disease (MPS IVA) (Galactosamine-6-sulfate sulfatase), Morquio B disease (MPS IV B) (β-Galactosidase); Maroteaux-lmay disease (MPS VI) (arylsulfatase B); Sly Syndrome (MPS VII) (β-glucuronidase); hyaluronidase deficiency (MPS IX) (hyaluronidase); sialidosis (mucolipidosis I), mucolipidosis II (I-Cell disease) (N-actylglucos-aminyl-1-phosphotransferase catalytic subunit), mucolipidosis III (pseudo-Hurler polydystrophy) (N-acetylglucos-aminyl-1-phosphotransferase; type IIIA [catalytic subunit] and type IIIC [substrate recognition subunit]); GM1 gangliosidosis (ganglioside β-galactosidase), GM2 gangliosidosis Type I (Tay-Sachs disease) (β-hexaminidase A), GM2 gangliosidosis type II (Sandhoff's disease) (β-hexosaminidase B); Niemann-Pick disease (Types A and B) (sphingomyelinase); Gaucher's disease (glucocerebrosidase); Farber's disease (ceraminidase); Fabry's disease (α-galactosidase A); Krabbe's disease (galactosylceramide β-galactosidase); metachromatic leukodystrophy (arylsulfatase A); lysosomal acid lipase deficiency including Wolman's disease (lysosomal acid lipase); Batten disease (juvenile neuronal ceroid lipofuscinosis) (lysosomal trans-membrane CLN3 protein) sialidosis (neuraminidase 1); galactosialidosis (Goldberg's syndrome) (protective protein/cathepsin A); α-mannosidosis (α-D-mannosidase); β-mannosidosis (β-D-mannosidosis); fucosidosis (α-D-fucosidase); aspartylglucosaminuria (N-Aspartylglucosaminidase); and sialuria (Na phosphate cotransporter).

Exemplary glycogen storage diseases that can be treated according to the present invention include, but are not limited to, Type Ia GSD (von Gierke disease) (glucose-6-phosphatase), Type Ib GSD (glucose-6-phosphate translocase), Type Ic GSD (microsomal phosphate or pyrophosphate transporter), Type Id GSD (microsomal glucose transporter), Type II GSD including Pompe disease or infantile Type IIGSD (lysosomal acid α-glucosidase) and Type IIb (Danon) (lysosomal membrane protein-2), Type Ma and Mb GSD (Debrancher enzyme; amyloglucosidase and oligoglucanotransferase), Type IV GSD (Andersen's disease) (branching enzyme), Type V GSD (McArdle disease) (muscle phosphorylase), Type VI GSD (Hers' disease) (liver phosphorylase), Type VII GSD (Tarui's disease) (phosphofructokinase), GSD Type VIII/IXa (X-linked phosphorylase kinase), GSD Type IXb (Liver and muscle phosphorylase kinase), GSD Type IXc (liver phosphorylase kinase), GSD Type IXd (muscle phosphorylase kinase), GSD 0 (glycogen synthase), Fanconi-Bickel syndrome (glucose transporter-2), phosphoglucoisomerase deficiency, muscle phosphoglycerate kinase deficiency, phosphoglycerate mutase deficiency, fructose 1,6-diphosphatase deficiency, phosphoenolpyruvate carboxykinase deficiency, and lactate dehydrogenase deficiency.

Gene transfer has substantial potential use in understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using inhibitory RNA such as RNAi (e.g., siRNA or shRNA), microRNA or antisense RNA. For unbalanced disease states, gene transfer can be used to create a disease state in a model system, which can then be used in efforts to counteract the disease state. Thus, the virus vectors according to the present invention permit the treatment of genetic diseases. As used herein, a disease state is treated by partially or wholly remedying the deficiency or imbalance that causes the disease or makes it more severe. The use of site-specific recombination of nucleic sequences to cause mutations or to correct defects is also possible.

The virus vectors according to the present invention may also be employed to provide an antisense nucleic acid or inhibitory RNA (e.g., microRNA or RNAi such as a siRNA or shRNA) to a cell in vitro or in vivo. Expression of the inhibitory RNA in the target cell diminishes expression of a particular protein(s) by the cell. Accordingly, inhibitory RNA may be administered to decrease expression of a particular protein in a subject in need thereof. Inhibitory RNA may also be administered to cells in vitro to regulate cell physiology, e.g., to optimize cell or tissue culture systems.

As a further aspect, the virus vectors of the present invention may be used to produce an immune response in a subject. According to this embodiment, a virus vector comprising a nucleic acid encoding an immunogen may be administered to a subject, and an active immune response (optionally, a protective immune response) is mounted by the subject against the immunogen. Immunogens are as described hereinabove.

Alternatively, the virus vector may be administered to a cell ex vivo and the altered cell is administered to the subject. The heterologous nucleic acid is introduced into the cell, and the cell is administered to the subject, where the heterologous nucleic acid encoding the immunogen is optionally expressed and induces an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment of disease, in particular cancer or tumors (e.g., by causing regression of a cancer or tumor and/or by preventing metastasis and/or by preventing growth of metastatic nodules). The protective effects may be complete or partial, as long as the benefits of the treatment outweigh any disadvantages thereof.

The virus vectors of the present invention may also be administered for cancer immunotherapy by administration of a viral vector expressing a cancer cell antigen (or an immunologically similar molecule) or any other immunogen that produces an immune response against a cancer cell. To illustrate, an immune response may be produced against a cancer cell antigen in a subject by administering a viral vector comprising a heterologous nucleotide sequence encoding the cancer cell antigen, for example to treat a patient with cancer. The virus vector may be administered to a subject in vivo or by using ex vivo methods, as described herein.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to, leukemia, lymphoma (e.g., Hodgkin and non-Hodgkin lymphomas), colorectal cancer, renal cancer, liver cancer, breast cancer, lung cancer, prostate cancer, testicular cancer, ovarian cancer, uterine cancer, cervical cancer, brain cancer (e.g., gliomas and glioblastoma), bone cancer, sarcoma, melanoma, head and neck cancer, esophageal cancer, thyroid cancer, and the like. In embodiments of the invention, the invention is practiced to treat and/or prevent tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

Cancer cell antigens have been described hereinabove. By the terms “treating cancer” or “treatment of cancer,” it is intended that the severity of the cancer is reduced or the cancer is prevented or at least partially eliminated. For example, in particular contexts, these terms indicate that metastasis of the cancer is prevented or reduced or at least partially eliminated. In further representative embodiments these terms indicate that growth of metastatic nodules (e.g., after surgical removal of a primary tumor) is prevented or reduced or at least partially eliminated. By the terms “prevention of cancer” or “preventing cancer” it is intended that the methods at least partially eliminate or reduce the incidence or onset of cancer. Alternatively stated, the onset or progression of cancer in the subject may be slowed, controlled, decreased in likelihood or probability, or delayed.

In particular embodiments, cells may be removed from a subject with cancer and contacted with a virus vector according to the present invention. The modified cell is then administered to the subject, whereby an immune response against the cancer cell antigen is elicited. This method is particularly advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (e.g., CTL inductive cytokines) may be administered to a subject in conjunction with the virus vectors.

Cytokines may be administered by any method known in the art. Exogenous cytokines may be administered to the subject, or alternatively, a nucleotide sequence encoding a cytokine may be delivered to the subject using a suitable vector, and the cytokine produced in vivo.

The viral vectors are further useful for targeting liver cells for research purposes, e.g., for study of liver function in vitro or in animals or for use in creating and/or studying animal models of disease. For example, the vectors can be used to deliver heterologous nucleic acids to hepatocytes in animal models of liver injury, e.g., fibrosis or cirrhosis or animal models of liver diseases such as viral infections (e.g., hepatitis viruses).

Further, the virus vectors according to the present invention find further use in diagnostic and screening methods, whereby a gene of interest is transiently or stably expressed in a cell culture system, or alternatively, a transgenic animal model. The invention can also be practiced to deliver a nucleic acid for the purposes of protein production, e.g., for laboratory, industrial or commercial purposes.

The nucleic acid delivery vectors can also be used for various non-therapeutic purposes, including but not limited to use in protocols to assess gene targeting, clearance, transcription, translation, etc., as would be apparent to one skilled in the art. The nucleic acid delivery vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

### Pharmaceutical Formulations, Subjects, and Modes of Administration

Recombinant virus vectors according to the present invention (e.g., comprising an altered capsid protein as described herein) find use in both veterinary and medical applications. Suitable subjects include both avians and mammals. The term “avian” as used herein includes, but is not limited to, chickens, ducks, geese, quail, turkeys, pheasant, parrots, parakeets. The term “mammal” as used herein includes, but is not limited to, humans, primates non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a nucleic acid including those described herein. As a further option, the subject can be a laboratory animal and/or an animal model of disease.

In particular embodiments, the present invention provides a pharmaceutical composition comprising a virus vector comprising an altered capsid protein described herein in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and will preferably be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleotide sequence to a cell in vitro. The virus vector may be introduced to the cells at the appropriate multiplicity of infection according to standard transduction methods appropriate for the particular target cells. Titers of the virus vector or capsid to administer can vary, depending upon the target cell type and number, and the particular virus vector or capsid, and can be determined by those of skill in the art without undue experimentation. In particular embodiments, at least about 10³ infectious units, more preferably at least about 10⁵ infectious units are introduced to the cell.

The cell(s) into which the virus vector can be introduced may be of any type, including but not limited to neural cells (including cells of the peripheral and central nervous systems, in particular, brain cells such as neurons, oligodendrocytes, glial cells, astrocytes), lung cells, cells of the eye (including retinal cells, retinal pigment epithelium, and corneal cells), epithelial cells (e.g., gut and respiratory epithelial cells), skeletal muscle cells (including myoblasts, myotubes and myofibers), diaphragm muscle cells, dendritic cells, pancreatic cells (including islet cells), hepatic cells, a cell of the gastrointestinal tract (including smooth muscle cells, epithelial cells), heart cells (including cardiomyocytes), bone cells (e.g., bone marrow stem cells), hematopoietic stem cells, spleen cells, keratinocytes, fibroblasts, endothelial cells, prostate cells, joint cells (including, e.g., cartilage, meniscus, synovium and bone marrow), germ cells, and the like. Alternatively, the cell may be any progenitor cell. As a further alternative, the cell can be a stem cell (e.g., neural stem cell, liver stem cell). As still a further alternative, the cell may be a cancer or tumor cell (cancers and tumors are described above). Moreover, the cells can be from any species of origin, as indicated above.

The virus vectors may be introduced to cells in vitro for the purpose of administering the modified cell to a subject. In particular embodiments, the cells have been removed from a subject, the virus vector is introduced therein, and the cells are then replaced back into the subject. Methods of removing cells from subject for treatment ex vivo, followed by introduction back into the subject are known in the art (see, e.g., U.S. Pat. No. 5,399,346). Alternatively, the recombinant virus vector is introduced into cells from another subject, into cultured cells, or into cells from any other suitable source, and the cells are administered to a subject in need thereof.

Suitable cells for ex vivo gene therapy are as described above. Dosages of the cells to administer to a subject will vary upon the age, condition and species of the subject, the type of cell, the nucleic acid being expressed by the cell, the mode of administration, and the like. Typically, at least about 10² to about 10⁸ or about 10³ to about 10⁶ cells will be administered per dose in a pharmaceutically acceptable carrier. In particular embodiments, the cells transduced with the virus vector are administered to the subject in an effective amount in combination with a pharmaceutical carrier.

In some embodiments, cells that have been transduced with the virus vector may be administered to elicit an immunogenic response against the delivered polypeptide (e.g., expressed as a transgene or in the capsid). Typically, a quantity of cells expressing an effective amount of the polypeptide in combination with a pharmaceutically acceptable carrier is administered. Optionally, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of administering the virus vectors or capsids of the invention to subjects. In particular embodiments, the method comprises a method of delivering a nucleic acid of interest to an animal subject, the method comprising: administering an effective amount of a virus vector according to the invention to an animal subject. Administration of the virus vectors of the present invention to a human subject or an animal in need thereof can be by any means known in the art. Optionally, the virus vector is delivered in an effective dose in a pharmaceutically acceptable carrier.

The virus vectors of the invention can further be administered to a subject to elicit an immunogenic response (e.g., as a vaccine). Typically, vaccines of the present invention comprise an effective amount of virus in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the virus vectors to be administered to a subject will depend upon the mode of administration, the disease or condition to be treated, the individual subject's condition, the particular virus vector, and the nucleic acid to be delivered, and can be determined in a routine manner. Exemplary doses for achieving therapeutic effects are virus titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units or more, preferably about 10⁷ or 10⁸, 10⁹, 10¹⁰, 10¹¹, 10¹², 10¹³, 10¹⁴ or 10¹⁵ transducing units, yet more preferably about 10¹² to 10¹⁴ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or brain). Administration can also be to a tumor (e.g., in or a near a tumor or a lymph node). The most suitable route in any given case will depend on the nature and severity of the condition being treated and on the nature of the particular vector that is being used.

In some embodiments, the AAV particle is delivered directly to the CNS, e.g., by intrathecal, intracerebral, intraventricular, intranasal, intra-aural, intra-ocular, or peri-ocular delivery, or any combination thereof.

Administration can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The AAV particle can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the AAV particle is administered to a limb (arm and/or leg) of a subject (e.g., a subject with muscular dystrophy such as DMD) by limb perfusion, optionally isolated limb perfusion (e.g., by intravenous or intra-articular administration. In embodiments of the invention, the AAV particle can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the agent to cross the endothelial cell barrier. In particular embodiments, the AAV particle can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The AAV particle can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Administration to smooth muscle can be by any suitable method including intravenous administration, intra-arterial administration, and/or intra-peritoneal administration. In one embodiment, administration can be to endothelial cells present in, near, and/or on smooth muscle.

Delivery to a target tissue can also be achieved by delivering a depot comprising the AAV particle. In representative embodiments, a depot comprising the AAV particle is implanted into skeletal, smooth, cardiac and/or diaphragm muscle tissue or the tissue can be contacted with a film or other matrix comprising the AAV particle. Such implantable matrices or substrates are described in U.S. Pat. No. 7,201,898.

Administration can also be to a tumor (e.g., in or near a tumor or a lymph node). The most suitable route in any given case will depend on the nature and severity of the condition being treated and/or prevented and on the nature of the particular vector that is being used.

The AAV particle may be delivered or targeted to any tissue or organ in the subject. In some embodiments, the heterologous agent and the cell membrane fusion protein or a derivative thereof are administered to, e.g., a skeletal muscle, a smooth muscle, the heart, the diaphragm, the airway epithelium, the liver, the kidney, the spleen, the pancreas, the skin, the lung, the ear, and the eye. In some embodiments, the heterologous agent and the cell membrane fusion protein or a functional fragment or derivative thereof is administered to a diseased tissue or organ, e.g., a tumor.

Typically, the viral vector will be administered in a liquid formulation by systemic delivery or direct injection to the desired region or compartment. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. Controlled release of parvovirus and AAV vectors is described by international patent publication WO 01/91803.

Delivery to any of these tissues can also be achieved by delivering a depot comprising the virus vector, which can be implanted into the tissue or the tissue can be contacted with a film or other matrix comprising the virus vector. Examples of such implantable matrices or substrates are described in U.S. Pat. No. 7,201,898).

[Injectables can be prepared in conventional forms, either as liquid solutions or suspensions, solid forms suitable for solution or suspension in liquid prior to injection, or as emulsions. Alternatively, one may administer the virus vector in a local rather than systemic manner, for example, in a depot or sustained-release formulation. Further, the virus vector can be delivered dried to a surgically implantable matrix such as a bone graft substitute, a suture, a stent, and the like (e.g., as described in U.S. Pat. No. 7,201,898).

Pharmaceutical compositions suitable for oral administration can be presented in discrete units, such as capsules, cachets, lozenges, or tablets, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a virus vector of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions suitable for parenteral administration can comprise sterile aqueous and non-aqueous injection solutions of the composition of this invention, which preparations are optionally isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration can be presented as unit dose suppositories. These can be prepared by admixing the composition with one or more conventional solid carriers, such as for example, cocoa butter and then shaping the resulting mixture.

Pharmaceutical compositions of this invention suitable for topical application to the skin can take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharm. Res. 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

The virus vectors disclosed herein may be administered to the lungs of a subject by any suitable means, for example, by administering an aerosol suspension of respirable particles comprised of the virus vectors, which the subject inhales. The respirable particles may be liquid or solid. Aerosols of liquid particles comprising the virus vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors may likewise be produced with any solid particulate medicament aerosol generator, by techniques known in the pharmaceutical art.

Having described the present invention, the same will be explained in greater detail in the following examples, which are included herein for illustration purposes only, and which are not intended to be limiting to the invention.

The present invention may be as defined in any one of the following numbered paragraphs.

1. An adeno-associated virus (AAV) capsid protein or derivative thereof, comprising at least a portion of an AAV VP1/VP2 boundary, the capsid protein comprising an amino acid sequence modification at one or more amino acids within the VP1/VP2 boundary that alters permissiveness of a promoter within a cell when the promoter and the capsid protein are present within the cell, and wherein the capsid protein and the promoter are in the context of a recombinant AAV particle.
2. The AAV capsid protein or derivative thereof, of paragraph 1, wherein the VP1/VP2 boundary corresponds to amino acid 110-170 of AAV9 VP1.
3. The AAV capsid protein or derivative thereof of paragraph 1 or 2, wherein the amino acid sequence modification alters the electrostatic charge of the VP1/VP2 boundary of the capsid that forms the particle thereby altering the permissiveness of the promoter within the cell.
4. The AAV capsid protein or derivative thereof of any of paragraphs 1-3, wherein the amino acid sequence modification comprises a mutation selected from the group consisting of an insertion, a deletion, a substitution, and combinations thereof.
5. The AAV capsid protein or derivative thereof of any one of paragraphs 1-4, wherein the amino acid sequence modification comprises a modification at one or more amino acids corresponding to from 120 to 150 of AAV9 VP1.
6. The AAV capsid protein or derivative thereof of any one of paragraphs 1-5, wherein the cell is a neuronal cell or a glial cell.
7. The AAV capsid protein or derivative thereof of paragraph 6, wherein the neuronal cell is a medium spiny neuron, a cholinergic interneuron, or a GABAergic interneuron.
8. The AAV capsid protein or derivative thereof of paragraph 6, wherein the glial cell is an oligodendrocyte, microglia, or astrocyte.
9. The AAV capsid protein or derivative thereof of any one of paragraphs 1-8, wherein the promoter is a ubiquitous promoter.
10. The AAV capsid protein or derivative thereof of any one of paragraphs 1-8, wherein the promoter is a tissue specific promoter.
11. The AAV capsid protein or derivative thereof of any one of paragraphs 1-11, wherein the amino acid sequence modification is an insertion of from 1-20 amino acid residues, from 4-16 amino acid residues, from 6-14 amino acid residues, or from 3-200 amino acid residues.
12. The AAV capsid protein or derivative thereof of any one of paragraphs 1-11, wherein the amino acid sequence modification is an insertion of 3 or more amino acid residues.
13. The AAV capsid protein or derivative thereof of any one of paragraphs 1-12, wherein the amino acid sequence modification preserves nuclear localization signals and phospholipase domains present in the capsid protein.
14. The AAV capsid protein or derivative thereof of any one of paragraphs 1-13, wherein the amino acid sequence modification is a modification to the amino acid sequence of VP2.
15. The AAV capsid protein or derivative thereof of any one of paragraphs 1-14, wherein the amino acid sequence modification is an insertion between amino acids corresponding to 137 and 138 of AAV9 capsid protein, or between amino acids corresponding to 138 and 139 of AAV9 capsid protein.
16. The AAV capsid protein or derivative thereof of any one of paragraphs 1-15, wherein the amino acid sequence modification increases expression from the promoter in oligodendrocytes and/or decreases expression from the promoter in neurons.
17. The AAV capsid protein or derivative thereof of any one of paragraphs 1-16, wherein the amino acid sequence modification is an insertion of residues having an overall negative charge.
18. The AAV capsid protein or derivative thereof of paragraph 17, wherein the amino acid sequence modification is an insertion of 2 or more glutamate residues.
19. The AAV capsid protein or derivative thereof of paragraph 18, wherein the amino acid sequence modification is an insertion of 6 glutamate residues.
20. The AAV capsid protein or derivative thereof of any one of paragraphs 1-16, wherein the amino acid sequence modification is an insertion of residues having an overall positive charge.
21. The AAV capsid protein or derivative thereof of paragraph 20, wherein the amino acid sequence modification is an insertion of substance P peptide.
22. The AAV capsid protein or derivative thereof of any one of any one of paragraphs 1-15, wherein the amino acid sequence modification increases expression from the promoter in neurons and/or decreases expression from the promoter in oligodendrocytes.
23. The AAV capsid protein or derivative thereof of any one of paragraphs 1-22, wherein the amino acid sequence modification is an insertion of residues having an overall neutral charge.
24. The AAV capsid protein or derivative thereof of paragraph 23, wherein the amino acid sequence modification is an insertion of 2 or more alanine residues.
25. The AAV capsid protein or derivative thereof of paragraph 24, wherein the amino acid sequence modification is an insertion of 6 alanine residues.
26. The AAV capsid protein or derivative thereof of any of paragraphs 1-25, wherein the capsid protein is from a serotype selected from the group consisting of AAV1, AAV2, AAV3, AAV4, AAV5, AAV6, AAV7, AAV8, AAV9, AAV10, AAV11, AAV12, and AAV13.
27. The AAV capsid protein or derivative thereof of paragraph 26, wherein the capsid protein is from AAV2.
28. The AAV capsid protein or derivative thereof of paragraph 26, wherein the capsid protein is from AAV8.
29. The AAV capsid protein or derivative thereof of paragraph 26, wherein the capsid protein is from AAV9.
30. The AAV capsid protein or derivative thereof of any of paragraphs 1-26, wherein the rAAV particle is selected from the group consisting of AAV1, AAV2, AAV3, AAV4, AAV5, AAV6, AAV7, AAV8, AAV9, AAV10, AAV11, AAV12, and AAV13.
31. The AAV capsid protein or derivative thereof of paragraph 30, wherein the rAAV particle is AAV2.
32. The AAV capsid protein or derivative thereof of paragraph 30, wherein the rAAV particle is AAV8.
33. The AAV capsid protein or derivative thereof of paragraph 30, wherein the rAAV particle is AAV9.
34. A method for altering expression of a transgene operably linked to a promoter and delivered to a cell by a rAAV vector comprising modifying the amino acid sequence of at least one amino acid within the VP1/VP2 boundary of a capsid protein or derivative thereof of the rAAV vector, wherein the amino acid sequence modification alters the permissiveness of the promoter within the cell.
35. The method of paragraph 34, wherein the VP1/VP2 boundary corresponds to amino acid 110-170 of AAV9.
36. The method of paragraph 34 or 35, wherein the amino acid sequence modification alters the electrostatic charge of the VP1/VP2 boundary of the capsid that forms the particle thereby altering the permissiveness of the promoter within the cell.
37. The method of any one of paragraphs 34-36, wherein the amino acid sequence modification comprises a mutation selected from the group consisting of an insertion, a deletion, a substitution, and combinations thereof.
38. The method of any one of paragraphs 34-37, wherein the amino acid sequence modification comprises a modification at one or more amino acids corresponding to from 120 to 150 of AAV9 VP1.
39. The method of any one of paragraphs 34-38, wherein the cell is a neuronal cell or a glial cell.
40. The method of paragraph 39, wherein the neuronal cell is a medium spiny neuron, a cholinergic interneuron, or a GABAergic interneuron.
41. The method of paragraph 39, wherein the glial cell is an oligodendrocyte, microglia, or astrocyte.
42. The method of any one of paragraphs 34-41, wherein the promoter is a ubiquitous promoter.
43. The method of any one of paragraphs 34-41, wherein the promoter is a tissue specific promoter.
44. The method of any one of paragraphs 34-41, wherein the promoter is the CBA promoter.
45. The method of any one of paragraphs 34-41, wherein the promoter is the CBh promoter.
46. The method of any one of paragraphs 34-41, wherein the promoter is the JeTI promoter.
47. The method of any one of paragraphs 34-41, wherein the promoter is the synapsin promoter.
48. The method of any one of paragraphs 34-41, wherein the promoter is cytomegalovirus (CMV) immediate-early enhancer and chicken beta-actin (CAG), cytomegalovirus (CMV), CMV/chicken β-actin (CMV/(β-actin), elongation factor 1α (EF1α), phosphoglycerate kinase, ubiquitin C (UbC), CB, CBA, and CBh, JeTI, human synapsin promoter (hSYN1), cytomegalovirus (CMV) promoter, or a CB7 promoter, the human β-actin promoter, the human elongation factor-1 α promoter, the simian virus 40 promoter, and the herpes simplex virus thymidine kinase promoter, dihydrofolate reductase promoter, the phosphoglycerol kinase (PGK) promoter, the rhodopsin kinase promoter, the rhodopsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter, the ornithine transcarbamylase (OTC) promoter the alpha 1-antitrypsin (AAT) promoter, the albumin promoter, hepatitis B virus core promoter, thyroxin binding globulin (TGB) promoter and the LSP1 promoter, skeletal β-actin promoter, myosin light chain 2A, dystrophin, muscle creatine kinase, liver (albumin) promoter, hepatitis B virus core promoter, alpha-fetoprotein (AFP), bone osteocalcin, bone sialoprotein, lymphocytes (CD2), immunoglobulin heavy chain, T cell receptor chain, neuron-specific enolase (NSE) promoter, neurofilament light-chain gene promoter, the neuron-specific vgf gene promoter, Synapsinl, CaMKII alpha, GAD67, GAD65, VGAT, SP-B promoter, ICAM promoter, IFN beta promoter, CD45 promoter, OG-2 promoter, NSE promoter, MSK promoter, JeTI, SPc5-12, 2R5Sc5-12, dMCK, tMCK, SP1 element, synthetic cold-shock responsive promoter (calcyclin), sps5, sps8, synthetic promoter inducible under acidic conditions derived from the YGP1 gene, synthetic promoter inducible under acidic conditions derived from the CCW14 gene, YGP1pr, tTA, tTA-dependent promoter, cumate inducible promoter, 4-hydroxytamoxifen (OHT)-inducible promoter, metallothionein-inducible promoter, rapamycin-inducible promoter, or Rheoswitch® inducible promoter.
49. The method of any one of paragraphs 34-48, wherein the amino acid sequence modification is an insertion of from 1-20 amino acid residues, from 4-16 amino acid residues, from 6-14 amino acid residues, or from 3-200 amino acid residues.
50. The method of any one of paragraphs 34-49, wherein the amino acid sequence modification is an insertion of 3 or more amino acid residues.
51. The method of any one of paragraphs 34-50, wherein the amino acid sequence modification preserves nuclear localization signals and phospholipase domains present in the capsid protein.
52. The method of any one of paragraphs 34-51, wherein the amino acid sequence modification is a modification to the amino acid sequence of VP2.
53. The method of any one of paragraphs 34-52, wherein the amino acid sequence modification is an insertion between amino acids corresponding to 137 and 138 of AAV9, or between amino acids corresponding to 138 and 139 of AAV9.
54. The method of any one of paragraphs 34-53, wherein the amino acid sequence modification increases expression from the promoter in oligodendrocytes and/or decreases expression from the promoter in neurons.
55. The method of any one of paragraphs 34-54, wherein the amino acid sequence modification is an insertion of residues having an overall negative charge.
56. The method of paragraph 55, wherein the amino acid sequence modification is an insertion of 2 or more glutamate residues.
57. The method of paragraph 56, wherein the amino acid sequence modification is an insertion of 6 glutamate residues.
58. The method of any one of paragraphs 34-54, wherein the amino acid sequence modification is an insertion of residues having an overall positive charge.
59. The method of paragraph 34-53, wherein the amino acid sequence modification is an insertion of substance P peptide.
60. The method of any one of any one of paragraphs 34-53, wherein the amino acid sequence modification increases expression from the promoter in neurons and/or decreases expression of from the promoter in oligodendrocytes.
61. The method of any one of paragraphs 34-53, wherein the amino acid sequence modification is an insertion of residues having an overall neutral charge.
62. The method of paragraph 61, wherein the amino acid sequence modification is an insertion of 2 or more alanine residues.
63. The method of paragraph 62, wherein the amino acid sequence modification is an insertion of 6 alanine residues.
64. The method of any of paragraphs 34-63, wherein the capsid protein is from a serotype selected from the group consisting of AAV1, AAV2, AAV3, AAV4, AAV5, AAV6, AAV7, AAV8, AAV9, AAV10, AAV11, AAV12, and AAV13.
65. The AAV capsid protein or derivative thereof of paragraph 64, wherein the capsid protein is from AAV2.
66. The AAV capsid protein or derivative thereof of paragraph 64, wherein the capsid protein is from AAV8.
67. The AAV capsid protein or derivative thereof of paragraph 64, wherein the capsid protein is from AAV9.
68. The method of any of paragraphs 34-67, wherein the rAAV particle is selected from the group consisting of AAV1, AAV2, AAV3, AAV4, AAV5, AAV6, AAV7, AAV8, AAV9, AAV10, AAV11, AAV12, and AAV13.
69. The AAV capsid protein or derivative thereof of paragraph 68, wherein the rAAV particle is AAV2.
70. The AAV capsid protein or derivative thereof of paragraph 568, wherein the rAAV particle is AAV8.
71. The AAV capsid protein or derivative thereof of paragraph 68, wherein the rAAV particle is AAV9.
72. A nucleic acid encoding the AAV capsid protein or derivative thereof of any one of paragraphs 1-33.
73. The nucleic acid of paragraph 72, wherein the nucleic acid is comprised within a vector.
74. The nucleic acid of paragraph 73, wherein the vector is a plasmid, phage, viral vector, bacterial artificial chromosome, or yeast artificial chromosome.
75. The nucleic acid of paragraph 74, wherein the viral vector is an AAV vector.
76. The nucleic acid of paragraph 75, wherein the nucleic acid further comprises an AAV rep coding sequence.
77. The nucleic acid of any one of paragraphs 73-76, wherein the vector further comprises a promoter operably linked to a heterologous polynucleotide.
78. The nucleic acid of paragraph 77, wherein the promoter is a synthetic promoter.
79. The nucleic acid of paragraph 77, wherein the promoter is the CBA promoter.
80. The nucleic acid of paragraph 77, wherein the promoter is the CBh promoter.
81. The nucleic acid of paragraph 77, wherein the promoter is the JeTI promoter.
82. The nucleic acid of paragraph 77, wherein the promoter is the synapsin promoter.
83. The nucleic acid of paragraph 77, wherein the promoter is cytomegalovirus (CMV) immediate-early enhancer and chicken beta-actin (CAG), cytomegalovirus (CMV), CMV/chicken β-actin (CMV/β-actin), elongation factor 1α (EF1α), phosphoglycerate kinase, ubiquitin C (UbC), CB, CBA, and CBh, JeTI, human synapsin promoter (hSYN1), cytomegalovirus (CMV) promoter, or a CB7 promoter, the human β-actin promoter, the human elongation factor-1 α promoter, the simian virus 40 promoter, and the herpes simplex virus thymidine kinase promoter, dihydrofolate reductase promoter, the phosphoglycerol kinase (PGK) promoter, the rhodopsin kinase promoter, the rhodopsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter, the ornithine transcarbamylase (OTC) promoter the alpha 1-antitrypsin (AAT) promoter, the albumin promoter, hepatitis B virus core promoter, thyroxin binding globulin (TGB) promoter and the LSP1 promoter, skeletal β-actin promoter, myosin light chain 2A, dystrophin, muscle creatine kinase, liver (albumin) promoter, hepatitis B virus core promoter, alpha-fetoprotein (AFP), bone osteocalcin, bone sialoprotein, lymphocytes (CD2), immunoglobulin heavy chain, T cell receptor chain, neuron-specific enolase (NSE) promoter, neurofilament light-chain gene promoter, the neuron-specific vgf gene promoter, Synapsinl, CaMKII alpha, GAD67, GAD65, VGAT, SP-B promoter, ICAM promoter, IFN beta promoter, CD45 promoter, OG-2 promoter, NSE promoter, MSK promoter, JeTI, SPc5-12, 2R5Sc5-12, dMCK, tMCK, SP1 element, synthetic cold-shock responsive promoter (calcyclin), sps5, sps8, synthetic promoter inducible under acidic conditions derived from the YGP1 gene, synthetic promoter inducible under acidic conditions derived from the CCW14 gene, YGP1pr, tTA, tTA-dependent promoter, cumate inducible promoter, 4-hydroxytamoxifen (OHT)-inducible promoter, metallothionein-inducible promoter, rapamycin-inducible promoter, or Rheoswitch® inducible promoter.
84. A cell in vitro comprising the nucleic acid of any one of paragraphs 72-83 stably incorporated into the genome.
85. A virus particle comprising the nucleic acid of any one of paragraphs 72-83.
86. The virus particle of paragraph 85, wherein the virus particle is an AAV particle, an adenovirus particle, a herpesvirus particle, or a baculovirus particle.
87. An AAV particle comprising:

an AAV vector genome; and

the AAV capsid protein or derivative thereof of any one of paragraphs 1-33, wherein the AAV capsid protein or derivative thereof encapsidates the AAV vector genome.

88. The AAV particle of paragraph 87, wherein the AAV vector genome comprises a promoter operably linked to a heterologous nucleic acid.
89. The AAV particle of paragraph 88, wherein the promoter is a synthetic promoter.
90. The AAV particle of paragraph 88, wherein the promoter is the CBA promoter.
91. The AAV particle of paragraph 88, wherein the promoter is the CBh promoter.
92. The AAV particle of paragraph 88, wherein the promoter is the JeTI promoter.
93. The AAV particle of paragraph 88, wherein the promoter is the synapsin promoter.
94. The AAV particle of paragraph 88, wherein the promoter is cytomegalovirus (CMV) immediate-early enhancer and chicken beta-actin (CAG), cytomegalovirus (CMV), CMV/chicken β-actin (CMV/β-actin), elongation factor 1α (EF1α), phosphoglycerate kinase, ubiquitin C (UbC), CB, CBA, and CBh, JeTI, human synapsin promoter (hSYN1), cytomegalovirus (CMV) promoter, or a CB7 promoter, the human β-actin promoter, the human elongation factor-1 α promoter, the simian virus 40 promoter, and the herpes simplex virus thymidine kinase promoter, dihydrofolate reductase promoter, the phosphoglycerol kinase (PGK) promoter, the rhodopsin kinase promoter, the rhodopsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter, the ornithine transcarbamylase (OTC) promoter the alpha 1-antitrypsin (AAT) promoter, the albumin promoter, hepatitis B virus core promoter, thyroxin binding globulin (TGB) promoter and the LSP1 promoter, skeletal β-actin promoter, myosin light chain 2A, dystrophin, muscle creatine kinase, liver (albumin) promoter, hepatitis B virus core promoter, alpha-fetoprotein (AFP), bone osteocalcin, bone sialoprotein, lymphocytes (CD2), immunoglobulin heavy chain, T cell receptor chain, neuron-specific enolase (NSE) promoter, neurofilament light-chain gene promoter, the neuron-specific vgf gene promoter, Synapsinl, CaMKII alpha, GAD67, GAD65, VGAT, SP-B promoter, ICAM promoter, IFN beta promoter, CD45 promoter, OG-2 promoter, NSE promoter, MSK promoter, JeTI, SPc5-12, 2R5Sc5-12, dMCK, tMCK, SP1 element, synthetic cold-shock responsive promoter (calcyclin), sps5, sps8, synthetic promoter inducible under acidic conditions derived from the YGP1 gene, synthetic promoter inducible under acidic conditions derived from the CCW14 gene, YGP1pr, tTA, tTA-dependent promoter, cumate inducible promoter, 4-hydroxytamoxifen (OHT)-inducible promoter, metallothionein-inducible promoter, rapamycin-inducible promoter, or Rheoswitch® inducible promoter.
95. The AAV particle of any one of paragraphs 88-94, wherein the heterologous nucleic acid encodes an antisense RNA, microRNA, or RNAi.
96. The AAV particle of any one of paragraphs 88-94, wherein the heterologous nucleic acid encodes a polypeptide.
97. The AAV particle of paragraph 96, wherein the heterologous nucleic acid encodes a therapeutic polypeptide.
98. The AAV particle of paragraph 96, wherein the heterologous nucleic acid encodes a reporter protein.
99. A method of producing a recombinant AAV particle comprising an AAV capsid, the method comprising:

providing a cell in vitro with a nucleic acid according to any one of paragraphs 72-83, an AAV rep coding sequence, an AAV vector genome comprising a promoter operably linked to a heterologous nucleic acid, and helper functions for generating a productive AAV infection; and

allowing assembly of the recombinant AAV particle comprising the AAV capsid and encapsidating the AAV vector genome.

100. An AAV particle produced by the method of paragraph 99.
101. A pharmaceutical formulation comprising the AAV capsid protein or derivative thereof of any one of paragraphs 1-33, the nucleic acid of any one of paragraphs 72-83, the virus particle of paragraph 85 or 86, or the AAV particle of any one of paragraphs 87-98 or 100 in a pharmaceutically acceptable carrier.
102. A method of delivering a nucleic acid of interest to a cell, the method comprising contacting the cell with the AAV particle of any one of paragraphs 87-98 or 100.
103. The method of paragraph 102, wherein the cell is a central nervous system (CNS) cell.
104. A method of delivering a nucleic acid of interest to a cell in a mammalian subject, the method comprising:

administering an effective amount of the AAV particle of any one of paragraphs 87-98 or 100 or the pharmaceutical formulation of paragraph 101 to a mammalian subject, thereby delivering the nucleic acid of interest to a cell in the mammalian subject.

105. The method of paragraph 104, wherein the cell is a central nervous system cell.
106. The method of paragraph 104 or 105, wherein the mammalian subject is a human subject.
107. The method of any one of paragraphs 104-106, wherein the AAV particle is delivered to the CNS.
108. The method of paragraph 107, wherein the AAV particle is delivered directly to the CNS by intrathecal, intracerebral, intraventricular, intranasal, intra-aural, intra-ocular, or peri-ocular delivery, or any combination thereof.
109. A method of treating a disorder in a mammalian subject in need thereof, wherein the disorder is treatable by expressing a therapeutic product in cells of the subject, the method comprising administering a therapeutically effective amount of the AAV particle of any one of paragraphs 87-98 or 100 or the pharmaceutical formulation of paragraph 80 to a mammalian subject, wherein the product is expressed, thereby treating the disorder.
110. The method of paragraph 109, wherein the cells of the subject are in the CNS.
111. A method of altering expression of a heterologous polynucleotide present in an AAV vector in cells of a subject, comprising preparing the AAV vector with the AAV capsid protein or derivative thereof of any one of paragraphs 1-33.
112. The method of paragraph 111, where the cells are CNS cells.

### EXAMPLES

### Example 1—AAV Capsid-Promoter Interactions Determine CNS Cell Selective Gene Expression in Vivo

### Methods:

Cloning construct: In order to directly compare the transgenes promoters were constructed by inserting a DNA stuffer into pAAV-CMV-mCherry-hGHpolyA using SacII. The CBA promoter was digested out of an existing construct using BglII (blunted) and Sal1 then ligated into pAAV-mCherry-DNAstuffer-hGHpoly at Mlu1 (blunted) and Sal1 sites. The CBh promoter was digested out of an existing construct using Kpn1 (blunted) and Age1 then ligated into pAAV-mCherry-DNAstuffer-hGHpoly at Mlu1 (blunted) and Age1 sites.

Virus Production: The virus was produced in HEK293 cells as previously described (Deverman et al., Nat. Biotechnol. 34:204 (2016)). Briefly, polyethylenimine max (PEI) was used for the triple transfection of a cap and rep plasmid (pGSK2/9 (AAV9), pGSK2/9EU, pSGK2/9AU, pXR2 (AAV2), pXR2AU and pXR2EU), the pXX6-80 helper plasmid, and a transgene plasmid (pAAV-CBA-mCherry-DNAstuffer-hGHpolyA, pAAV-CBh-mCherryDNA-stuffer-hGHpolyA, pTR-MBP-GFP, pTR-CBh-GFP). Cells were harvested 48 hr post-transfection, and the virus was purified by cesium chloride ultracentrifugation. After identifying peak fractions by qPCR, the virus was dialyzed into PBS/NaCl/D-Sorbitol. Titers were calculated by qPCR according to established procedures using a LightCycler 480 instrument and ITR primers. The individual titers were AAV2-CBA-1.9×10¹² vg/ml; AAV2-CBh-1.3×10¹³ vg/ml; AAV9-CBA-6×10¹³ vg/ml; AAV9-CBh-2.5×10¹³ vg/ml; AAV2EU-CBA-5×10¹² vg/ml; AAV2AU-CBA-2×10¹² vg/ml; AAV9AU-CBh-3.6×10¹² vg/ml; scAAV9-MBP-3×10¹² vg/ml; scAAV9AU-MBP-1.5×10¹² vg/ml; scAAV9-CBh-GFP-1.4×10¹² vg/ml.

Animals and Stereotactic Infusions: All of the animals were male Sprague-Dawley rats (Charles River Laboratories) weighing between 200 and 300 grams at the time of intracranial injection. The animals were maintained on a 12-hr light-dark cycle and had free access to water and food. For all animal studies, care and procedures were in accordance with the NIH Guide for the Care and Use of Laboratory Animals, and all procedures received prior approval by the University of North Carolina Institutional Animal Care and Usage Committee.

[Virus vector infusions were performed as previously described (Powell et al., Gene Ther. 23:807 (2016); Gray et al., Human Gene Ther. 22:1143 (2011)). First, animals were anesthetized with 50 mg/kg pentobarbital and placed into a stereotactic frame. Using a 32G stainless steel injector and a Sage infusion pump, animals received 2 μl unilaterally of each vector over 10 min into the striatum (0.5 mm anterior to bregma, 3.5 mm lateral, and 5.5 mm vertical, according to the atlas of Paxinos and Watson (Ojala et al., Mol. Ther. 26:305 (2018)). The injector was left in place for 3 min post-infusion in order to allow diffusion from the injector. The animal numbers were as follows: AAV2-CBA-mCherry (N=4); AAV2-CBh-mCherry (N=6); AAV9-CBA-mCherry (N=6); AAV9-CBh-mCherry (N=6); AAV2EU-CBA-mCherry (N=2); AAV9EU-CBA-mCherry (N=5); AAV9-MBP-GFP (N=2); AAV9-CBh-GFP (N=2).

Immunochemistry and Confocal Microscopy: Two weeks after AAV vector infusion, animals received an overdose of pentobarbital (100 mg/kg pentobarbital i.p.), and they were perfused transcardially with ice-cold 100 mM PBS (pH 7.4), followed by 4% paraformaldehyde in PB (pH 7.4). After brains were post-fixed 12-48 hr at 4° C. in the paraformaldehyde-PB, 40-μm coronal sections were cut using a vibrating blade microtome for subsequent immunofluorescence. To determine fluorescent transgene (mCherry or GFP) cellular co-localization, tissue sections were incubated in the blocking solution with either cellular markers antibodies: NeuN (1:500, Chemicon) or Olig2 (1:250, Abcam). Following incubation at 4° C. for 48-72 hr in primary antibodies, the sections were rinsed three times with PBS and blocked again for 45 min at room temperature. Subsequently, the tissue sections were incubated in either Alexafluor 488 or 594-conjugated goat anti-rabbit IgG or goat anti-mouse (1:500, Invitrogen) for 1 hr at 4° C. Rinsed sections were mounted, and fluorescence was visualized using an Olympus FV3000RS confocal microscope in the UNC Neuroscience Center Confocal. Transgene fluorescence co-localization was determined on the z stacks and counted using ImageJ Cellcounter plug in. At least 125 individual cells were counted per vector and antibody. The cell counts were tallied and basic statistics were performed in Excel. Cell counts are reported as percent co-localization (±SEM). R (R Studio Team (2015) R Studio: Integrated development for R. RStudio Inc, Boston, Mass.) was used to graph and perform independent t tests to test for significance.

### Results:

### In the Context of AAV9 Striatal Cell Transgene Expression is Promoter Dependent

Previously, we have found that AAV2 vector gene expression was restricted to striatal neurons, regardless of whether gene expression was driven by a full-length CBA promoter or the truncated constitutive CBh promoter that we created for self-complimentary AAV vectors (Gray et al., Human Gene Ther. 22:1143 (2011)). However, preliminary studies with AAV9 suggested in vivo differences in cellular gene expression patterns between the two promoters. Thus, in order to make a valid comparison, we constructed identical reporter gene cassettes where mCherry gene expression was driven by either the CBA (1.6 kb) or CBh (0.8 kb) promoter (FIG. 1A), but contained an identical length and sequence DNA stuffer, an identical Poly A, and identical ITRs (FIG. 1B). All vectors were directly infused into wild type rat striata (FIG. 2) at comparable titers and volumes. Two weeks post-vector infusion, the identity of transduced cell types was determined by co-localization with known cellular specific markers using immunofluorescence. AAV2-CBA-mCherry and AAV2-CBh-mCherry vectors exhibited a dominant neuronal gene expression regardless of the promoter (FIGS. 3A-3R). mCherry positive cells co-localized with the neuronal marker, NeuN (FIGS. 3A-3D and 3J-3M) in 87.5%±1.7 of cells and 80.4%±3.2 of cells when gene expression was driven by CBA or CBh, respectively (FIGS. 3I and 3R). Conversely, only 7.3%±1.3 and 9.7%±4.1 of mCherry positive cells co-localized with an oligodendrocyte marker (Olig2) (FIGS. 3E-3H and 3N-3Q), when gene expression was driven by a CBA or CBh promoter, respectively (FIGS. 3I and 3R). Although for AAV2, both promoters exhibit a preferential neuronal gene expression, it should be noted that in the context of single stranded AAV vectors, the CBh promoter does not drive gene expression to the same extent as the full length CBA promoter.

Surprisingly, the CBA and CBh promoters exhibited divergent cellular gene expression patterns in otherwise identical AAV9 vectors. When AAV9-CBA-mCherry vectors were infused into the rat striatum, 88.4%±1.6 of the mCherry positive cells co-localized with NeuN (FIGS. 3S-3V and 3A′), and only 10.4%±2.1 of mCherry positive cells co-localized with Olig2 (FIGS. 3W-3Z and 3A′). In marked contrast, AAV9-CBh-mCherry transgene expression co-localized with NeuN (FIGS. 3B′-3E′) in 46.3%±4.6 of the mCherry+ cells (FIG. 3J′), while 37.8%±4.9 of the remaining mCherry positive cells co-localized with Olig2 (FIGS. 3F′-3I′ and 3J′). Given that in both instances the capsid was identical, the AAV9 vectors inevitably gained identical cellular access and were trafficked to the nucleus of neurons and oligodendrocytes in the rat striatum. Also, with regard to the promoter activity with AAV2, the CBh promoter supports gene expression in striatal neurons (FIGS. 3J-3M), while in the context of an oligodendrocyte preferring AAV capsid, previous studies have shown that the CBA promoter supports gene expression in striatal oligodendrocytes (Powell et al., Gene Ther. 23:807 (2016); Weinberg et al., Mol. Ther. 25:928 (2017)). Because both promoters can support gene expression in striatal neurons and oligodendrocytes, the divergence in cellular gene expression patterns suggests that the AAV9 capsid exerted a significant influence on promoter activity across different CNS cell types.

### AAV9-CBA Striatal Transgene Expression is Shifted to Oligodendrocytes by a Six Glutamate Insertion in VP1/2

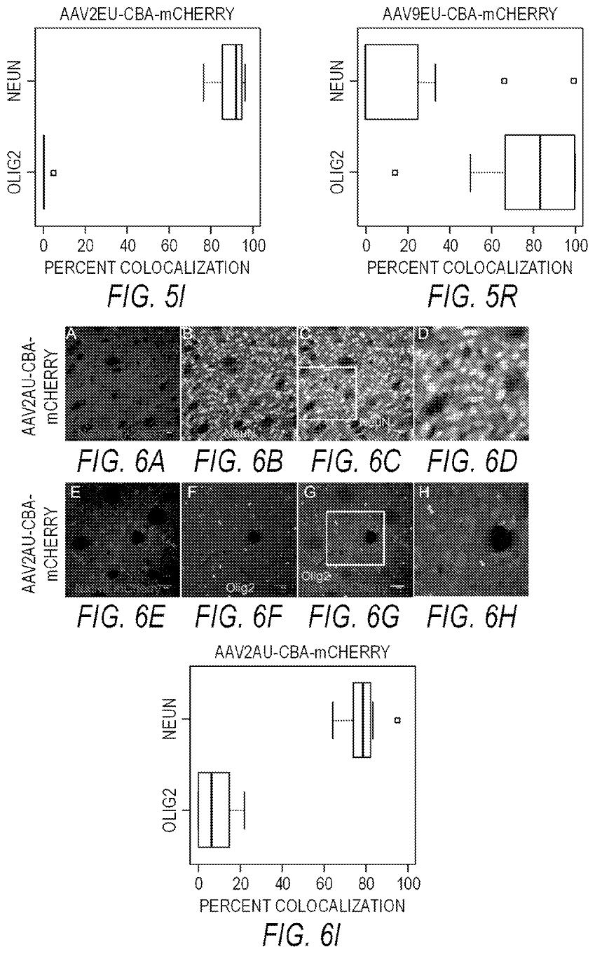

Mutations in VP2 capsids can alter gene expression in vitro (Johnson et al., J. Virol. 84:8888 (2010)), so we sought to disrupt a positively charged/basic region of VP1/2 intersection within the AAV capsid by inserting six glutamates after amino acid 138 into AAV2 (AAV2EU) and AAV9 (AAV9EU) (FIG. 4). The mutant capsids were packaged with the CBA promoter construct, directly infused into rat striatum and cellular expression was determined. The insertion of six glutamates into AAV2 (AAV2EU-CBA-mCherry) did not alter the cellular transgene expression from that of AAV2 shown in FIGS. 3A-3J′. In the striatum the mCherry positive cells predominately co-localized with NeuN (89.7%±2.7) and rarely co-localized with Olig2 (0.83%±0.8)(FIGS. 5A-5I). However, AAV9EU-CBA-mCherry exhibited little co-localization with NeuN (14.2%±3.6) (FIGS. 5J-5M and 5R), a marked departure to findings with AAV9-CBA (FIGS. 3S-3V and 3A′). This reduction of mCherry positive neurons in AAV9EU-CBA was significantly lower compared to AAV9-CBA (p<0.001). In marked contrast, AAV9EU-CBA mCherry expression co-localized with Olig2 (79.9%±4.6) (FIGS. 5N-5R). These results suggest that the AAV9EU capsid exerted a significant influence on in vivo cellular gene expression in a manner that previously has not been described.

AAV9 Striatal Transgene Expression is Neuronal after a Six Alanine Insertion into VP1/2

Next, to assess if the insertion itself was responsible for the change in cellular expression, six neutral alanine residues were inserted into the same VP1/VP2 site in AAV2 and AAV9 (AAV2AU and AAV9AU). The mutant capsids were packaged with the CBA promoter construct, directly infused into rat striatum, and cellular expression was determined. The insertion of six alanines into AAV2 (AAV2AU) did not alter the cellular transgene expression from that of AAV2EU-CBA or AAV2-CBA (FIGS. 3A-3I, 5A-5D, and 5I); the mCherry positive cells co-localized mostly with NeuN (79.2%±3.8) with limited co-localization with Olig2 (8.3%±3.6) (FIGS. 6A-6I). Also, the insertion of six alanines into AAV9 (AAV9AU-CBA) resulted in a predominant neuronal tropism similar to that seen with AAV9-CBA (FIGS. 3S-3A′). AAV9AU-CBA mCherry expression co-localized with NeuN (89.8%±3.9) while only 2.1%±0.8 of mCherry positive cells co-localized with Olig2 (FIGS. 6J-6R). Notably the increase in mCherry positive neurons with AAV9AU-CBA is significant compared to AAV9EU-CBA (p<0.001). Furthermore, the reduction in mCherry positive oligodendrocytes in AAV9AU-CBA compared to AAV9-CBA was also significant (p<0.005). Surprisingly, when gene expression was driven by the CBh promoter, the six alanine insertions into AAV9 reversed the predominant oligodendrocyte gene expression back to a predominant neuronal gene expression pattern (FIGS. 6S-6W). Together, these results suggest that the glutamates' negative charge in AAV9EU disrupts a capsid-transgene interaction that determines neuronal transgene expression of AAV9-CBA while the neutral alanine insertions reverse the capsid interaction with the CBh promoter that supports oligodendrocyte expression. Again, these results implicate a capsid-promoter interaction in vivo.

### AAV9AU can Transduce Striatal Oligodendrocytes

While there are no published reports of the VP1/2 region influencing AAV gene expression, it was entirely possible that the alanine insertion could possibly have shifted AAV9 capsid binding away from oligodendrocytes. Therefore, we constructed a transgene where gene expression was driven by an oligodendrocyte specific promoter (MBP, myelin basic protein) and then packaged the transgene into AAV9AU or AAV9. Our previous results showed that AAV9AU-CBA mediated transgene expression was predominantly neuronal (FIGS. 6J-6R). However, for both the AAV9AU and AAV9, MBP driven expression of GFP was non-neuronal (FIGS. 7A-7F). Thus, the alanine insertion did not alter the ability of AAV9AU to bind and transduce oligodendrocytes but did prevent oligodendrocyte localized gene expression driven by the constitutive CBh promoter.

AAV9-CBh Oligodendrocyte Preferring Gene Expression is Consistent with Other Transgenes

Due to the size differences in CBA and CBh promoters, the final single stranded constructs differed by approximately 1 kb. To address if packaging size issues influenced the capsid contribution to the change in cellular gene expression, we constructed an AAV9 vector packaged with self-complementary CBh-GFP containing a different poly A (bovine growth hormone) and transgene (GFP) such that the entire transgene was close to the packaging capacity. This vector exhibited both in vivo oligodendrocyte and neuronal gene expression (FIGS. 8A-8F) as was found with the single stranded AAV9 vectors where CBh drove gene expression.

### Discussion:

In the context of AAV9, divergent in vivo cellular gene expression occurred between two similar constitutive promoters, CBA and CBh, even though all other components of the vector were identical. Because in both instances the capsid was identical, differential cellular binding, intracellular trafficking or nuclear localization appear unlikely to be contributing factors. Also, as seen in FIGS. 3A-3J′ both promoters can support gene expression in neurons and oligodendrocytes albeit to differing degrees. Finally, the 1 Kb difference in cassette size does not appear to contribute, because a self-complimentary AAV9 vector with the CBh promoter exhibited oligodendrocyte gene expression in vivo. In marked contrast, no such differences in in vivo gene expression occurred with AAV2 vectors. Therefore, the observed in vivo divergence of AAV9 mediated cellular gene expression do not reflect a general property inherent to all AAV vectors. Clearly, these initial findings suggest that unknown elements of the AAV9 capsid can influence promoter permissiveness within different CNS cellular populations. To date cell specific gene expression has been achieved by manipulation of the capsid, utilization of cell specific promoters or cell specific enhancers (Asokan et al., Mol. Ther. 20:699 (2012); Oh et al., Gene Ther. 16:437 (2009); Dimidschstein et al., Nat. Neurosci. 19:1743 (2016); Blankvoort et al., Curr. Biol. 28:2103 (2018)). Thus, the observed interaction of the AAV9 capsid with constitutive promoter gene expression in vivo reveals a previously unknown, novel property of AAV9 vectors.

Subsequent investigations not only further reinforced the conclusion that the AAV9 capsid can interact with promoter activity but also provided evidence for a specific site that appears directly involved in this capsid/promoter interaction. The insertion of six negatively charged glutamates into the VP1/VP2 junction region had no effect on cellular gene expression in the context of AAV2, even though with the MBP promoter AAV2 supports oligodendrocyte gene expression in the rat CNS (Chen et al., J. Neurosci. Res. 55:504 (1999)). For AAV9 this mutation shifted the balance of CBA mediated gene expression from neurons to oligodendrocyte gene expression. Because the six glutamate insertion exerted no influence on AAV2 neuronal gene expression, this amino acid insertion likely did not cause a general alteration in capsid binding properties. With regard to the potential for some non-specific insertion effect, insertion of six neutral alanines in the same position did not alter the predominant neuronal gene expression by the CBA promoter. However, surprisingly the six alanine insertion into AAV9 reversed the CBh mediated cellular gene expression pattern from oligodendrocytes to a predominant neuronal transduction pattern. Importantly, this reversal in cellular gene expression did not arise from a reduction of cellular capsid binding to oligodendrocytes. When gene expression was driven by an oligodendrocyte specific promoter, both AAV9 and AAV9AU vectors exhibited in vivo gene expression that was confined to oligodendrocytes. Clearly, both AAV9 and AAV9AU gained access to oligodendrocytes, yet in the context of the six alanine insertions CBh mediated gene expression was shifted from oligodendrocytes to neurons. Thus, these amino acid insertions not only validate an AAV9 capsid-promoter interaction, but implicate an area of the capsid that to date has not been associated with modulation of capsid binding or gene expression.

Although the many approaches to AAV capsid engineering focus upon the VP3 capsid protein, several previous studies have provided tangential evidence that the AAV capsid can influence gene expression. Johnson et al. (J. Virol. 84:8888 (2010)) found that mutations in the VP1/VP2 junction in AAV2 caused a significant decrease in in vitro gene expression. Similarly, Aydemir et al. (J. Virol. 90:7196 (2016)) showed that mutations in the “dead zone” of the AAV2 capsid resulted in a lack of gene expression, even though no changes were found in receptor binding, endosome escape, nuclear localization or capsid uncoating. Currently AAV9 vectors have attained a central prominence in clinical gene therapy particularly with regard to single gene disorders such as RPE65 retinal mutations, spinal muscular atrophy and giant axon neuropathy (Bennett et al., Lancet 388:661 (2016)); Mendell et al., N. Eng. J. Med. 377:1713 (2018); Bailey et al., Mol. Ther. Methods Clin. Dev. 9:160 (2018)). In addition, AAV9 capsid sequences comprise a significant proportion of engineered chimeric capsids (Deverman et al., Nat. Biotechnol. 34:204 (2016); Ojala et al., Ther. 26:305 (2018)). Given the rapidly expanding generation of synthetic promoters (Domenger et al., Human Mol. Genetics 28:R3 (2019)), an AAV9 capsid interaction with constitutive promoter activity certainly could alter the actual contributions to cellular gene expression profiles in the CNS.

### Example 2—AAV9 Capsid Interaction with the JeTI Promoter

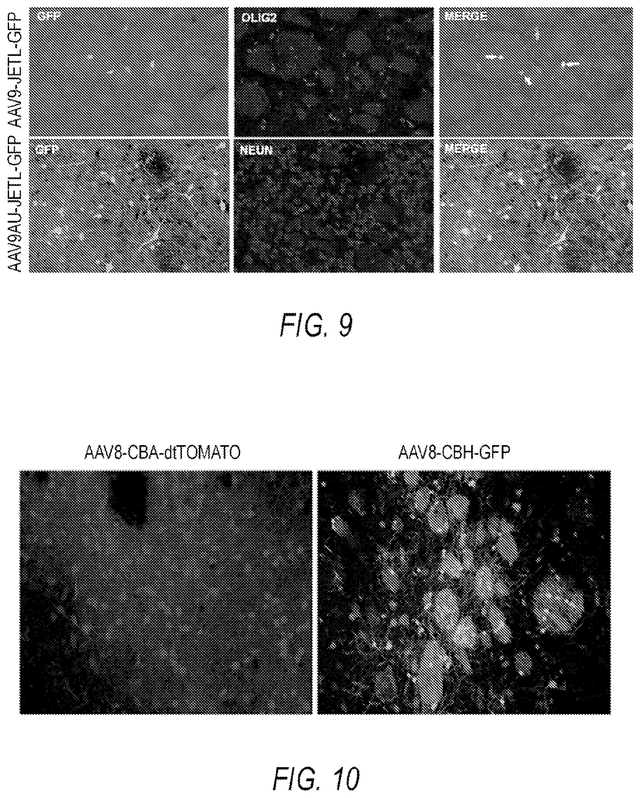

We tested the influence of the AAV9 capsid on synthetic JeTI promoter, also referred to as the UsP promoter (Karumuthil-Melethil et al., Hum. Gene Ther. 27:509 (2016)) driven cellular gene expression in the rat striatum. AAV9-JeTI-GFP or the AAV9AU-JeTI-GFP where six alanines have been inserted into the boundary following amino acid 138 of AAV9 at amino acid 139 of AAV9 VP1/VP2 were generated by methods similar to those in Example 1. Rats received a 3 μl infusion of AAV9-JeTI-GFP or the AAV9AU-JeTI-GFP, by methods similar to those described in Example 1. Two weeks later the rats were perfused, and the striatal tissues sectioned, with gene expression in various cell types determined by methods similar to those described in Example 1. As seen in FIG. 9, AAV9-JeTI gene expression proved prominent in oligodendrocytes (34±4 Olig2 positive cells/section) in comparison to neurons (17±4 NeuN positive cells per section; 67% oligodendrocytes versus 33% neurons). However, in marked contrast the AAV9AU capsid directly reversed the pattern of gene expression to neurons (67±5 NeuN positive cells) versus oligodendrocytes (32±2 Olig2 positive cells; 68% neurons versus 32% oligodendrocytes). This influence of the AAV9AU capsid on the pattern of gene expression agrees with a similar shift in cellular gene expression, when gene expression was driven by a truncated CBA promoter (Powell et al., Mol. Ther. doi.org/10.1016/j.ymthe.2020.03.007 (2020)). Clearly the AAV9 capsid directly influences cellular gene expression from another (synthetic) promoter in the rat striatum.

These experiments can be repeated with different promoters. For instance, the above experiments will be performed with additional promoters, including human synapsin promoter (hSYN1) (McClean et al., Neuroscience Letters, 576 (2014) p′73-′78), a neuron specific promoter, and also a ubiquitous cytomegalovirus promoter (CMV).

The promoters will be engineered into the AAV9 reporter constructs described in Examples 1 and 2, and used to generate rAAV9 virus particles with capsid sequences with and without the above six alanine insertion into amino acid 139 of AAV9 VP1/VP2. The resulting rAAV9 vector particles will be administered to the rat striatum, which will then be analyzed for reporter gene expression in various cell types (neuron, oligodendrocyte). The amounts of gene expression in the different cell types will be determined and quantitated by the methods described directly above and/or in Example 1. A similar shift in the pattern of gene expression (e.g., to increased preferential expression in neurons) will be identified.

Similarly, these experiments can also be performed in different tissues outside of the central nervous system (e.g., using cellular markers specific for the different cell types for other rAAV expression preference.

### Example 3—AAV8 CBA Versus CBh Transduction in the Rat Striatum

rAAV8 virus particles having different promoters (CBA and CBh) were prepared by methods similar to those described in Example 1. The reporter transgene used was dtTomato. The rAAV virus particles were injected into the rat striatum and 2 weeks later reporter gene expression was imaged using confocal microscopy. As seen in FIG. 10, when gene expression was driven by the CBA promoter, the vast majority of the dtTomato positive cells exhibited clear neuronal characteristics. In marked contrast, when gene expression was driven by the CBh promoter, many of the cells exhibited clear oligodendrocyte cellular characteristics including GFP positive myelin patches (FIG. 10). This study indicates that, like AAV9 capsids, the AAV8 capsid can influence cellular gene expression via promoter permissiveness in the rat CNS.

These experiments can be repeated with different AAV serotypes. A similar effect of capsids on promoter permissiveness will be identified. Similarly, these experiments with different AAV serotypes can also be performed in different tissues outside of the central nervous system (e.g., using cellular markers specific for the different cell types for other rAAV expression preference.

### Example 4—AAV Capsid-Promoter Interactions in the Brain Translate from Rat to the Non-Human Primate

### Introduction

Adeno-associated virus (AAV) vectors have achieved a prominent position in CNS clinical trials particularly with respect to the use of AAV serotype 9 (AAV9) for single gene disorders, such as spinal muscular atrophy and giant axon neuropathy (Mendell et al., N. Engl. J. Med. 377:1713 (2017); Bailey et al., Mol. Ther. Methods Clin. Dev. 9:160 (2018)). From a basic research perspective, many AAV vectors have been used to target precise neuronal populations for optogenetic and chemogenetic manipulation, an approach that has revealed complex neuroanatomical connections and novel insights into functional dynamics (El-Shamayleh et al., Proc. Natl. Acad. Sci. USA 116:26195 (2019); Galvan et al., J. Neural Transm. (Vienna) 125:547 (2018); Galvan et al., J. Neurosci. 37:10894 (2017); Haggerty et al., Mol. Ther. Methods Clin. Dev. 17:69 (2020)). In general, achieving cell selective transduction and gene expression has involved manipulation of the AAV capsid and utilization of cell specific promoters or enhancers (Asokan et al., Mol. Ther. 20:699 (2012); Dimidschstein et al., Nat. Neurosci. 19:1743 (2016); Grimm et al., Hum. Gene Ther. 28:1075 (2017); Tervo et al., Neuron 92:372 (2016)), where these elements have been thought to be mutually exclusive.

The work described in Examples 1-3 established that the AAV9 capsid exhibits a previously unknown novel property within the rodent central nervous system (CNS), namely the ability of a capsid-promoter interaction to influence cell specific transgene expression in vivo (Powell et al., Mol. Ther. 28:1373 (2020)). Using identical transgene cassettes packaged in the AAV9 capsid, the chimeric CMV-chicken β-actin promoter, CBA, supported dominant neuronal gene expression in the rat striatum, while a truncated CBA promoter, a novel hybrid form of the CBA promoter, CBh, exhibited a significant shift in transgene expression to oligodendrocytes. Moreover, six glutamate or six alanine insertions into capsid proteins VP1/VP2 reversed the anticipated cellular transgene expression driven by CBA or CBh, respectively. These studies provide the first demonstration of an AAV capsid-promoter interaction that influences cell type specific expression. This general principle of a capsid-promoter interaction explains divergent results observed in previous studies. For example, Haberman et al. (Mol. Ther. 6:495 (2002)) reported that two AAV vectors with similar promoters driving therapeutic gene expression exhibited diametrically opposed results with regard to in vivo seizure attenuation. When therapeutic gene expression was driven by a CMV promoter containing a CMV immediate early enhancer, in vivo seizure sensitivity was significantly attenuated. When the same therapeutic gene expression was driven by a minimal CMV promoter (without enhancer) in the context of a TET-off element, in vivo seizure sensitivity was significantly heightened. The basis for this dichotomy was revealed by mixing the two rAAV2 vectors where CMV drove LacZ expression and Tet-OFF-CMV drove GFP expression. After CNS infusion, 41% of the transduced neurons expressed GFP alone, 24% expressed LacZ alone, and 35% were positive for both reporter genes. In light of our demonstration of an AAV capsid-promoter interaction (Powell et al., Mol. Ther. 28:1373 (2020)), re-interpretation of the findings by Haberman et al. (Mol. Ther. 6:495 (2002)) indicates that an rAAV2 capsid interaction with one or both of the promoters caused divergent patterns of neuronal gene expression. Thus, the observed interactions and resultant effects of capsid and promoter reported herein for AAV8 and AAV9 further extend to additional AAV serotypes, and are likely conserved across all serotypes to some degree to influence cellular expression levels.

Given that numerous AAV capsid libraries utilize constitutive or cell type specific promoters as part of the selection criteria (Asokan et al., Mol. Ther. 20:699 (2012)), we aimed to expand on previously reported capsid-promoter interactions in rats and determine whether this phenomenon translates to the non-human primate brain. We report herein that, as with the CBA and Cbh promoters, the AAV9 capsid also interacts with the JetI synthetic promoter (Karumuthil-Melethil et al., Hum. Gene Ther. 27:509 (2016)) to alter cellular transgene expression in the rat brain. Also, based upon the recent observation of a AAV capsid-promoter interaction (Powell et al., Mol. Ther. 28:1373 (2020)), we decided to retrospectively look for potential evidence of this interaction across viral injections made in primates. For this, we aggregated primate neuroanatomical data from two labs whose focus was on a single capsid, rAAV2-Retro, with injections placed within the well-understood visuomotor circuitry of the primate brain. From the available data, we were able to make revealing comparisons, where rAAV2-Retro carrying either the human synapsin (hSyn) or a hybrid chicken beta actin (CAG) promoter was infused into either the macaque frontal eye field or superior colliculus. In general, the neuroanatomical underpinnings of these visuomotor structures is phylogenetically conserved and their connectivity has been established across a broad number of species using autoradiographic and conventional tracers. Thus, retrograde transduction patterns were compared across viral injections using the well-established afferent connections with the frontal eye field (Huerta et al., J. Comp. Neurol. 293:299 (1990); Huerta et al., J. Comp. Neurol. 265:332 (1987); Leichnetz et al., Rev. Oculomot. Res. 2:365 (1988)) and superior colliculus (Kurimoto et al., Neurosci. Res. 22:57 (1995); May et al., Neuroscience 36:305 (1990); May et al., Hall WC. Exp. Brain Res. 65:200 (1986); Kawamura et al., Neuroscience 7:1673 (1982); Hirai et al., Exp. Brain Res. 48:1 (1982)) in monkeys. Results from both species supported the hypothesis that cell specific capsid-promoter interactions influence cellular transgene expression following retrograde transport. These findings provide a potential explanation for conflicting in vivo neural circuit expression patterns in previous experiments that involved different promoters (Cushnie et al., J. Neurosci. Methods 345:108859 (2020)) and AAV capsids and point to the importance of considering capsid-promoter interactions in translation to clinical applications. Finally, these findings raise questions regarding the basis for cellular specificity of many engineered chimeric AAV capsids, as well as the more extensively studied conventional AAV capsids.

### Materials and Methods

### Rodents:

Rodent Virus production: Viruses were produced in HEK293 cells as previously described (Weinberg et al., Mol. Ther. 25:928 (2017)). Briefly, polyethylenimine max (PEI) was used for the triple transfection of a cap and rep plasmid (pGSK2/9 and pGSK2/9AU), the pXX6-80 helper plasmid, and the transgene plasmid (pJetI-GFP, gift from Dr. Steve Gray, UT Southwestern). Cells were harvested 48 hr post-transfection, and the virus was purified by cesium chloride ultracentrifugation. After identifying peak fractions by qPCR, the virus was dialyzed into 1XPBS/NaCl/D-Sorbitol. Titers were calculated by qPCR according to established procedures using a LightCycler 480 instrument and ITR primers. The individual titers were 2.9×10¹¹ vector genomes/ml for scAAV9-JetI-GFP and 8.4×10¹¹ vector genomes/ml for scAAV9AU-JetI-GFP.

Animals and Stereotactic Infusions: All of the animals included in the current study were male Sprague-Dawley rats (Charles River Laboratories) weighing between 200 and 300 grams at the time of intracranial injections. Animals were maintained on a 12-hr light-dark cycle and had free access to water and food. Animal care and surgical procedures were in accordance with the NIH Guide for the Care and Use of Laboratory Animals, and all procedures received prior approval by the University of North Carolina Institutional Animal Care and Usage Committee.

Virus vector infusions were performed as previously described (Weinberg et al., Mol. Ther. 25:928 (2017)). First, animals were anesthetized using pentobarbital (50 mg/kg, IP) and placed into a stereotactic frame. Using a 32G stainless steel injector and a Hamilton infusion pump, animals received 3 μl of each vector over 15 min into each side of the striatum (0.5 mm anterior to bregma, 3.5 mm lateral, and 5.5 mm vertical, according to the atlas of Paxinos and Watson (Paxinos et al., The rat brain in stereotaxic coordinates. Academic Press, San Diego (1998)). The injector was then left in place for 3 min post-infusion, to allow time for the virus to diffuse from the injection site.

Histology and Microscopy: Two weeks after AAV vector infusion, animals received an overdose of pentobarbital (100 mg/kg, IP), and they were perfused transcardially with ice-cold 100 mM PBS (pH 7.4), followed by 4% paraformaldehyde in PB (pH 7.4). Brains were post-fixed 12-48 hr at 4° C. in the paraformaldehyde-PB, then 40-μm coronal sections were cut using a vibrating blade microtome for subsequent immunofluorescence. To determine fluorescent transgene (GFP) cellular co-localization, tissue sections were incubated in a blocking solution with either cellular marker antibodies: NeuN (1:500, Chemicon), Olig2 (1:250, Abcam) or GFAP (1:2000, Dako). Following incubation at 4° C. for 48-72 hr in primary antibodies, the sections were rinsed three times with PBS and blocked again for 45 min at room temperature. Subsequently, the tissue sections were incubated in either Alexafluor 488 or 594-conjugated goat anti-rabbit IgG or goat anti-mouse (Invitrogen)(diluted 1:500 in 10% goat serum/PBS) for 1 hr at 4° C. Rinsed sections were mounted, and fluorescence was visualized using an Olympus FV3000RS confocal microscope in the UNC Neuroscience Center Microscopy Core. Transgene fluorescence co-localization was determined on the z stacks. Individual GFP positive/marker positive cells were counted across 4 sections separated by 80 microns for each vector condition. Significant differences between the total number of neuron or oligodendrocyte positive cells was determine by a two tailed students t-test.

### Primates:

Viral vectors: Viral vectors were purchased from commercial sources. Titers, injection locations and parameters are reported in Table 5.

Animals and Stereotactic Infusions: All methods were approved by either the University of California, Los Angeles, or Duke University IACUCs and were performed in accordance with the NIH Guidelines for Animal Care and Use.

Frontal Eye Field Injections: Case 1 participated in transcranial electric and magnetic stimulation studies (Lee et al., Neuropsychopharmacology 42:1192 (2017); Lee et al., IEEE Trans. Biomed Eng 62:2095 (2015); Peterchev et al., IEEE Trans. Biomed Eng. 55:257 (2008); Peterchev et al., Neuropsychopharmacology 40:2076 (2015)). In cases 1-3, animals received a prophylactic dose of corticosteroids (Dexamethasone (2.0 mg/kg, IM) or Solu-Medrol (15.0 mg/kg, IM)) the day before surgery and this dosage was tapered over two weeks, post-operatively. On the surgery date, animals were sedated with ketamine hydrochloride (3.0 mg/kg, IM) and dexdomitor (0.075 mg/kg, IM), then intubated. An anesthetic plane was subsequently maintained for the duration of surgery using a 1-3% isoflurane/oxygen mix.

The animal was placed into a stereotaxic apparatus (Kopf Instruments, Tujunga, Calif.). All surgical procedures were carried out under aseptic conditions. The scalp was thoroughly cleaned using betadine and chlorhexidine scrubs, followed by 200 proof ethanol. During surgery, vital signs were monitored and maintained within normal limits by a trained veterinary technician. Before an incision was made and after final suturing, a cutaneous injection of 0.25% Bupivacaine (0.5-1.0 ml/<4 mg) was administered along the incision line.

For frontal eye field injections, a midline incision was made, soft tissues were retracted to visualize the skull. Using stereotaxic coordinates, a hole was trephined above the frontal eye field. A durotomy was performed to visualize the underlying cortex. A Hamilton syringe was held in a micromanipulator at either a 90° angle (Table 5, Case 1) with respect to the horizontal plane or at a 45° angle (Table 5, Case 2&3), with the tip angled medially. The surface of the frontal eye field sits at approximately 45° in the horizontal plane thus, by angling the needle at 45°, the track was orthogonal to the cortical surface. For cases 1&3, the needle was advanced ˜5 mm from the surface, where 2 μl of rAAV2-Retro was deposited at ˜1 μl/min rate. The solution was allowed to diffuse from the injection site for 5 min. Next, the syringe was drawn up 1 mm and another 1-2 μl injection was made, followed by a second 5-min waiting period before withdrawing the needle. This procedure was replicated at multiple sites along the genu of the arcuate sulcus. For case 2, the needle was advanced to ˜5 mm from the cortical surface, then drawn up 2 mm and a single 5 μl injection was made at 0.5 μl/min within the FEF.

Following injections, the dura was sutured back together, and the bone flap was replaced, then the muscle was sutured back to its insertion points. Finally, the skin was reapproximated with suture. For postoperative analgesia, animals received one dose of buprenorphine SR (0.2 mg/kg, IM).

Superior Colliculus Injections: Cases 4-6 were involved in prior psychophysical and electrophysiological studies (Grimaldi et al., J. Neurophysiol. 120:2614 (2018); Odegaard et al., Proc. Natl. Acad. Sci. USA 115:E1588 (2018); Crapse et al., Neuron 97:181 (2018)). Thus, these animals had existing chambers which provided access to the superior colliculus. Using electrophysiologically identified coordinates, a 10 μl Hamilton syringe or a custom injectrode was used to inject a total of: 3 μl at 0.1 μl/min in a single location within the SC (Case 4&5) or 9 μl at 0.1 μl/min in 3 locations throughout the SC (Case 6). At each site, a 1 μl injection was made at 3 depths.

Histology and Microscopy: Following a survival period (Table 5), animals were sedated with ketamine hydrochloride (3.0 mg/kg, IM) then deeply anesthetized with sodium pentobarbital (50.0 mg/kg, IP). Once the animals were totally areflexic, they were transcardially perfused with 2-4 L of chilled 0.1 M, pH 7.4 phosphate buffered saline (PBS), followed by 4 L of 4% paraformaldehyde in 0.1 M, pH 7.4 PBS. Next, the brain was blocked in the frontal plane using a stereotaxic apparatus and post-fixed in 4% paraformaldehyde in 0.1 M, pH 7.4 PBS at 4° C. for 24-48 hr. Blocks were then cryoprotected in 30% sucrose at 4° C. Afterwards, blocks were cut using a freezing stage, sliding microtome (American Optical Company, Buffalo, N.Y.) and sections stored in PBS at 4° C.

For immunofluorescence amplification, free floating sections were incubated in immunoblocking serum consisting of 1% bovine serum albumin/0.1% Triton X-100 in PBS for 2 hr at room temperature. Following a rinse, sections were incubated in rabbit anti-GFP in PBS (1:200; Abcam #5450) for 24 to 48 hr at 4° C. Next, sections were rinsed and placed in a secondary antibody solution consisting of 1:385 donkey anti-rabbit IgG antibody conjugated to Alexa Fluor 488 (Jackson ImmunoResearch #705-545-147) in 2% normal donkey serum in PBS for 2 hr at room temperature.

For immunohistochemical visualization of the green fluorescent family of proteins, a detailed protocol has previously been published (Bohlen et al., Front. Neuroanat. 13:84 (2019)). Briefly, free-floating sections first underwent a blocking step to inhibit endogenous peroxidase activity. Sections were moved to a 0.25% solution of Triton X-100 in PBS, then transferred to a 1% BSA/0.25% Triton X-100 in PBS solution. They were incubated in biotinylated goat anti-GFP (˜1:200; Rockland 600-106-215) in a 1% BSA/0.25% Triton X-100 in PBS solution for 1-3 hr at room temperature, followed by ˜24-48 hr at 4° C. After being rinsed in PBS, they were incubated in biotinylated rabbit anti-goat IgG secondary antibody (Vector Laboratories, PK6105) for 1.5 hr at room temperature. Sections were then transferred to an avidin-biotin-horseradish peroxidase complex (ABC) solution (Vector Laboratories, PK6105) for 1 hr at room temperature. Following another 15-min PBS wash, sections were placed in 0.5% diaminobenzidine (DAB)/0.01% cobalt chloride/0.01% nickel ammonium sulfate in PBS solution for 20 min. Subsequently, 0.3% H₂O₂ was added and allowed to react with the DAB for 15-30 min to produce a dark brown-black reaction product in locations where cells contained the fluorescent protein. Sections were then mounted on gelatinized glass slides and left to air dry overnight. Dry mounted sections were counter stained with thionin. In all cases, sections were then dehydrated in graded alcohol baths and coverslipped using Cytoseal 60.

Sections were drawn using a Bausch & Lomb microprojection microscope for structural anatomy, then a Zeiss Axiolmager 2 with an affixed drawing tube was used to plot the locations of labeled cells over the anatomical drawing. For one case (Case 5), the only available tissue was fluorescence. Sections from this case were scanned using a Zeiss Axioscan. The digital scan of the entire section was imported into CorelDRAW 2020 (Corel Corp. Ottawa ON, CA), in which the anatomical outlines were drawn, and the locations of labeled cells were marked. Brightfield and Fluorescent photomicrographs were taken using a Zeiss Axio Scan.Z1.

### Results

AAV9 Capsid Interactions with a Synthetic Promoter Determines Cellular Transgene Expression in the Rat Striatum

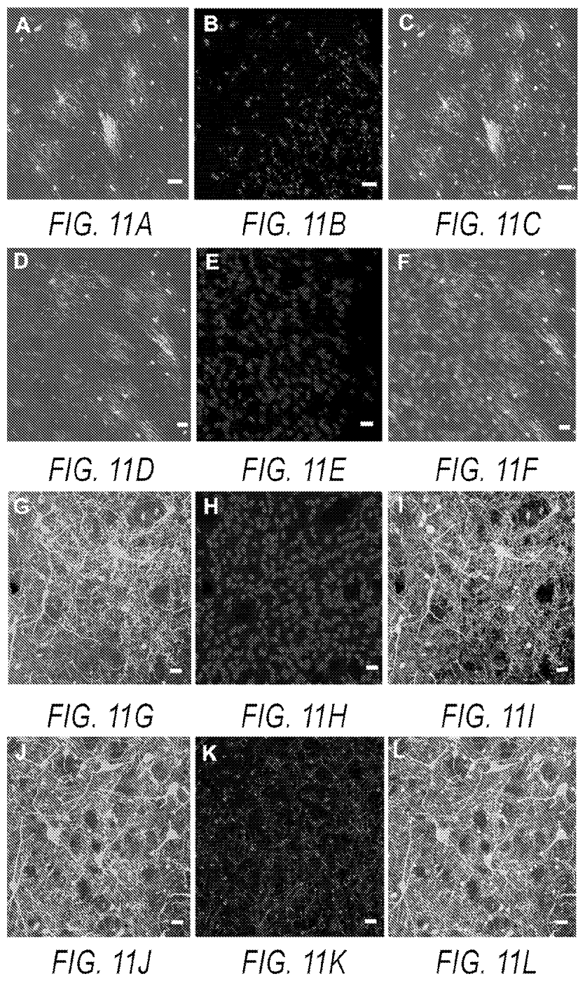

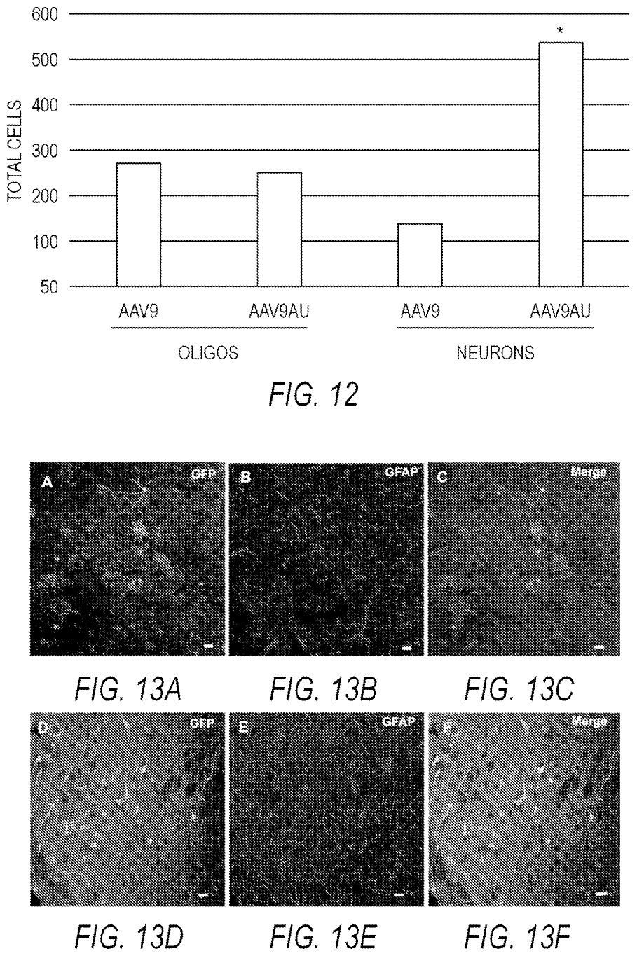

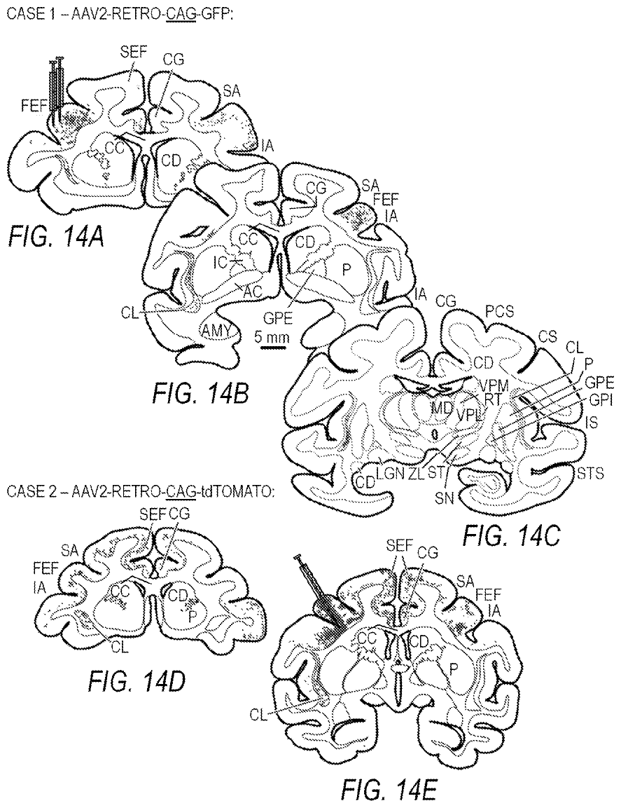

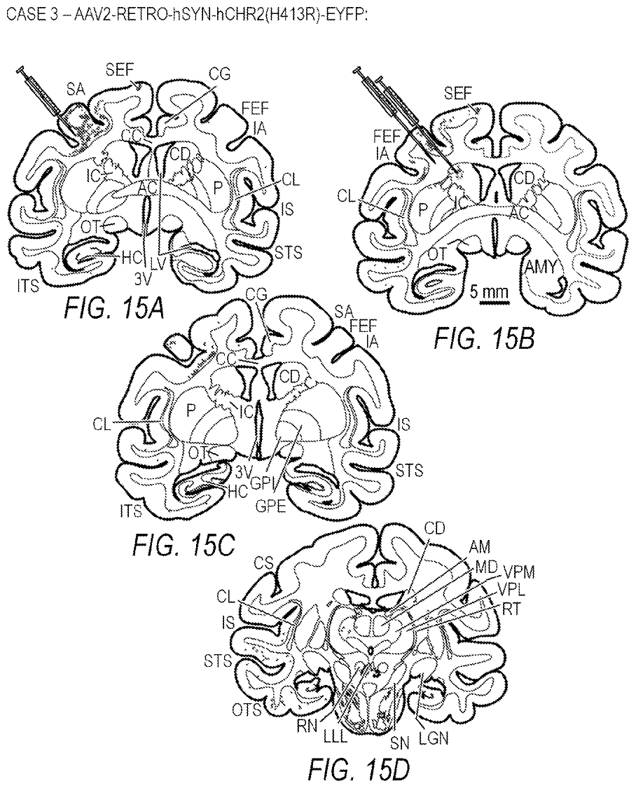

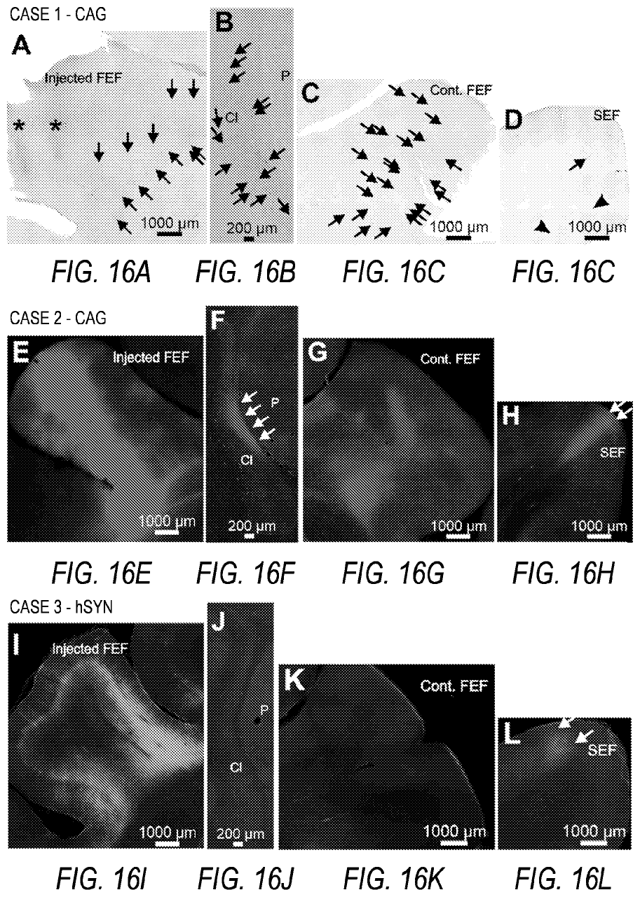

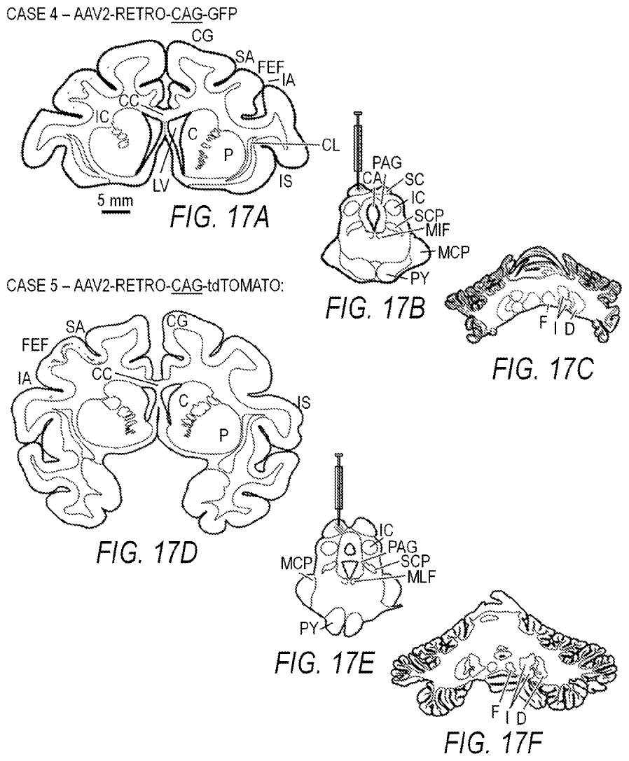

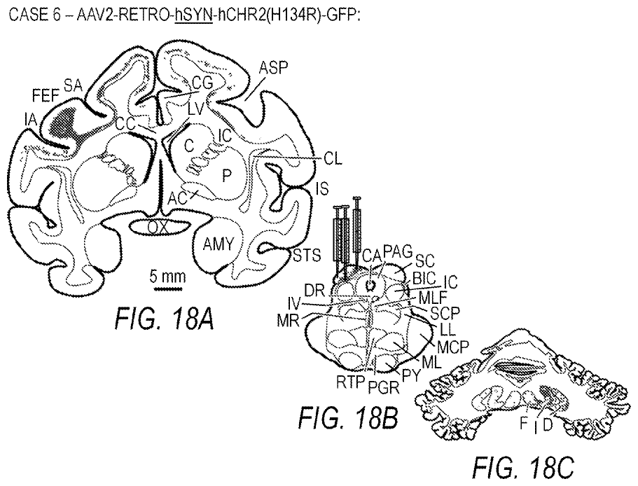

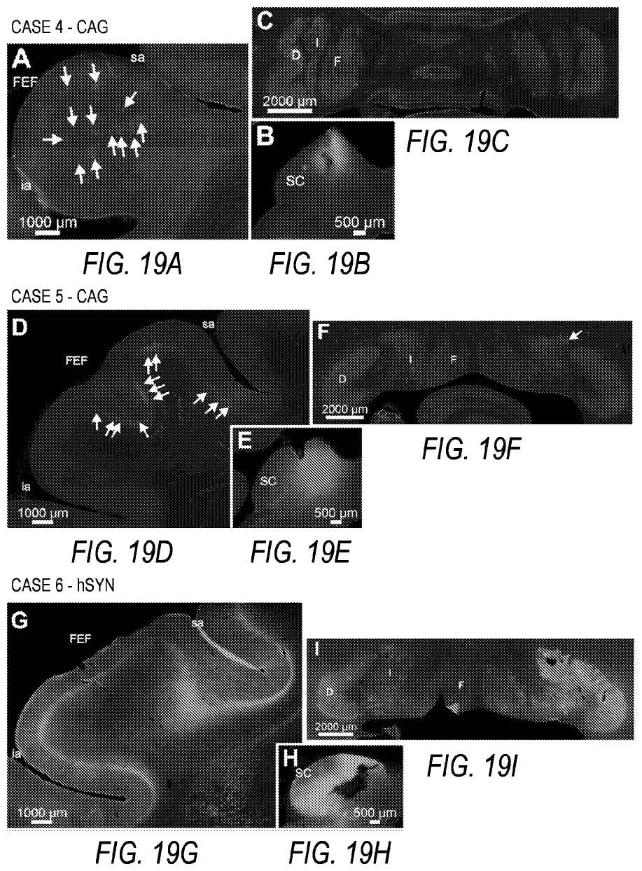

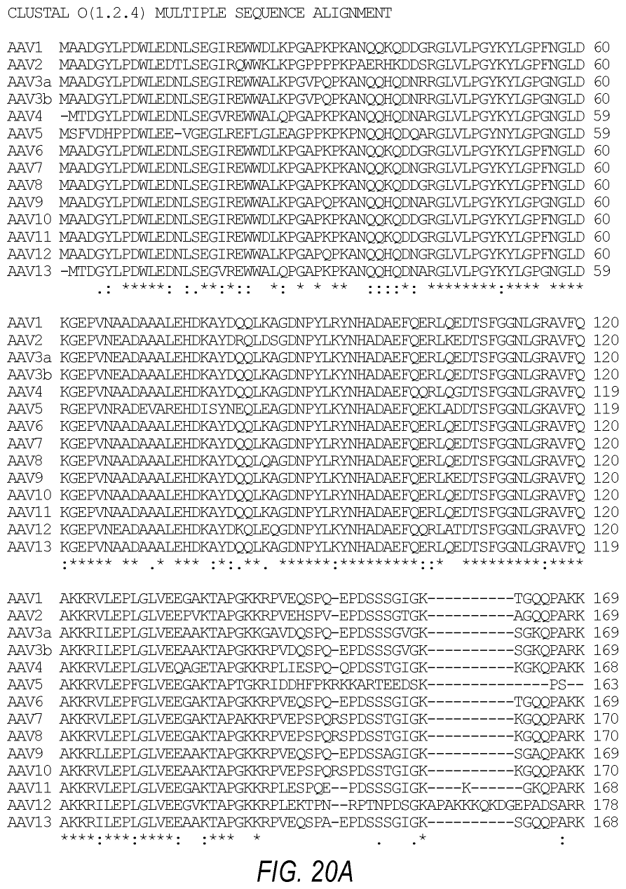

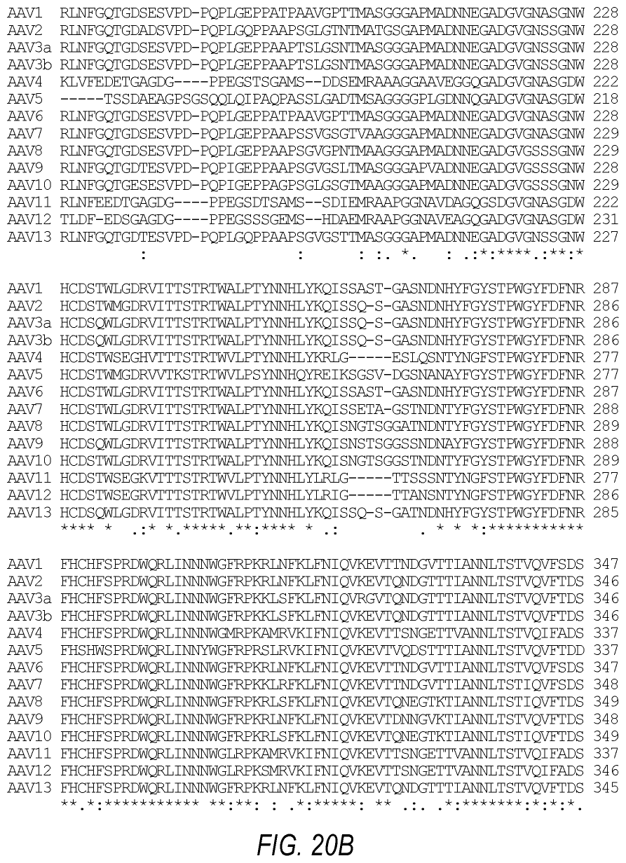

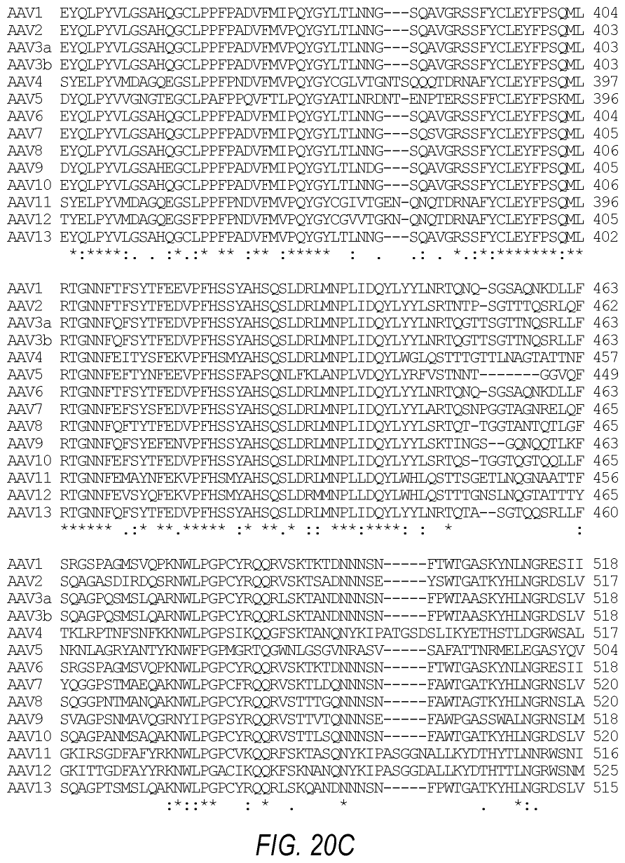

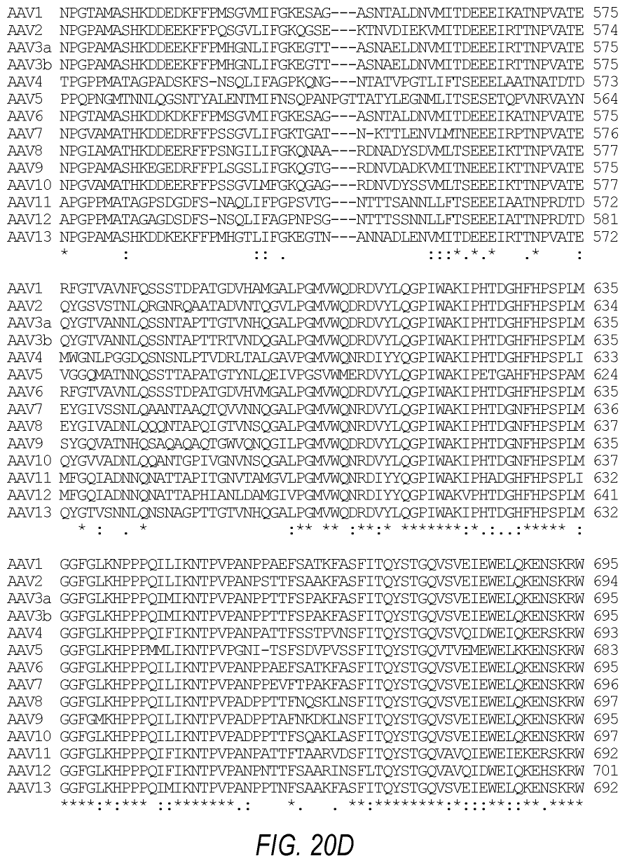

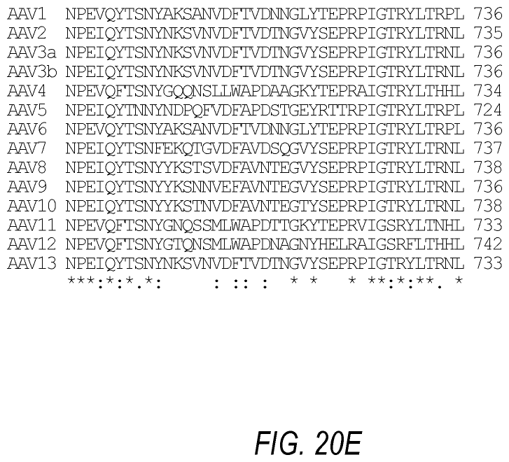

In order to verify the capsid-promoter interactions observed with the CBA and Cbh promoters in AAV9 also occur with other promoters, we tested the influence of the AAV9 capsid on a synthetic JetI promoter (Karumuthil-Melethil et al., Hum. Gene Ther. 27:509 (2016)) driving cellular transgene expression of GFP in the rat striatum. Rats received a 3 μl infusion of AAV9-JetI-GFP or the AAV9AU-JetI-GFP where six alanines had been inserted into amino acid 139 of AAV9 VP1/VP2. Two weeks later, the rats were perfused, the brains sectioned, processed immunohistochemically and the region containing the injection site was analyzed. As seen in FIGS. 11A-11C, the green AAV9-JetI GFP expression is prominent in oligodendrocytes identified by the red fluorescent marker for anti-Olig2 (34±4 Olig2 positive cells/section). By comparison, relatively few neurons co-localized with the red fluorescent marker attached to anti-NeuN (17±4 NeuN positive cells per section) (FIGS. 11D-11F). In marked contrast, the insertion of six alanine residues into AAV9 VP1/2 (AAV9AU) significantly altered the AAV9-mediated cellular expression pattern. FIGS. 11G-11I show that there are numerous green fluorescing GFP neurons that co-localize with the red anti-NeuN signal (67±5 NeuN positive cells/section), while the number of GFP positive cells co-localizing with red anti-Olig2 positive oligodendrocytes remained similar to that after AAV9 transduction (FIGS. 11J-11K)(32±2 Olig2 positive cells). FIG. 12 shows that the AAV9AU vectors significantly increased the total number of GFP-NeuN positive cells versus AAV9 vectors but did not alter the total number of GFP-Olig2 positive cells. This influence of the AAV9AU capsid on cellular GFP expression agrees with a similar shift in cellular transgene expression we observed in the context of the CBh promoter (Powell et al., Mol. Ther. 28:1373 (2020)). No astrocyte co-localization was found for either promoter (FIGS. 13A-13F). Thus, the data show that the AAV9 capsid directly influences cellular transgene expression from another constitutive, synthetic promoter in the rat striatum. We anticipate that these phenomena are more widespread than the limited number of cassettes and capsids we have selected. Given a number of disparate findings for non-human primate investigations (Cushnie et al., J. Neurosci. Methods 345:108859 (2020)), we therefore conducted retrospective studies on visual field AAV transduction in non-human primates.

rAAV2-Retro Promoter Dependent Frontal Eye Field Retrograde Transgene Expression in the Non-Human Primate

The rAAV2-Retro vector has provided a powerful tool for neuronal circuit investigations in the CNS across a number of species (Tervo et al., Neuron 92:372 (2016); Weiss et al., bioRxiv 2020.2001.2017.910893 (2020)). The present study investigated the pattern of retrograde labeling with rAAV2-Retro using either a constitutive promoter, CAG, or a neuron-specific promoter, hSyn. Retrograde transduction patterns were compared across injections using the well-established afferent connections with the frontal eye field in monkeys (Huerta et al., J. Comp. Neurol. 293:299 (1990); Huerta et al., J. Comp. Neurol. 265:332 (1987); Leichnetz et al., Rev. Oculomot. Res. 2:365 (1988)). Intraparenchymal infusion of rAAV2-Retro-CAG-GFP, rAAV2-Retro-CAG-tdTomato, or rAAV2-Retro-hSyn-hChR2(H134R)-EYFP into the frontal eye field (FEF) resulted in substantial, local neuronal labeling (determined when labeled somata were within the plane of section) within the injected frontal eye field (FIGS. 14A, 14E; FIGS. 15A-15C; FIGS. 16A, 16E, 16I). In all three cases, a limited number of retrogradely labeled neurons was observed within the ipsilateral cingulate gyms (CG, FIGS. 14A, 14D-14E; FIGS. 15B, 15C) and ipsilateral supplementary eye field (FIGS. 14A, 14D-14E; FIGS. 15B;15C; FIGS. 16D, 16H, 16L). However, a comparison of transduction patterns between the different promoters revealed unanticipated stark differences. The rAAV2-Retro-CAG-GFP produced retrograde labeling of a large number of neurons within the claustrum (FIGS. 14A-14C, 14D-14E; FIGS. 16B, 16F). It also retrogradely labeled corticocortical neurons within the frontal eye field contralateral to the injection site (FIGS. 14A-14B, 14D-14E; FIGS. 16C, 16G). In contrast, injections of rAAV2-Retro-hSyn-hChR2(H134R)-EYFP failed to provide the same pattern of neuroanatomical expression within either the claustrum (FIGS. 15A-15D; FIG. 16J) or the contralateral frontal eye field (FIGS. 15A-15C; FIG. 16K). Additionally, all of the injected vectors failed to show strong evidence of transgene expression within thalamocortical neurons in the mediodorsal thalamus, a well-established connection to the frontal eye field (Leichnetz et al., Rev. Oculomot. Res. 2:365 (1988); Huerta et al., J. Comp. Neurol. 253:415 (1986); Tian, J. Neurosci. 17:9233 (1997)). These results are summarized in Table 6 and extend our rodent observations of capsid/promoter specific expression profiles to the non-human primate animal model.

### Promoter Dependent Superior Colliculus Retrograde Transgene Expression in the Non-Human Primate

The cereberotectal connection is phylogenetically conserved and well established using autoradiographic and conventional tracer techniques (Rat: Kurimoto et al., Neurosci. Res. 22:57 (1995); Squirrel: May et al., Hall WC. Exp. Brain Res. 65:200 (1986); Cat: Kawamura et al., Neuroscience 7:1673 (1982); Hirai et al., Exp. Brain Res. 48:1 (1982); Monkey: May et al., Neuroscience 36:305 (1990)). Furthermore, the superior colliculus is a second important locus in the control of eye movements, so injections of rAAV2-Retro constructs carrying either the hSyn or CAG promoters were placed into the superior colliculus of three monkeys. Following electrophysiological mapping of the superior colliculus, a small injection of rAAV2-Retro-CAG-GFP or rAAV2-Retro-CAG-tdTomato was placed caudally within the superior colliculus of two animals (FIGS. 17B, 17E; FIGS. 19B, 19E), and multiple injections of rAAV2-hSyn-hChR2(H134R)-GFP were made in the colliculus of a third animal (FIG. 18B; FIG. 19H). Anatomical assessment of transduction revealed local neuronal labeling around the injection sites for all three cases (FIGS. 17B, 17E; FIG. 18B; FIGS. 19B, 19E, 19H). Inspection of the frontal eye fields from all three cases revealed the presence of retrogradely labeled corticotectal neurons within the frontal eye field ipsilateral to the injection site (FIGS. 17A, 17D; FIG. 18A; FIGS. 19A, 19D, 19G). Additionally, in Case 6, the likely combination of the larger injection volume and longer survival duration produced far more labeled cells in the frontal eye field, as well as labeling in adjacent cortical areas (Table 5, Case 6; FIGS. 17A, 18A, 19G). However, inspection of the deep cerebellar nuclei revealed a surprising lack of cerebellotectal neurons expressing fluorescent proteins in the rAAV2-Retro-CAG cases (FIGS. 17C, 17F; FIGS. 19C, 19F). In contrast, the deep cerebellar nuclei of the animal injected with rAAV2-Retro-hSyn-hChR2(H134R)-GFP was heavily labeled (FIG. 18C; FIG. 19I). The labeled cerebellotectal neurons were found in all the deep cerebellar nuclei contralateral to the injection site. These results are summarized in Table 7, and again indicate that the AAV capsid while permissive for broad cell type infectivity demonstrates cell type specific transduction solely based on the capsid/promoter combination.

TABLE 5
Basic monkey parameters

| | | | | | | Injection | Expression |
|---|---|---|---|---|---|---|---|
| Case - | | Injection | | | Titer | Volume | Latency |
| Lab ID | Species | Site | Construct | Source | (vg/mL) | (μl) | (Days) |
| 1 - | Rhesus | FEF | rAAV2-Retro- | UNC | 1.8 × 10¹² | 18 | 28 |
| M17-04 | Macaque | (Broadman | CAG-GFP | | | | |
| | | Area 8) | | | | | |
| 2 - | Cynomolgus | FEF | rAAV2-Retro- | UNC | 2.6 × 10¹² | 5 | 147 |
| M20-01 | Macaque | (Broadman | CAG-tdTomato | | | | |
| | | Area 8) | | | | | |
| 3 - | Cynomolgus | FEF | rAAV2-Retro-hSyn- | AddGene | 1.1 × 10¹³ | 20 | 64 |
| M19-03 | Macaque | (Broadman | hChR2(H134R)- | | | | |
| | | Area 8) | EYFP | | | | |
| 4- | Rhesus | Superior | rAAV2-Retro- | UNC | 1.8 × 10¹² | 3 | 51 |
| M19-07 | Macaque | Colliculus | CAG-GFP | | | | |
| 5 - | Rhesus | Superior | rAAV2-Retro- | UNC | 3.8 × 10¹² | 3 | 98 |
| M20-02 | Macaque | Colliculus | CAG-tdTomato | | | | |
| 6- | Rhesus | Superior | rAAV2-Retro-hSyn- | Salk | 2.5 × 10¹³ | 9 | 264 |
| M18-11 | Macaque | Colliculus | hChR2(H134R)-GFP | | | | |

TABLE 6
Summary of FEF data

| | Injection | | | | | |
|---|---|---|---|---|---|---|
| | Site | | | Contralateral | MD | |
| Construct | Labeling | SEF | Claustrum | FEF | Thal. | FIG. Ref. |
| rAAV2-Retro-CAG-GFP | Yes | Yes | Yes | Yes | No | FIG. 14A-C |
| rAAV2-Retro-hSyn-hChR2(H134R)-EYFP | Yes | Yes | No | No | No | FIG. 15A-D |

TABLE 7
Summary of SC data

| | | | Deep | |
|---|---|---|---|---|
| | Injection Site | | Cerebellar | |
| Construct | Labeling | FEF | Nuclei | FIG. Ref. |
| rAAV2-Retro-CAG-GFP | Yes | Yes | No | FIG. 16A-C |
| rAAV2-Retro-CAG-GFP | Yes | Yes | No | FIG. 16D-F |
| rAAV2-Retro-hSyn-hChR2(H134R)-GFP | Yes | Yes | Yes | FIG. 17A-C |

### Discussion

The present study expands upon the novel observation that AAV capsid-promoter interactions can directly influence AAV cell specific gene expression within the rat CNS presented in Examples 1-3 above (Powell et al., Mol. Ther. 28:1373 (2020)). In addition to CBA and Cbh promoters, the present findings extend this observation by establishing an AAV9 capsid-promoter interaction with the clinically utilized JetI, a ubiquitous minimal synthetic promoter. In the rat striatum AAV9-JetI-GFP vectors exhibited predominantly oligodendrocyte gene expression over neuronal gene expression. However, insertion of 6 alanines into VP1/VP2 of the AAV9 (AAV9AU) capsid as previously described (Powell et al., Mol. Ther. 28:1373 (2020)) significantly shifted the gene expression from oligodendrocytes to neurons. This AAV9AU capsid induced shift in cellular gene expression recapitulates a similar AAV9AU capsid influence on cellular gene expression previously found for the Cbh promoter (Powell et al., Mol. Ther. 28:1373 (2020)). Critical to these experiments is the fact that Powell et al. also established that the alanine insertion did not alter the ability of AAV9 to gain access to oligodendrocytes. As a result of these previous published data combined with the above JetI combination, it is clear that this in vivo AAV9 capsid-promoter interaction translates yet to another promoter (i.e., synthetic) and may be a widespread phenomenon subject to various AAV capsid/promoter combinations.

Previous efforts looking at AAV capsid libraries has demonstrated novel new reagents but in some cases species specific isolation of AAV vectors that do not translate to larger animal models (Matsuzaki et al., Neurosci. Lett. 665:182 (2018)). The present non-human primate findings further suggest that this CNS capsid-promoter interaction not only occurs in another species, but also with a different AAV capsid. All vector constructs were packaged with the rAAV2-Retro capsid, so the efficiency of in vivo retrograde transport would be expected to be the same across the two different promoter constructs. For the FEF injections, the CAG expression vectors supported robust retrograde expression in the contralateral FEF and ipsilateral claustrum, while the hSyn expression vectors did not exhibit retrograde gene expression in these structures. One possible explanation for these results could involve different virus titers and survival times (Table 5). However, for the FEF injections, the CAG vector injections were lower in titer and shorter in survival time compared to the hSyn vector injections. Given these injection differences, and the similar capsid uptake at the site of injection, one would expect more retrograde labeling from the hSyn vector compared to the CAG vector. Clearly, the fact that substantially more retrograde labeling was produced by the CAG vector suggests that parameter differences did not contribute to the observed differences in gene expression levels. Also, given that the hSyn is an endogenous promoter and exhibited substantial gene expression at the site of injection, it appears that promoter silencing likely was not a factor. Therefore, the current data supports our conclusion that the rAAV2-Retro capsid interacted with the hSyn promoter to suppress gene expression in the contralateral FEF and ipsilateral claustrum.

Prior experience suggest that one needs to invoke caution when working in large animal models where experimental conditions are not totally identical. However even in light of differences in injection parameters, the SC data also suggests capsid-promoter interactions. For example, similar to the FEF study, both vectors supported gene expression at the injection site and in the retrogradely labeled corticotectal neurons within the FEF. Although the level of FEF expression was markedly different between the two vectors, this difference could be attributed to the larger injection volume and the longer survival duration for the hSyn vector (Table 5). Importantly, however, the pattern of labeling for both the injection site and the retrograde labeling within the FEF was present for all three cases. In contrast, the hSyn vector also supported robust expression in the retrogradely labeled cerebellotectal neurons while two independent cases where the CAG vector was injected into the superior colliculus failed to show retrograde labeling in the deep cerebellar nuclei, even though CAG driven expression in case 5 was similar to hSyn driven expression in case 6 (FIGS. 19A-19I). These observations provide further evidence supporting the idea that capsid-promoter interactions are taking place across species, capsids, but also along the neuroaxis.

Generally, studies of intraparenchymal AAV injections in the primate CNS have suggested that gene expression in specific neuronal populations depends upon the promoter and, in many instances, promoters only determine the degree of transgene expression. For example, Watakabe et al. (Neurosci. Res. 93:144 (2015)) compared local transduction using AAV1-CaMKII and AAV1-Syn1 to AAV9-CaMKII and AAV9-Syn1 in marmosets. They concluded that the only difference between the two promoters was the degree of transgene expression. In contrast, the present study suggests that capsid-promoter interactions can also affect the pattern of transgene expression, not just the amount of labeling. With the current focus upon generating novel AAV capsids in non-human primates and rodents, the possibility exists that novel transduction properties arise not only from unique capsid binding properties, but also from unknown novel capsid-promoter interactions. Additional experiments will show that this unique observation that we have documented is not restricted to AAV capsid/promoter interactions in the brain, but occurs in peripheral tissue. More importantly, recent data using cell base assays and siRNA library approach have resulted in identification of cellular factors that are linked with AAV vector transcription and in one case bound to AAV capsid (Schreiber et al., CPlos Pathogens 11:e1005082 (2015)). These efforts apparently identify cellular protein candidates that impact all AAV serotypes for gene expression after vector infection and uncoating in the nucleus. The observations we have documented appear to be cell type specific (e.g., neurons vs. oligodendrocytes), promoter specific (CBA vs. CAG), and strongly influenced by subtle amino acid changes in the unique region of minor AAV structural proteins Vp1 and 2 (e.g., AAV9 AU). Therefore, unlike cell based “knock down” assays, future mechanistic studies to further unravel these current observations will require in vivo analysis. In an effort to establish the overall contribution of this novel interaction between the AAV capsid and its genomic cargo, certain attributes of the AAV lifecycle may shed light on these phenomena: namely viral latency. AAV is known to have a bi-phasic lifecycle consisting of lytic when Ad helper is present and latent when absent. Under certain conditions, AAV would be permissive for all steps involved in lytic infection, including receptor binding, trafficking and nuclear entry with viral genomes finally being retained in a conformation not suited for transgene expression. The data derived in both our rodent and primate studies indicate that various AAV capsid/promoter combinations can result in latent genomes, where all steps of virus permissivity take place except the last step of gene expression. Noteworthy, studies by Muzyczka and colleagues (Aydemir et al., J. Virol. 90:7196 (2016)) have identified a region on the AAV capsid that when mutated carryout all steps of infection including uncoating but remain negative for gene expression illustrating precedence for such a phenomenon. Irrespective of mechanism, we document a unique attribute of AAV vectors in both rodent and primate models that until recently remained undescribed: namely capsid/promoter interactions, that dictated cell type transduction profiles regardless of virus permissivity. While these observations may be aligned with aspects of AAV latency, they create potential consternation with respect to influence on AAV viral vectors for human gene therapy, the understanding of which facilitate minimization of possible health risks associated with the transition from animal model to human clinical studies.

TABLE 8
AAV Serotypes and corresponding capsid sequence

| Serotype and where capsid sequence is published | Serotype and where capsid sequence is published |
|---|---|
| AAV3.3b See US20030138772 SEQ ID NO: 72 | AAV3-3 See US20150315612 SEQ ID NO: 200 |
| AAV3-3 See US20150315612 SEQ ID NO: 217 | AAV3a See U.S. Pat. No. 6,156,303 SEQ ID NO: 5 |
| AAV3a See U.S. Pat. No. 6,156,303 | AAV3b See U.S. Pat. No. 6,156,303 SEQ ID NO: 6 |
| SEQ ID NO: 9 | |
| AAV3b See U.S. Pat. No. 6,156,303 | AAV3b See U.S. Pat. No. 6,156,303 SEQ ID NO: 1 |
| SEQ ID NO: 10 | |
| AAV4 See US20140348794 SEQ ID NO: 17 | AAV4 See US20140348794 SEQ ID NO: 5 |
| AAV4 See US20140348794 SEQ ID NO: 3 | AAV4 See US20140348794 SEQ ID NO: 14 |
| AAV4 See US20140348794 SEQ ID NO: 15 | AAV4 See US20140348794 SEQ ID NO: 19 |
| AAV4 See US20140348794 SEQ ID NO: 12 | AAV4 See US20140348794 SEQ ID NO: 13 |
| AAV4 See US20140348794 SEQ ID NO: 7 | AAV4 See US20140348794 SEQ ID NO: 8 |
| AAV4 See US20140348794 SEQ ID NO: 9 | AAV4 See US20140348794 SEQ ID NO: 2 |
| AAV4 See US20140348794 SEQ ID NO: 10 | AAV4 See US20140348794 SEQ ID NO: 11 |
| AAV4 See US20140348794 SEQ ID NO: 18 | AAV4 See US20030138772 SEQ ID NO: 63, |
| | US20160017295 SEQ |
| ID NO: See US20140348794 SEQ ID NO: 4 | AAV4 See US20140348794 SEQ ID NO: 16 |
| AAV4 See US20140348794 SEQ ID NO: 20 | AAV4 See US20140348794 SEQ ID NO: 6 |
| AAV4 See US20140348794 SEQ ID NO: 1 | AAV42.2 See US20030138772 SEQ ID NO: 9 |
| AAV42.2 See US20030138772 SEQ ID NO: 102 | AAV42.3b See US20030138772 SEQ ID NO: 36 |
| AAV42.3B See US20030138772 SEQ ID NO: 107 | AAV42.4 See US20030138772 SEQ ID NO: 33 |
| AAV42.4 See US20030138772 SEQ ID NO: 88 | AAV42.8 See US20030138772 SEQ ID NO: 27 |
| AAV42.8 See US20030138772 SEQ ID NO: 85 | AAV43.1 See US20030138772 SEQ ID NO: 39 |
| AAV43.1 See US20030138772 SEQ ID NO: 92 | AAV43.12 See US20030138772 SEQ ID NO: 41 |
| AAV43.12 See US20030138772 SEQ ID NO: 93 | AAV8 See US20150159173 SEQ ID NO: 15 |
| AAV8 See US20150376240 SEQ ID NO: 7 | AAV8 See US20030138772 SEQ ID NO: 4, |
| | US20150315612 SEQ ID NO: 182 |
| | AAV8 See US20030138772 SEQ ID NO: 95, |
| | US20140359799 SEQ ID NO: 1, |
| | US20150159173 SEQ ID NO: 31, |
| | US20160017295 SEQ ID NO: 8, |
| | U.S. Pat. No. 7,198,951 SEQ ID NO: 7, |
| | US20150315612 SEQ ID NO: 223 |
| AAV8 See US20150376240 SEQ ID NO: 8 | AAV8 See US20150315612 SEQ ID NO: 214 |
| AAV-8b See US20150376240 SEQ ID NO: 5 | AAV-8b See US20150376240 SEQ ID NO: 3 |
| AAV-8h See US20150376240 SEQ ID NO: 6 | AAV-8h See US20150376240 SEQ ID NO: 4 |
| AAV9 See US20030138772 SEQ ID NO: 5 | AAV9 U.S. Pat. No. 7,198,951 SEQ ID NO: 1 |
| AAV9 See US20160017295 SEQ ID NO: 9 | AAV9 See US20030138772 SEQ ID NO: 100, |
| | U.S. Pat. No. 7,198,951 SEQ ID NO: 2 |
| | AAV9 See U.S. Pat. No. 7,198,951 SEQ ID NO: 3 |
| AAV9 (AAVhu.14) See US20150315612 | AAV9 (AAVhu.14) See US20150315612 |
| SEQ ID NO: 3 | SEQ ID NO: 123 |
| AAVA3.1 See US20030138772 SEQ ID NO: 120 | AAVA3.3 See US20030138772 SEQ ID NO: 57 |
| AAVA3.3 See US20030138772 SEQ ID NO: 66 | AAVA3.4 See US20030138772 SEQ ID NO: 54 |
| AAVA3.4 See US20030138772 SEQ ID NO: 68 | AAVA3.5 See US20030138772 SEQ ID NO: 55 |
| AAVA3.5 See US20030138772 SEQ ID NO: 69 | AAVA3.7 See US20030138772 SEQ ID NO: 56 |
| AAVA3.7 See US20030138772 SEQ ID NO: 67 | AAV29. See (AAVbb. 1) 161 US20030138772 |
| | SEQ ID NO: 11 |
| AAVC2 See US20030138772 SEQ ID NO: 61 | AAVCh.5 See US20150159173 SEQ ID NO: 46, |
| | US20150315612 SEQ ID NO: 234 |
| | AAVcy.2 (AAV13.3) See US20030138772 |
| | SEQ ID NO: 15 |
| AAV24.1 See US20030138772 SEQ ID NO: 101 | AAVcy.3 (AAV24.1) See US20030138772 |
| | SEQ ID NO: 16 |
| AAV27.3 See US20030138772 SEQ ID NO: 104 | AAVcy.4 (AAV27.3) See US20030138772 |
| | SEQ ID NO: 17 |
| AAVcy.5 See US20150315612 SEQ ID NO: 227 | AAV7.2 See US20030138772 SEQ ID NO: 103 |
| AAVcy.5 (AAV7.2) See US20030138772 | AAV16.3 See US20030138772 SEQ ID NO: 105 |
| SEQ ID NO: 18 | |
| AAVcy.6 (AAV16.3) See US20030138772 | AAVcy.5 See US20150159173 SEQ ID NO: 8 |
| SEQ ID NO: 10 | |
| AAVcy.5 See US20150159173 SEQ ID NO: 24 | AAVCy.5Rl See US20150159173 |
| AAVCy.5R2 See US20150159173 | AAVCy.5R3 See US20150159173 |
| AAVCy.5R4 See US20150159173 | AAVDJ See US20140359799 SEQ ID NO: 3, |
| | U.S. Pat. No. 7,588,772 SEQ ID NO: 2 |
| | AAVDJ See US20140359799 SEQ ID NO: 2, |
| | U.S. Pat. No. 7,588,772 SEQ ID NO: 1 |
| | AAVDJ-8 See U.S. Pat. No. 7,588,772; |
| | Grimm et al 2008 |
| AAVDJ-8 See U.S. Pat. No. 7,588,772; | AAVF5 See US20030138772 SEQ ID NO: 110 |
| Grimm et al 2008 | |
| AAVH2 See US20030138772 SEQ ID NO: 26 | AAVH6 See US20030138772 SEQ ID NO: 25 |
| AAVhEl. l See U.S. Pat. No. 9,233,131 | AAVhErl.14 See U.S. Pat. No. 9,233,131 |
| SEQ ID NO: 44 | SEQ ID NO: 46 |
| AAVhErl.16 See U.S. Pat. No. 9,233,131 | AAVhErl.18 See U.S. Pat. No. 9,233,131 |
| SEQ ID NO: 48 | SEQ ID NO: 49 |
| AAVhErl.23 (AAVhEr2.29) See | AAVhErl.35 See U.S. Pat. No. 9,233,131 |
| U.S. Pat. No. 9,233,131 SEQ ID NO: 53 | SEQ ID NO: 50 |
| AAVhErl.36 See U.S. Pat. No. 9,233,131 | AAVhErl.5 See U.S. Pat. No. 9,233,131 |
| SEQ ID NO: 52 | SEQ ID NO: 45 |
| AAVhErl.7 See U.S. Pat. No. 9,233,131 | AAVhErl.8 See U.S. Pat. No. 9,233,131 |
| SEQ ID NO: 51 | SEQ ID NO: 47 |
| AAVhEr2.16 See U.S. Pat. No. 9,233,131 | AAVhEr2.30 See U.S. Pat. No. 9,233,131 |
| SEQ ID NO: 55 | SEQ ID NO: 56 |
| AAVhEr2.31 See U.S. Pat. No. 9,233,131 | AAVhEr2.36 See U.S. Pat. No. 9,233,131 |
| SEQ ID NO: 58 | SEQ ID NO: 57 |
| AAVhEr2.4 See U.S. Pat. No. 9,233,131 | AAVhEr3.1 See U.S. Pat. No. 9,233,131 |
| SEQ ID NO: 54 | SEQ ID NO: 59 |
| AAVhu.l See US20150315612 SEQ ID NO: 46 | AAVhu.l See US20150315612 SEQ ID NO: 144 |
| AAVhu.lO (AAV16.8) See US20150315612 | AAVhu.lO (AAV16.8) See US20150315612 |
| SEQ ID NO: 56 | SEQ ID NO: 156 |
| AAVhu.l l (AAV16.12) See | AAVhu.l l (AAV16.12) See US20150315612 |
| US20150315612 SEQ ID NO: 57 | SEQ ID NO: 153 |
| AAVhu.12 See US20150315612 SEQ ID NO: 59 | AAVhu.12 See US20150315612 SEQ ID NO: 154 |
| AAVhu.13 See US20150159173 SEQ ID NO: 16, | |
| US20150315612 SEQ ID NO: 71 | |
| AAVhu.13 See US20150159173 SEQ ID NO: 32, | |
| US20150315612 SEQ ID NO: 129 | |
| AAVhu.136.1 See US20150315612 | AAVhu.140.1 See US20150315612 |
| SEQ ID NO 165 | SEQ ID NO 166 |
| AAVhu.140.2 See US20150315612 | AAVhu.145.6 See US20150315612 SEQ ID No: 178 |
| SEQ ID NO 167 | |
| AAVhu.15 See US20150315612 SEQ ID NO: 147 | AAVhu.15 (AAV33.4) See US20150315612 |
| | SEQ ID NO: 50 |
| AAVhu.156.1 See US20150315612 | AAVhu.16 See US20150315612 SEQ ID NO 148 |
| SEQ ID No: 179 | |
| AAVhu.l6 (AAV33.8) See US20150315612 | AAVhu.17 See US20150315612 SEQ ID NO 83 |
| SEQ ID NO 51 | |
| AAVhu.l7 (AAV33.12) See | AAVhu.172.1 See US20150315612 SEQ ID NO 171 |
| US20150315612 SEQ ID NO 4 | |
| AAVhu.172.2 See US20150315612 | AAVhu.173.4 See US20150315612 SEQ ID NO 173 |
| SEQ ID NO 172 | |
| AAVhu.173.8 See US20150315612 | AAVhu.18 See US20150315612 SEQ ID NO 52 |
| SEQ ID NO 175 | |
| AAVhu.18 See US20150315612 SEQ ID NO 149 | AAVhu.19 See US20150315612 SEQ ID NO 62 |
| AAVhu.19 See US20150315612 SEQ ID NO 133 | AAVhu.2 See US20150315612 SEQ ID NO 48 |
| AAVhu.2 See US20150315612 SEQ ID NO 143 | AAVhu.20 See US20150315612 SEQ ID NO 63 |
| AAVhu.20 See US20150315612 SEQ ID NO 134 | AAVhu.21 See US20150315612 SEQ ID NO 65 |
| AAVhu.21 See US20150315612 SEQ ID NO 135 | AAVhu.22 See US20150315612 SEQ ID NO 67 |
| AAVhu.22 239 US20150315612 SEQ ID NO 138 | AAVhu.23 See US20150315612 SEQ ID NO 60 |
| AAVhu.23.2 See US20150315612 SEQ ID NO 137 | AAVhu.24 See US20150315612 SEQ ID NO 66 |
| AAVhu.24 See US20150315612 SEQ ID NO 136 | AAVhu.25 See US20150315612 SEQ ID NO 49 |
| AAVhu.25 See US20150315612 SEQ ID NO 146 | AAVhu.26 See US20150159173 SEQ ID NO 17, |
| | US20150315612 SEQ ID NO: 61 |
| | AAVhu.26 See US20150159173 SEQ ID NO: 33, |
| | US20150315612 SEQ |
| | AAVhu.27 See US20150315612 SEQ ID NO: 64 |
| AAVhu.27 See US20150315612 SEQ ID NO: 140 | AAVhu.28 See US20150315612 SEQ ID NO: 68 |
| AAVhu.28 See US20150315612 SEQ ID NO: 130 | AAVhu.29 See US20150315612 SEQ ID NO: 69 |
| AAVhu.29 See US20150159173 SEQ ID NO: 42, | |
| US20150315612 SEQ ID NO: 132 | |
| AAVhu.29 See US20150315612 SEQ ID NO: 225 | AAVhu.29R See US20150159173 |
| AAVhu.3 See US20150315612 SEQ ID NO: 44 | AAVhu.3 See US20150315612 SEQ ID NO: 145 |
| AAVhu.30 See US20150315612 SEQ ID NO: 70 | AAVhu.30 See US20150315612 SEQ ID NO: 131 |
| AAVhu.31 See US20150315612 SEQ ID NO: 1 | AAVhu.31 See US20150315612 SEQ ID NO: 121 |
| AAVhu.32 See US20150315612 SEQ ID NO: 2 | AAVhu.32 See US20150315612 SEQ ID NO: 122 |
| AAVhu.33 See US20150315612 SEQ ID NO: 75 | AAVhu.33 See US20150315612 SEQ ID NO: 124 |
| AAVhu.34 See US20150315612 SEQ ID NO: 72 | AAVhu.34 See US20150315612 SEQ ID NO: 125 |
| AAVhu.35 See US20150315612 SEQ ID NO: 73 | AAVhu.35 See US20150315612 SEQ ID NO: 164 |
| AAVhu.36 See US20150315612 SEQ ID NO: 74 | AAVhu.36 See US20150315612 SEQ ID NO: 126 |
| AAVhu.37 See US20150159173 SEQ ID NO: 34, | |
| US20150315612 SEQ ID NO: 88 | |
| AAVhu.37 (AAV106.1) See | |
| US20150315612 SEQ ID NO: 10, | |
| US20150159173 SEQ ID NO: 18 | |
| AAVhu.38 See US20150315612 SEQ ID NO 161 | AAVhu.39 See US20150315612 SEQ ID NO 102 |
| AAVhu.39 (AAVLG-9) See | AAVhu.4 See US20150315612 SEQ ID NO 47 |
| US20150315612 SEQ ID NO 24 | |
| AAVhu.4 See US20150315612 SEQ ID NO 141 | AAVhu.40 See US20150315612 SEQ ID NO 87 |
| AAVhu.40 (AAV114.3) See | AAVhu.41 See US20150315612 SEQ ID NO: 91 |
| US20150315612 SEQ ID No: 11 | |
| AAVhu.41 (AAV127.2) See | AAVhu.42 See US20150315612 SEQ ID NO: 85 |
| US20150315612 SEQ ID NO: 6 | |
| AAVhu.42 (AAV127.5) See | AAVhu.43 See US20150315612 SEQ ID NO: 160 |
| US20150315612 SEQ ID NO: 8 | |
| AAVhu.43 See US20150315612 SEQ ID NO: 236 | AAVhu.43 (AAV128.1) See US20150315612 |
| | SEQ ID NO: 80 |
| AAVhu.44 See US20150159173 SEQ ID NO: 45, | |
| US20150315612 SEQ ID NO: 158 | |
| AAVhu.44 (AAV128.3) See | AAVhu.44Rl See US20150159173 |
| US20150315612 SEQ ID NO: 81 | |
| AAVhu.44R2 See US20150159173 | AAVhu.44R3 See US20150159173 |
| AAVhu.45 See US20150315612 SEQ ID NO: 76 | AAVhu.45 See US20150315612 SEQ ID NO: 127 |
| AAVhu.46 See US20150315612 SEQ ID NO: 82 | AAVhu.46 See US20150315612 SEQ ID NO: 159 |
| AAVhu.46 See US20150315612 SEQ ID NO: 224 | AAVhu.47 See US20150315612 SEQ ID NO: 77 |
| AAVhu.47 See US20150315612 SEQ ID NO: 128 | AAVhu.48 See US20150159173 SEQ ID NO: 38 |
| AAVhu.48 See US20150315612 SEQ ID NO: 157 | AAVhu.48 (AAV130.4) See US20150315612 |
| | SEQ ID NO: 78 |
| AAVhu.48Rl See US20150159173 | AAVhu.48R2 See US20150159173 |
| AAVhu.48R3 See US20150159173 | AAVhu.49 See US20150315612 SEQ ID NO 209 |
| AAVhu.49 See US20150315612 SEQ ID NO 189 | AAVhu.5 See US20150315612 SEQ ID NO 45 |
| AAVhu.5 See US20150315612 SEQ ID NO 142 | AAVhu.51 See US20150315612 SEQ ID NO 208 |
| AAVhu.51 See US20150315612 SEQ ID NO 190 | AAVhu.52 See US20150315612 SEQ ID NO 210 |
| AAVhu.52 See US20150315612 SEQ ID NO 191 | AAVhu.53 See US20150159173 SEQ ID NO 19 |
| AAVhu.53 See US20150159173 SEQ ID NO 35 | AAVhu.53 (AAV145.1) See US20150315612 |
| | SEQ ID NO 176 |
| AAVhu.54 See US20150315612 SEQ ID NO 188 | AAVhu.54 (AAV145.5) See US20150315612 |
| | SEQ ID No: 177 |
| AAVhu.55 See US20150315612 SEQ ID NO 187 | AAVhu.56 See US20150315612 SEQ ID NO 205 |
| AAVhu.56 (AAV145.6) See | AAVhu.56 (AAV145.6) See US20150315612 |
| US20150315612 SEQ ID NO 168 | SEQ ID NO 192 |
| AAVhu.57 See US20150315612 SEQ ID NO 206 | AAVhu.57 See US20150315612 SEQ ID NO 169 |
| AAVhu.57 See US20150315612 SEQ ID NO 193 | AAVhu.58 See US20150315612 SEQ ID NO 207 |
| AAVhu.58 See US20150315612 SEQ ID NO 194 | AAVhu.6 (AAV3.1) See US20150315612 |
| | SEQ ID NO: 5 |
| AAVhu.6 (AAV3.1) See US20150315612 | AAVhu.60 See US20150315612 SEQ ID NO: 184 |
| SEQ ID NO: 84 | |
| AAVhu.60 (AAV161.10) See | AAVhu.61 See US20150315612 SEQ ID NO: 185 |
| US20150315612 SEQ ID NO: 170 | |
| AAVhu.61 (AAV161.6) See | AAVhu.63 See US20150315612 SEQ ID NO: 204 |
| US20150315612 SEQ ID NO: 174 | |
| AAVhu.63 See US20150315612 SEQ ID NO: 195 | AAVhu.64 See US20150315612 SEQ ID NO: 212 |
| AAVhu.64 See US20150315612 SEQ ID NO: 196 | AAVhu.66 See US20150315612 SEQ ID NO: 197 |
| AAVhu.67 See US20150315612 SEQ ID NO: 215 | AAVhu.67 See US20150315612 SEQ ID NO: 198 |
| AAVhu.7 See US20150315612 SEQ ID NO: 226 | AAVhu.7 See US20150315612 SEQ ID NO: 150 |
| AAVhu.7 (AAV7.3) See US20150315612 | AAVhu.71 See US20150315612 SEQ ID NO: 79 |
| SEQ ID NO: 55 | |
| AAVhu.8 See US20150315612 SEQ ID NO: 53 | AAVhu.8 See US20150315612 SEQ ID NO: 12 |
| AAVhu.8 See US20150315612 SEQ ID NO: 151 | AAVhu.9 (AAV3.1) See US20150315612 |
| | SEQ ID NO: 58 |
| AAVhu.9 (AAV3.1) See US20150315612 | AAV-LK01 See US20150376607 SEQ ID NO: 2 |
| SEQ ID NO: 155 | |
| AAV-LK01 See US20150376607 SEQ ID NO: 29 | AAV-LK02 See US20150376607 SEQ ID NO: 3 |
| AAV-LK02 See US20150376607 SEQ ID NO: 30 | AAV-LK03 See US20150376607 SEQ ID NO: 4 |
| AAV-LK03 See WO2015121501 SEQ ID NO: 12, | |
| US20150376607 SEQ ID NO: 31 | |
| AAV-LK04 See US20150376607 SEQ ID NO: 5 | AAV-LK04 See US20150376607 SEQ ID NO: 32 |
| AAV-LK05 See US20150376607 SEQ ID NO: 6 | AAV-LK05 See US20150376607 SEQ ID NO: 33 |
| AAV-LK06 See US20150376607 SEQ ID NO: 7 | AAV-LK06 See US20150376607 SEQ ID NO: 34 |
| AAV-LK07 See US20150376607 SEQ ID NO: 8 | AAV-LK07 See US20150376607 SEQ ID NO: 35 |
| AAV-LK08 See US20150376607 SEQ ID NO: 9 | AAV-LK08 See US20150376607 SEQ ID NO: 36 |
| AAV-LK09 See US20150376607 SEQ ID NO: 10 | AAV-LK09 See US20150376607 SEQ ID NO: 37 |
| AAV-LK10 See US20150376607 SEQ ID NO: 11 | AAV-LK10 See US20150376607 SEQ ID NO: 38 |
| AAV-LK11 See US20150376607 SEQ ID NO: 12 | AAV-LK11 See US20150376607 SEQ ID NO: 39 |
| AAV-LK12 See US20150376607 SEQ ID NO: 13 | AAV-LK12 See US20150376607 SEQ ID NO: 40 |
| AAV-LK13 See US20150376607 SEQ ID NO: 14 | AAV-LK13 See US20150376607 SEQ ID NO: 41 |
| AAV-LK14 See US20150376607 SEQ ID NO: 15 | AAV-LK14 See US20150376607 SEQ ID NO: 42 |
| AAV-LK15 See US20150376607 SEQ ID NO: 16 | AAV-LK15 See US20150376607 SEQ ID NO: 43 |
| AAV-LK16 See US20150376607 SEQ ID NO: 17 | AAV-LK16 See US20150376607 SEQ ID NO: 44 |
| AAV-LK17 See US20150376607 SEQ ID NO: 18 | AAV-LK17 See US20150376607 SEQ ID NO: 45 |
| AAV-LK18 See US20150376607 SEQ ID NO: 19 | AAV-LK18 See US20150376607 SEQ ID NO: 46 |
| AAV-LK19 See US20150376607 SEQ ID NO: 20 | AAV-LK19 See US20150376607 SEQ ID NO: 47 |
| AAV-PAEC See US20150376607 SEQ ID NO: 1 | AAV-PAEC See US20150376607 SEQ ID NO: 48 |
| AAV-PAEC11 See US20150376607 SEQ ID NO: 26 | AAV-PAEC11 See US20150376607 SEQ ID NO: 54 |
| AAV-PAEC 12 See US20150376607 | AAV-PAEC 12 See US20150376607 SEQ ID NO: 51 |
| SEQ ID NO: 27 | |
| AAV-PAEC 13 See US20150376607 | AAV-PAEC 13 See US20150376607 SEQ ID NO: 49 |
| SEQ ID NO: 28 | |
| AAV-PAEC2 See US20150376607 SEQ ID NO: 21 | AAV-PAEC2 See US20150376607 SEQ ID NO: 56 |
| AAV-PAEC4 See US20150376607 SEQ ID NO: 22 | AAV-PAEC4 See US20150376607 SEQ ID NO: 55 |
| AAV-PAEC6 See US20150376607 SEQ ID NO: 23 | AAV-PAEC6 See US20150376607 SEQ ID NO: 52 |
| AAV-PAEC7 See US20150376607 SEQ ID NO: 24 | AAV-PAEC7 See US20150376607 SEQ ID NO: 53 |
| AAV-PAEC8 See US20150376607 SEQ ID NO: 25 | AAV-PAEC8 See US20150376607 SEQ ID NO: 50 |
| AAVpi.l See US20150315612 SEQ ID NO: 28 | AAVpi.l See US20150315612 SEQ ID NO: 93 |
| | AAVpi.2 408 US20150315612 SEQ ID NO: 30 |
| AAVpi.2 See US20150315612 SEQ ID NO: 95 | AAVpi.3 See US20150315612 SEQ ID NO: 29 |
| AAVpi.3 See US20150315612 SEQ ID NO: 94 | AAVrh.10 See US20150159173 SEQ ID NO: 9 |
| AAVrh.10 See US20150159173 SEQ ID NO: 25 | AAV44.2 See US20030138772 SEQ ID NO: 59 |
| AAVrh.10 (AAV44.2) See US20030138772 | AAV42.1B See US20030138772 SEQ ID NO: 90 |
| SEQ ID NO: 81 | |
| AAVrh.l2 (AAV42.1b) See | AAVrh.13 See US20150159173 SEQ ID NO: 10 |
| US20030138772 SEQ ID NO: 30 | |
| AAVrh.13 See US20150159173 SEQ ID NO: 26 | AAVrh.13 See US20150315612 SEQ ID NO: 228 |
| AAVrh.l3R See US20150159173 | AAV42.3A See US20030138772 SEQ ID NO: 87 |
| AAVrh.l4 (AAV42.3a) See | AAV42.5A See US20030138772 SEQ ID NO: 89 |
| US20030138772 SEQ ID NO: 32 | |
| AAVrh.l7 (AAV42.5a) See | AAV42.5B See US20030138772 SEQ ID NO: 91 |
| US20030138772 SEQ ID NO: 34 | |
| AAVrh.l8 (AAV42.5b) See | AAV42.6B See US20030138772 SEQ ID NO: 112 |
| US20030138772 SEQ ID NO: 29 | |
| AAVrh.l9 (AAV42.6b) See | AAVrh.2 See US20150159173 SEQ ID NO: 39 |
| US20030138772 SEQ ID NO: 38 | |
| AAVrh.2 See US20150315612 SEQ ID NO: 231 | AAVrh.20 See US20150159173 SEQ ID NO: 1 |
| AAV42.10 See US20030138772 SEQ ID NO: 106 | AAVrh.21 (AAV42.10) See US20030138772 |
| | SEQ ID NO: 35 |
| AAV42.11 See US20030138772 SEQ ID NO: 108 | AAVrh.22 (AAV42.11) See US20030138772 |
| | SEQ ID NO: 37 |
| AAV42.12 See US20030138772 SEQ ID NO: 113 | AAVrh.23 (AAV42.12) See US20030138772 |
| | SEQ ID NO: 58 |
| AAV42.13 See US20030138772 SEQ ID NO: 86 | AAVrh.24 (AAV42.13) See US20030138772 |
| | SEQ ID NO: 31 |
| AAV42.15 See US20030138772 SEQ ID NO: 84 | AAVrh.25 (AAV42.15) See US20030138772 |
| | SEQ ID NO: 28 |
| AAVrh.2R See US20150159173 | AAVrh.31 (AAV223.1) See US20030138772 |
| | SEQ ID NO: 48 |
| AAVC1 See US20030138772 SEQ ID NO: 60 | AAVrh.32 (AAVC1) See 446 US20030138772 |
| | SEQ ID NO: 19 |
| AAVrh.32/33 See US20150159173 SEQ ID NO: 2 | AAVrh.51 (AAV2-5) See US20150315612 |
| | SEQ ID NO: 104 |
| AAVrh.52 (AAV3-9) See US20150315612 | AAVrh.52 (AAV3-9) See US20150315612 |
| SEQ ID NO: 18 | SEQ ID NO: 96 |
| AAVrh.53 See US20150315612 SEQ ID NO: 97 | AAVrh.53 (AAV3-11) See US20150315612 |
| | SEQ ID NO: 17 |
| AAVrh.53 (AAV3-11) See US20150315612 | AAVrh.54 See US20150315612 SEQ ID NO: 40 |
| SEQ ID NO: 186 | |
| AAVrh.54 See US20150159173 SEQ ID NO: 49, | |
| US20150315612 SEQ ID NO: 116 | |
| AAVrh.55 See US20150315612 SEQ ID NO: 37 | AAVrh.55 (AAV4-19) See US20150315612 |
| | SEQ ID NO: 117 |
| AAVrh.56 v US20150315612 SEQ ID NO: 54 | AAVrh.56 See US20150315612 SEQ ID NO: 152 |
| AAVrh.57 See 497 US20150315612 | AAVrh.57 See US20150315612 SEQ ID NO: 105 |
| SEQ ID NO: 26 | |
| AAVrh.58 See US20150315612 SEQ ID NO: 27 | AAVrh.58 See US20150159173 SEQ ID NO: 48, |
| | US20150315612 SEQ ID NO: 106 |
| | AAVrh.58 See US20150315612 SEQ ID NO: 232 |
| AAVrh.59 See US20150315612 SEQ ID NO: 42 | AAVrh.59 See US20150315612 SEQ ID NO: 110 |
| AAVrh.60 See US20150315612 SEQ ID NO: 31 | AAVrh.60 See US20150315612 SEQ ID NO: 120 |
| AAVrh.61 See US20150315612 SEQ ID NO: 107 | AAVrh.61 (AAV2-3) See US20150315612 |
| | SEQ ID NO: 21 |
| AAVrh.62 (AAV2-15) See US20150315612 | AAVrh.62 (AAV2-15) See US20150315612 |
| SEQ ID No: 33 | SEQ ID NO: 114 |
| AAVrh.64 See US20150315612 SEQ ID No: 15 | AAVrh.64 See US20150159173 SEQ ID NO: 43, |
| | US20150315612 SEQ ID NO: 99 |
| | AAVrh.64 See US20150315612 SEQ ID NO: 233 |
| AAVRh.64Rl See US20150159173 | AAVRh.64R2 See US20150159173 |
| AAVrh.65 See US20150315612 SEQ ID NO: 35 | AAVrh.65 See US20150315612 SEQ ID NO: 112 |
| AAVrh.67 See US20150315612 SEQ ID NO: 36 | AAVrh.67 See US20150315612 SEQ ID NO: 230 |
| AAVrh.67 See US20150159173 SEQ ID NO: 47, | |
| US20150315612 SEQ ID NO: 113 | |
| AAVrh.68 See US20150315612 SEQ ID NO: 16 | AAVrh.68 See US20150315612 SEQ ID NO: 100 |
| AAVrh.69 See US20150315612 SEQ ID NO: 39 | AAVrh.69 See US20150315612 SEQ ID NO: 119 |
| AAVrh.70 See US20150315612 SEQ ID NO: 20 | AAVrh.70 See US20150315612 SEQ ID NO: 98 |
| AAVrh.71 See US20150315612 SEQ ID NO: 162 | AAVrh.72 See US20150315612 SEQ ID NO: 9 |
| AAVrh.73 See US20150159173 SEQ ID NO: 5 | AAVrh.74 See US20150159173 SEQ ID NO: 6 |
| AAVrh.8 See US20150159173 SEQ ID NO: 41 | AAVrh.8 See US20150315612 SEQ ID NO: 235 |
| AAVrh.8R See US20150159173, | AAVrh.8R A586R mutant See WO2015168666 |
| WO2015168666 SEQ ID NO: 9 | SEQ ID NO: 10 |
| AAVrh.8R R533A mutant See | BAAV (bovine AAV) See U.S. Pat. No. 9,193,769 |
| WO2015168666 SEQ ID NO: 11 | SEQ ID NO: 8 |
| BAAV (bovine AAV) See | BAAV (bovine AAV) See U.S. Pat. No. 9,193,769 |
| U.S. Pat. No. 9,193,769 SEQ ID NO: 10 | SEQ ID NO: 4 |
| BAAV (bovine AAV) See | BAAV (bovine AAV) See U.S. Pat. No. 9,193,769 |
| U.S. Pat. No. 9,193,769 SEQ ID NO: 2 | SEQ ID NO: 6 |
| BAAV (bovine AAV) See | BAAV (bovine AAV) See U.S. Pat. No. 9,193,769 |
| U.S. Pat. No. 9,193,769 SEQ ID NO: 1 | SEQ ID NO: 5 |
| BAAV (bovine AAV) See | BAAV (bovine AAV) See U.S. Pat. No. 9,193,769 |
| U.S. Pat. No. 9,193,769 SEQ ID NO: 3 | SEQ ID NO: 11 |
| BAAV (bovine AAV) See | BAAV (bovine AAV) See U.S. Pat. No. 7,427,396 |
| U.S. Pat. No. 7,427,396 SEQ ID NO: 5 | SEQ ID NO: 6 |
| BAAV (bovine AAV) See | BAAV (bovine AAV) See U.S. Pat. No. 9,193,769 |
| U.S. Pat. No. 9,193,769 SEQ ID NO: 7 | SEQ ID NO: 9 |
| BNP61 AAV See US20150238550 SEQ ID NO: 1 | BNP61 AAV See US20150238550 SEQ ID NO: 2 |
| BNP62 AAV See US20150238550 SEQ ID NO: 3 | BNP63 AAV See US20150238550 SEQ ID NO: 4 |
| caprine AAV See U.S. Pat. No. 7,427,396 | caprine AAV See U.S. Pat. No. 7,427,396 |
| SEQ ID NO: 3 | SEQ ID NO: 4 |
| true type AAV (ttAAV) See | AAAV (Avian AAV) See U.S. Pat. No. 9,238,800 |
| WO2015121501 SEQ ID NO: 2 | SEQ ID NO: 12 |
| AAAV (Avian AAV) See | AAAV (Avian AAV) See U.S. Pat. No. 9,238,800 |
| U.S. Pat. No. 9,238,800 SEQ ID NO: 2 | SEQ ID NO: 6 |
| AAAV (Avian AAV) See | AAAV (Avian AAV) See U.S. Pat. No. 9,238,800 |
| U.S. Pat. No. 9,238,800 SEQ ID NO: 4 | SEQ ID NO: 8 |
| AAAV (Avian AAV) See | AAAV (Avian AAV) See U.S. Pat. No. 9,238,800 |
| U.S. Pat. No. 9,238,800 SEQ ID NO: 14 | SEQ ID NO: 10 |
| AAAV (Avian AAV) See | AAAV (Avian AAV) See U.S. Pat. No. 9,238,800 |
| U.S. Pat. No. 9,238,800 SEQ ID NO: 15 | SEQ ID NO: 5 |
| AAAV (Avian AAV) See | AAAV (Avian AAV) See U.S. Pat. No. 9,238,800 |
| U.S. Pat. No. 9,238,800 SEQ ID NO: 9 | SEQ ID NO: 3 |
| AAAV (Avian AAV) See | AAAV (Avian AAV) See U.S. Pat. No. 9,238,800 |
| U.S. Pat. No. 9,238,800 SEQ ID NO: 7 | SEQ ID NO: 11 |
| AAAV (Avian AAV) See | AAAV (Avian AAV) See U.S. Pat. No. 9,238,800 |
| U.S. Pat. No. 9,238,800 SEQ ID NO: 13 | SEQ ID NO: 1 |
| AAV Shuffle 100-1 See US20160017295 | AAV Shuffle 100-1 See US20160017295 |
| SEQ ID NO: 23 | SEQ ID NO: 11 |
| AAV Shuffle 100-2 See US20160017295 | AAV Shuffle 100-2 See US20160017295 |
| SEQ ID NO: 37 | SEQ ID NO: 29 |
| AAV Shuffle 100-3 See US20160017295 | AAV Shuffle 100-3 See US20160017295 |
| SEQ ID NO: 24 | SEQ ID NO: 12 |
| AAV Shuffle 100-7 See US20160017295 | AAV Shuffle 100-7 See US20160017295 |
| SEQ ID NO: 25 | SEQ ID NO: 13 |
| AAV Shuffle 10-2 See US20160017295 | AAV Shuffle 10-2 See US20160017295 |
| SEQ ID NO: 34 | SEQ ID NO: 26 |
| AAV Shuffle 10-6 See US20160017295 | AAV Shuffle 10-6 See US20160017295 |
| SEQ ID NO: 35 | SEQ ID NO: 27 |
| AAV Shuffle 10-8 See US20160017295 | AAV Shuffle 10-8 See US20160017295 |
| SEQ ID NO: 36 | SEQ ID NO: 28 |
| AAV SM 100-10 See US20160017295 | AAV SM 100-10 See US20160017295 |
| SEQ ID NO: 41 | SEQ ID NO: 33 |
| AAV SM 100-3 See US20160017295 | AAV SM 100-3 See US20160017295 |
| SEQ ID NO: 40 | SEQ ID NO: 32 |
| AAV SM 10-1 See US20160017295 | AAV SM 10-1 See US20160017295 SEQ ID NO: 30 |
| SEQ ID NO: 38 | |
| AAV SM 10-2 See US20160017295 | AAV SM 10-2 See US20160017295 SEQ ID NO: 22 |
| SEQ ID NO: 10 | |
| AAV SM 10-8 See US20160017295 | AAV SM 10-8 See US20160017295 SEQ ID NO: 31 |
| SEQ ID NO: 39 | |
| AAV CBr-7.1 See WO2016065001 SEQ ID NO: 4 | AAV CBr-7.1 See WO2016065001 SEQ ID NO: 54 |
| AAV CBr-7.10 See WO2016065001 SEQ ID NO: 11 | AAV CBr-7.10 See WO2016065001 SEQ ID NO: 61 |
| AAV CBr-7.2 See WO2016065001 SEQ ID NO: 5 | AAV CBr-7.2 See WO2016065001 SEQ ID NO: 55 |
| AAV CBr-7.3 See WO2016065001 SEQ ID NO: 6 | AAV CBr-7.3 See WO2016065001 SEQ ID NO: 56 |
| AAV CBr-7.4 See WO2016065001 SEQ ID NO: 7 | AAV CBr-7.4 See WO2016065001 SEQ ID NO: 57 |
| AAV CBr-7.5 See WO2016065001 SEQ ID NO: 8 | AAV CHt-6.6 See WO2016065001 SEQ ID NO: 35 |
| AAV CHt-6.6 See WO2016065001 SEQ ID NO: 85 | AAV CHt-6.7 See WO2016065001 SEQ ID NO: 36 |
| AAV CHt-6.7 See WO2016065001 SEQ ID NO: 86 | AAV CHt-6.8 See WO2016065001 SEQ ID NO: 37 |
| AAV CHt-6.8 See WO2016065001 SEQ ID NO: 87 | AAV CHt-Pl See WO2016065001 SEQ ID NO: 29 |
| AAV CHt-Pl See WO2016065001 SEQ ID NO: 79 | AAV CHt-P2 See WO2016065001 SEQ ID NO: 1 |
| AAV CHt-P2 See WO2016065001 SEQ ID NO: 51 | AAV CHt-P5 See WO2016065001 SEQ ID NO: 2 |
| AAV CHt-P5 See WO2016065001 SEQ ID NO: 52 | AAV CHt-P6 See WO2016065001 SEQ ID NO: 30 |
| AAV CHt-P6 See WO2016065001 SEQ ID NO: 80 | AAV CHt-P8 See WO2016065001 SEQ ID NO: 31 |
| AAV CHt-P8 See WO2016065001 SEQ ID NO: 81 | AAV CHt-P9 See WO2016065001 SEQ ID NO: 3 |
| AAV CHt-P9 See WO2016065001 SEQ ID NO: 53 | AAV CKd-1 See U.S. Pat. No. 8,734,809 |
| | SEQ ID NO 57 |
| AAV CKd-1 See U.S. Pat. No. 8,734,809 | AAV CKd-10 See U.S. Pat. No. 8,734,809 |
| SEQ ID NO 131 | SEQ ID NO 58 |
| AAV CKd-10 See U.S. Pat. No. 8,734,809 | AAV CKd-2 See U.S. Pat. No. 8,734,809 |
| SEQ ID NO 132 | SEQ ID NO 59 |
| AAV CKd-2 See U.S. Pat. No. 8,734,809 | AAV CKd-3 See U.S. Pat. No. 8,734,809 |
| SEQ ID NO 133 | SEQ ID NO 60 |
| AAV CKd-3 See U.S. Pat. No. 8,734,809 | AAV CKd-4 See U.S. Pat. No. 8,734,809 |
| SEQ ID NO 134 | SEQ ID NO 61 |
| AAV CKd-4 See U.S. Pat. No. 8,734,809 | AAV CKd-6 See U.S. Pat. No. 8,734,809 |
| SEQ ID NO 135 | SEQ ID NO 62 |
| AAV CKd-6 See U.S. Pat. No. 8,734,809 | AAV CKd-7 See U.S. Pat. No. 8,734,809 |
| SEQ ID NO 136 | SEQ ID NO 63 |
| AAV CKd-7 See U.S. Pat. No. 8,734,809 | AAV CKd-8 See U.S. Pat. No. 8,734,809 |
| SEQ ID NO 137 | SEQ ID NO 64 |
| AAV CKd-8 See U.S. Pat. No. 8,734,809 | AAV CKd-B 1 See U.S. Pat. No. 8,734,809 |
| SEQ ID NO 138 | SEQ ID NO 73 |
| AAV CKd-B 1 See U.S. Pat. No. 8,734,809 | AAV CKd-B2 See U.S. Pat. No. 8,734,809 |
| SEQ ID NO 147 | SEQ ID NO 74 |
| AAV CKd-B2 See U.S. Pat. No. 8,734,809 | AAV CKd-B3 See U.S. Pat. No. 8,734,809 |
| SEQ ID NO 148 | SEQ ID NO 75 |
| AAV CKd-B3 See U.S. Pat. No. 8,734,809 | AAV CKd-B3 See U.S. Pat. No. 8,734,809 |
| | SEQ ID NO 149 |
| AAV CLv-1 See U.S. Pat. No. 8,734,809 | AAV CLv-1 See U.S. Pat. No. 8,734,809 |
| SEQ ID NO: 65 | SEQ ID NO: 139 |
| AAV CLvl-1 See U.S. Pat. No. 8,734,809 | AAV Civ 1-10 See U.S. Pat. No. 8,734,809 |
| SEQ ID NO: 171 | SEQ ID NO: 178 |
| AAV CLvl-2 See U.S. Pat. No. 8,734,809 | AAV CLv-12 See U.S. Pat. No. 8,734,809 |
| SEQ ID NO: 172 | SEQ ID NO: 66 |
| AAV CLv-12 See U.S. Pat. No. 8,734,809 | AAV CLvl-3 See U.S. Pat. No. 8,734,809 |
| SEQ ID NO: 140 | SEQ ID NO: 173 |
| AAV CLv-13 See U.S. Pat. No. 8,734,809 | AAV CLv-13 See U.S. Pat. No. 8,734,809 |
| SEQ ID NO: 67 | SEQ ID NO: 141 |
| AAV CLvl-4 See U.S. Pat. No. 8,734,809 | AAV Civ 1-7 See U.S. Pat. No. 8,734,809 |
| SEQ ID NO: 174 | SEQ ID NO: 175 |
| AAV Civ 1-8 See U.S. Pat. No. 8,734,809 | AAV Civ 1-9 See U.S. Pat. No. 8,734,809 |
| SEQ ID NO: 176 | SEQ ID NO: 177 |
| AAV CLv-2 See U.S. Pat. No. 8,734,809 | AAV CLv-2 See U.S. Pat. No. 8,734,809 |
| SEQ ID NO: 68 | SEQ ID NO: 142 |
| AAV CLv-3 See U.S. Pat. No. 8,734,809 | AAV CLv-3 See U.S. Pat. No. 8,734,809 |
| SEQ ID NO: 69 | SEQ ID NO: 143 |
| AAV CLv-4 See U.S. Pat. No. 8,734,809 | AAV CLv-4 See U.S. Pat. No. 8,734,809 |
| SEQ ID NO: 70 | SEQ ID NO: 144 |
| AAV CLv-6 See U.S. Pat. No. 8,734,809 | AAV CLv-6 See U.S. Pat. No. 8,734,809 |
| SEQ ID NO: 71 | SEQ ID NO: 145 |
| AAV CLv-8 See U.S. Pat. No. 8,734,809 | AAV CLv-8 See U.S. Pat. No. 8,734,809 |
| SEQ ID NO: 72 | SEQ ID NO: 146 |
| AAV CLv-Dl See U.S. Pat. No. 8,734,809 | AAV CLv-Dl See U.S. Pat. No. 8,734,809 |
| SEQ ID NO: 22 | SEQ ID NO: 96 |
| AAV CLv-D2 See U.S. Pat. No. 8,734,809 | AAV CLv-D2 See U.S. Pat. No. 8,734,809 |
| SEQ ID NO: 23 | SEQ ID NO: 97 |
| AAV CLv-D3 See U.S. Pat. No. 8,734,809 | AAV CLv-D3 See U.S. Pat. No. 8,734,809 |
| SEQ ID NO: 24 | SEQ ID NO: 98 |
| AAV CLv-D4 See U.S. Pat. No. 8,734,809 | AAV CLv-D4 See U.S. Pat. No. 8,734,809 |
| SEQ ID NO: 25 | SEQ ID NO: 99 |
| AAV CLv-D5 See U.S. Pat. No. 8,734,809 | AAV CLv-D5 See U.S. Pat. No. 8,734,809 |
| SEQ ID NO: 26 | SEQ ID NO: 100 |
| AAV CLv-D6 See U.S. Pat. No. 8,734,809 | AAV CLv-D6 See U.S. Pat. No. 8,734,809 |
| SEQ ID NO: 27 | SEQ ID NO: 101 |
| AAV CLv-D7 See U.S. Pat. No. 8,734,809 | AAV CLv-D7 See U.S. Pat. No. 8,734,809 |
| SEQ ID NO: 28 | SEQ ID NO: 102 |
| AAV CLv-D8 See U.S. Pat. No. 8,734,809 | AAV CLv-D8 See U.S. Pat. No. 8,734,809 |
| SEQ ID NO: 29 | SEQ ID NO: 103 AAV CLv-Kl 762 |
| | WO2016065001 SEQ ID NO: 18 |
| AAV CLv-Kl See WO2016065001 SEQ ID NO: 68 | AAV CLv-K3 See WO2016065001 SEQ ID NO: 19 |
| AAV CLv-K3 See WO2016065001 SEQ ID NO: 69 | AAV CLv-K6 See WO2016065001 SEQ ID NO: 20 |
| AAV CLv-K6 See WO2016065001 SEQ ID NO: 70 | AAV CLv-L4 See WO2016065001 SEQ ID NO: 15 |
| AAV CLv-L4 See WO2016065001 SEQ ID NO: 65 | AAV CLv-L5 See WO2016065001 SEQ ID NO: 16 |
| AAV CLv-L5 See WO2016065001 SEQ ID NO: 66 | AAV CLv-L6 See WO2016065001 SEQ ID NO: 17 |
| AAV CLv-L6 See WO2016065001 SEQ ID NO: 67 | AAV CLv-Ml See WO2016065001 SEQ ID NO: 21 |
| AAV CLv-Ml See WO2016065001 SEQ ID NO: 71 | AAV CLv-Mll See WO2016065001 SEQ ID NO: 22 |
| AAV CLv-Ml 1 See WO2016065001 | AAV CLv-M2 See WO2016065001 SEQ ID NO: 23 |
| SEQ ID NO: 72 | |
| AAV CLv-M2 See WO2016065001 SEQ ID NO: 73 | AAV CLv-M5 See WO2016065001 SEQ ID NO: 24 |
| AAV CLv-M5 See WO2016065001 SEQ ID NO: 74 | AAV CLv-M6 See WO2016065001 SEQ ID NO: 25 |
| AAV CLv-M6 See WO2016065001 SEQ ID NO: 75 | AAV CLv-M7 See WO2016065001 SEQ ID NO: 26 |
| AAV CLv-M7 See WO2016065001 SEQ ID NO: 76 | AAV CLv-M8 See WO2016065001 SEQ ID NO: 27 |
| AAV CLv-M8 See WO2016065001 SEQ ID NO: 77 | AAV CLv-M9 See WO2016065001 SEQ ID NO: 28 |
| AAV CLv-M9 See WO2016065001 SEQ ID NO: 78 | AAV CLv-Rl See U.S. Pat. No. 8,734,809 |
| | SEQ ID NO 30 |
| AAV CLv-Rl See U.S. Pat. No. 8,734,809 | AAV CLv-R2 See U.S. Pat. No. 8,734,809 |
| SEQ ID NO 104 | SEQ ID NO 31 |
| AAV CLv-R2 See U.S. Pat. No. 8,734,809 | AAV CLv-R3 See U.S. Pat. No. 8,734,809 |
| SEQ ID NO 105 | SEQ ID NO 32 |
| AAV CLv-R3 See U.S. Pat. No. 8,734,809 | AAV CLv-R4 See U.S. Pat. No. 8,734,809 |
| SEQ ID NO 106 | SEQ ID NO 33 |
| AAV CLv-R4 See U.S. Pat. No. 8,734,809 | AAV CLv-R5 See U.S. Pat. No. 8,734,809 |
| SEQ ID NO 107 | SEQ ID NO 34 |
| AAV CLv-R5 See U.S. Pat. No. 8,734,809 | AAV CLv-R6 See U.S. Pat. No. 8,734,809 |
| SEQ ID NO 108 | SEQ ID NO 35 |
| AAV CLv-R6 See U.S. Pat. No. 8,734,809 | AAV CLv-R7 See U.S. Pat. No. 8,734,809 |
| SEQ ID NO 109 AAV CLv-R7 802 | SEQ ID NO 110 |
| U.S. Pat. No. 8,734,809 SEQ ID NO 36 | |
| AAV CLv-R8 See U.S. Pat. No. 8,734,809 | AAV CLv-R8 See U.S. Pat. No. 8,734,809 |
| SEQ ID NO 37 | SEQ ID NO 111 |
| AAV CLv-R9 See U.S. Pat. No. 8,734,809 | AAV CLv-R9 See U.S. Pat. No. 8,734,809 |
| SEQ ID NO 38 | SEQ ID NO 112 |
| AAV CSp-1 See U.S. Pat. No. 8,734,809 | AAV CSp-1 See U.S. Pat. No. 8,734,809 |
| SEQ ID NO 45 | SEQ ID NO 119 |
| AAV CSp-10 See U.S. Pat. No. 8,734,809 | AAV CSp-10 See U.S. Pat. No. 8,734,809 |
| SEQ ID NO 46 | SEQ ID NO 120 |
| AAV CSp-11 See U.S. Pat. No. 8,734,809 | AAV CSp-11 See U.S. Pat. No. 8,734,809 |
| SEQ ID NO 47 | SEQ ID NO 121 |
| AAV CSp-2 See U.S. Pat. No. 8,734,809 | AAV CSp-2 See U.S. Pat. No. 8,734,809 |
| SEQ ID NO 48 | SEQ ID NO 122 |
| AAV CSp-3 See U.S. Pat. No. 8,734,809 | AAV CSp-3 See U.S. Pat. No. 8,734,809 |
| SEQ ID NO 49 | SEQ ID NO 123 |
| AAV CSp-4 See U.S. Pat. No. 8,734,809 | AAV CSp-4 See U.S. Pat. No. 8,734,809 |
| SEQ ID NO 50 | SEQ ID NO 124 |
| AAV CSp-6 See U.S. Pat. No. 8,734,809 | AAV CSp-6 See U.S. Pat. No. 8,734,809 |
| SEQ ID NO 51 | SEQ ID NO 125 |
| AAV CSp-7 See U.S. Pat. No. 8,734,809 | AAV CSp-7 See U.S. Pat. No. 8,734,809 |
| SEQ ID NO 52 | SEQ ID NO 126 |
| AAV CSp-8 See U.S. Pat. No. 8,734,809 | AAV CSp-8 See U.S. Pat. No. 8,734,809 |
| SEQ ID NO 53 | SEQ ID NO 127 |
| AAV CSp-8.10 See WO2016065001 SEQ ID NO: 38 | AAV CSp-8.10 See WO2016065001 SEQ ID NO: 88 |
| AAV CSp-8.2 See WO2016065001 SEQ ID NO: 39 | AAV CSp-8.2 See WO2016065001 SEQ ID NO: 89 |
| AAV CSp-8.4 See WO2016065001 SEQ ID NO: 40 | AAV CSp-8.4 See WO2016065001 SEQ ID NO: 90 |
| AAV CSp-8.5 See WO2016065001 SEQ ID NO: 41 | AAV CSp-8.5 See WO2016065001 SEQ ID NO: 91 |
| AAV CSp-8.6 See WO2016065001 SEQ ID NO: 42 | AAV CSp-8.6 See WO2016065001 SEQ ID NO: 92 |
| AAV CSp-8.7 See WO2016065001 SEQ ID NO: 43 | AAV CSp-8.7 See WO2016065001 SEQ ID NO: 93 |
| AAV CSp-8.8 See WO2016065001 SEQ ID NO: 44 | AAV CSp-8.8 See WO2016065001 SEQ ID NO: 94 |
| AAV CSp-8.9 See WO2016065001 SEQ ID NO: 45 | AAV CSp-8.9 See WO2016065001 SEQ ID NO: 95 |
| AAV CSp-9 842 U.S. Pat. No. 8,734,809 | AAV CSp-9 See U.S. Pat. No. 8,734,809 |
| SEQ ID NO: 54 | SEQ ID NO: 128 |
| AAV.hu.48R3 See U.S. Pat. No. 8,734,809 | AAV.VR-355 See U.S. Pat. No. 8,734,809 |
| SEQ ID NO: 183 | SEQ ID NO: 181 |
| AAV3B See WO2016065001 SEQ ID NO: 48 | AAV3B See WO2016065001 SEQ ID NO: 98 |
| AAV4 See WO2016065001 SEQ ID NO: 49 | AAV4 See WO2016065001 SEQ ID NO: 99 |
| AAV5 See WO2016065001 SEQ ID NO: 50 | AAV5 See WO2016065001 SEQ ID NO: 100 |
| AAVF1/HSC1 See WO2016049230 SEQ ID NO: 20 | AAVF1/HSC1 See WO2016049230 SEQ ID NO: 2 |
| AAVF11/HSC11 See WO2016049230 SEQ ID NO: 26 | AAVF11/HSC11 See WO2016049230 SEQ ID NO: 4 |
| AAVF12/HSC12 See WO2016049230 SEQ ID NO: 30 | AAVF12/HSC12 See WO2016049230 SEQ ID NO: 12 |
| AAVF13/HSC13 See WO2016049230 SEQ ID NO: 31 | AAVF13/HSC13 See WO2016049230 SEQ ID NO: 14 |
| AAVF14/HSC14 See WO2016049230 SEQ ID NO: 32 | AAVF14/HSC14 See WO2016049230 SEQ ID NO: 15 |
| AAVF15/HSC15 See WO2016049230 SEQ ID NO: 33 | AAVF15/HSC15 See WO2016049230 SEQ ID NO: 16 |
| AAVF16/HSC16 See WO2016049230 SEQ ID NO: 34 | AAVF16/HSC16 See WO2016049230 SEQ ID NO: 17 |
| AAVF17/HSC17 See WO2016049230 SEQ ID NO: 35 | AAVF17/HSC17 See WO2016049230 SEQ ID NO: 13 |
| AAVF2/HSC2 See WO2016049230 SEQ ID NO: 21 | AAVF2/HSC2 See WO2016049230 SEQ ID NO: 3 |
| AAVF3/HSC3 See WO2016049230 SEQ ID NO: 22 | AAVF3/HSC3 See WO2016049230 SEQ ID NO: 5 |
| AAVF4/HSC4 See WO2016049230 SEQ ID NO: 23 | AAVF4/HSC4 See WO2016049230 SEQ ID NO: 6 |
| AAVF5/HSC5 See WO2016049230 SEQ ID NO: 25 | AAVF5/HSC5 See WO2016049230 SEQ ID NO: 11 |
| AAVF6/HSC6 See WO2016049230 SEQ ID NO: 24 | AAVF6/HSC6 See WO2016049230 SEQ ID NO: 7 |
| AAVF7/HSC7 See WO2016049230 SEQ ID NO: 27 | AAVF7/HSC7 See WO2016049230 SEQ ID NO: 8 |
| AAVF8/HSC8 See WO2016049230 SEQ ID NO: 28 | AAVF8/HSC8 See WO2016049230 SEQ ID NO: 9 |
| | AAVF9/HSC9 882 WO2016049230 SEQ ID NO: 29 |
| AAVF9/HSC9 See WO2016049230 SEQ ID NO: 10 | |

The foregoing examples are illustrative of the present invention and are not to be construed as limiting thereof. Although the invention has been described in detail with reference to preferred embodiments, variations and modifications exist within the scope and spirit of the invention as described and defined in the following claims.

We claim:

1. An adeno-associated virus (AAV) capsid protein or derivative thereof, comprising at least a portion of an AAV VP1/VP2 boundary, the capsid protein comprising an amino acid sequence modification at one or more amino acids within the VP1/VP2 boundary that alters permissiveness of a promoter within a cell when the promoter and the capsid protein are present within the cell, and wherein the capsid protein and the promoter are in the context of a recombinant AAV particle.

2. The AAV capsid protein or derivative thereof, of claim 1, wherein the VP1/VP2 boundary corresponds to amino acid 110-170 of AAV9 VP1 (SEQ ID NO:14).

3. The AAV capsid protein or derivative thereof of claim 1 or 2, wherein the amino acid sequence modification alters the electrostatic charge of the VP1/VP2 boundary of the capsid that forms the particle thereby altering the permissiveness of the promoter within the cell.

4. The AAV capsid protein or derivative thereof of any of claims 1-3, wherein the amino acid sequence modification comprises a mutation selected from the group consisting of an insertion, a deletion, a substitution, and combinations thereof.

5. The AAV capsid protein or derivative thereof of any one of claims 1-4, wherein the amino acid sequence modification comprises a modification at one or more amino acids corresponding to from 120 to 150 of AAV9 VP1 (SEQ ID NO:14).

6. The AAV capsid protein or derivative thereof of any one of claims 1-5, wherein the cell is a neuronal cell or a glial cell.

7. The AAV capsid protein or derivative thereof of claim 6, wherein the neuronal cell is a medium spiny neuron, a cholinergic interneuron, or a GABAergic interneuron.

8. The AAV capsid protein or derivative thereof of claim 6, wherein the glial cell is an oligodendrocyte, microglia, or astrocyte.

9. The AAV capsid protein or derivative thereof of any one of claims 1-8, wherein the promoter is a ubiquitous promoter.

10. The AAV capsid protein or derivative thereof of any one of claims 1-8, wherein the promoter is a tissue specific promoter.

11. The AAV capsid protein or derivative thereof of any one of claims 1-11, wherein the amino acid sequence modification is an insertion of from 1-20 amino acid residues, from 4-16 amino acid residues, from 6-14 amino acid residues, or from 3-200 amino acid residues.

12. The AAV capsid protein or derivative thereof of any one of claims 1-11, wherein the amino acid sequence modification is an insertion of 3 or more amino acid residues.

13. The AAV capsid protein or derivative thereof of any one of claims 1-12, wherein the amino acid sequence modification preserves nuclear localization signals and phospholipase domains present in the capsid protein.

14. The AAV capsid protein or derivative thereof of any one of claims 1-13, wherein the amino acid sequence modification is a modification to the amino acid sequence of VP2.

15. The AAV capsid protein or derivative thereof of any one of claims 1-14, wherein the amino acid sequence modification is an insertion between amino acids corresponding to 137 and 138 of AAV9 capsid protein (SEQ ID NO:14), or between amino acids corresponding to 138 and 139 of AAV9 capsid protein (SEQ ID NO:14).

16. The AAV capsid protein or derivative thereof of any one of claims 1-15, wherein the amino acid sequence modification increases expression from the promoter in oligodendrocytes and/or decreases expression from the promoter in neurons.

17. The AAV capsid protein or derivative thereof of any one of claims 1-16, wherein the amino acid sequence modification is an insertion of residues having an overall negative charge.

18. The AAV capsid protein or derivative thereof of claim 17, wherein the amino acid sequence modification is an insertion of 2 or more glutamate residues.

19. The AAV capsid protein or derivative thereof of claim 18, wherein the amino acid sequence modification is an insertion of 6 glutamate residues.

20. The AAV capsid protein or derivative thereof of any one of claims 1-16, wherein the amino acid sequence modification is an insertion of residues having an overall positive charge.

21. The AAV capsid protein or derivative thereof of claim 20, wherein the amino acid sequence modification is an insertion of substance P peptide.

22. The AAV capsid protein or derivative thereof of any one of any one of claims 1-15, wherein the amino acid sequence modification increases expression from the promoter in neurons and/or decreases expression from the promoter in oligodendrocytes.

23. The AAV capsid protein or derivative thereof of any one of claims 1-22, wherein the amino acid sequence modification is an insertion of residues having an overall neutral charge.

24. The AAV capsid protein or derivative thereof of claim 23, wherein the amino acid sequence modification is an insertion of 2 or more alanine residues.

25. The AAV capsid protein or derivative thereof of claim 24, wherein the amino acid sequence modification is an insertion of 6 alanine residues.

26. The AAV capsid protein or derivative thereof of any of claims 1-25, wherein the capsid protein is from a serotype selected from the group consisting of AAV1, AAV2, AAV3, AAV4, AAV5, AAV6, AAV7, AAV8, AAV9, AAV10, AAV11, AAV12, and AAV13.

27. The AAV capsid protein or derivative thereof of claim 26, wherein the capsid protein is from AAV2.

28. The AAV capsid protein or derivative thereof of claim 26, wherein the capsid protein is from AAV8.

29. The AAV capsid protein or derivative thereof of claim 26, wherein the capsid protein is from AAV9.

30. The AAV capsid protein or derivative thereof of any of claims 1-26, wherein the rAAV particle is selected from the group consisting of AAV1, AAV2, AAV3, AAV4, AAV5, AAV6, AAV7, AAV8, AAV9, AAV10, AAV11, AAV12, and AAV13.

31. The AAV capsid protein or derivative thereof of claim 30, wherein the rAAV particle is AAV2.

32. The AAV capsid protein or derivative thereof of claim 30, wherein the rAAV particle is AAV8.

33. The AAV capsid protein or derivative thereof of claim 30, wherein the rAAV particle is AAV9.

34. A method for altering expression of a transgene operably linked to a promoter and delivered to a cell by a rAAV vector comprising modifying the amino acid sequence of at least one amino acid within the VP1/VP2 boundary of a capsid protein or derivative thereof of the rAAV vector, wherein the amino acid sequence modification alters the permissiveness of the promoter within the cell.

35. The method of claim 34, wherein the VP1/VP2 boundary corresponds to amino acid 110-170 of AAV9 (SEQ ID NO:14).

36. The method of claim 34 or 35, wherein the amino acid sequence modification alters the electrostatic charge of the VP1/VP2 boundary of the capsid that forms the particle thereby altering the permissiveness of the promoter within the cell.

37. The method of any one of claims 34-36, wherein the amino acid sequence modification comprises a mutation selected from the group consisting of an insertion, a deletion, a substitution, and combinations thereof.

38. The method of any one of claims 34-37, wherein the amino acid sequence modification comprises a modification at one or more amino acids corresponding to from 120 to 150 of AAV9 VP1 (SEQ ID NO:14).

39. The method of any one of claims 34-38, wherein the cell is a neuronal cell or a glial cell.

40. The method of claim 39, wherein the neuronal cell is a medium spiny neuron, a cholinergic interneuron, or a GABAergic interneuron.

41. The method of claim 39, wherein the glial cell is an oligodendrocyte, microglia, or astrocyte.

42. The method of any one of claims 34-41, wherein the promoter is a ubiquitous promoter.

43. The method of any one of claims 34-41, wherein the promoter is a tissue specific promoter.

44. The method of any one of claims 34-41, wherein the promoter is the CBA promoter.

45. The method of any one of claims 34-41, wherein the promoter is the CBh promoter.

46. The method of any one of claims 34-41, wherein the promoter is the JeTI promoter.

47. The method of any one of claims 34-41, wherein the promoter is the synapsin promoter.

48. The method of any one of claims 34-41, wherein the promoter is cytomegalovirus (CMV) immediate-early enhancer and chicken beta-actin (CAG), cytomegalovirus (CMV), CMV/chicken β-actin (CMV/β-actin), elongation factor 1α (EF1α), phosphoglycerate kinase, ubiquitin C (UbC), CB, CBA, and CBh, JeTI, human synapsin promoter (hSYN1), cytomegalovirus (CMV) promoter, or a CB7 promoter, the human β-actin promoter, the human elongation factor-1 α promoter, the simian virus 40 promoter, and the herpes simplex virus thymidine kinase promoter, dihydrofolate reductase promoter, the phosphoglycerol kinase (PGK) promoter, the rhodopsin kinase promoter, the rhodopsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter, the ornithine transcarbamylase (OTC) promoter the alpha 1-antitrypsin (AAT) promoter, the albumin promoter, hepatitis B virus core promoter, thyroxin binding globulin (TGB) promoter and the LSP1 promoter, skeletal β-actin promoter, myosin light chain 2A, dystrophin, muscle creatine kinase, liver (albumin) promoter, hepatitis B virus core promoter, alpha-fetoprotein (AFP), bone osteocalcin, bone sialoprotein, lymphocytes (CD2), immunoglobulin heavy chain, T cell receptor chain, neuron-specific enolase (NSE) promoter, neurofilament light-chain gene promoter, the neuron-specific vgf gene promoter, Synapsinl, CaMKII alpha, GAD67, GAD65, VGAT, SP-B promoter, ICAM promoter, IFN beta promoter, CD45 promoter, OG-2 promoter, NSE promoter, MSK promoter, JeTI, SPc5-12, 2R5Sc5-12, dMCK, tMCK, SP1 element, synthetic cold-shock responsive promoter (calcyclin), sps5, sps8, synthetic promoter inducible under acidic conditions derived from the YGP1 gene, synthetic promoter inducible under acidic conditions derived from the CCW14 gene, YGP1pr, tTA, tTA-dependent promoter, cumate inducible promoter, 4-hydroxytamoxifen (OHT)-inducible promoter, metallothionein-inducible promoter, rapamycin-inducible promoter, or Rheoswitch® inducible promoter.

49. The method of any one of claims 34-48, wherein the amino acid sequence modification is an insertion of from 1-20 amino acid residues, from 4-16 amino acid residues, from 6-14 amino acid residues, or from 3-200 amino acid residues.

50. The method of any one of claims 34-49, wherein the amino acid sequence modification is an insertion of 3 or more amino acid residues.

51. The method of any one of claims 34-50, wherein the amino acid sequence modification preserves nuclear localization signals and phospholipase domains present in the capsid protein.

52. The method of any one of claims 34-51, wherein the amino acid sequence modification is a modification to the amino acid sequence of VP2.

53. The method of any one of claims 34-52, wherein the amino acid sequence modification is an insertion between amino acids corresponding to 137 and 138 of AAV9 (SEQ ID NO:14), or between amino acids corresponding to 138 and 139 of AAV9 (SEQ ID NO:14).

54. The method of any one of claims 34-53, wherein the amino acid sequence modification increases expression from the promoter in oligodendrocytes and/or decreases expression from the promoter in neurons.

55. The method of any one of claims 34-54, wherein the amino acid sequence modification is an insertion of residues having an overall negative charge.

56. The method of claim 55, wherein the amino acid sequence modification is an insertion of 2 or more glutamate residues.

57. The method of claim 56, wherein the amino acid sequence modification is an insertion of 6 glutamate residues.

58. The method of any one of claims 34-54, wherein the amino acid sequence modification is an insertion of residues having an overall positive charge.

59. The method of claim 34-53, wherein the amino acid sequence modification is an insertion of substance P peptide.

60. The method of any one of any one of claims 34-53, wherein the amino acid sequence modification increases expression from the promoter in neurons and/or decreases expression of from the promoter in oligodendrocytes.

61. The method of any one of claims 34-53, wherein the amino acid sequence modification is an insertion of residues having an overall neutral charge.

62. The method of claim 61, wherein the amino acid sequence modification is an insertion of 2 or more alanine residues.

63. The method of claim 62, wherein the amino acid sequence modification is an insertion of 6 alanine residues.

64. The method of any of claims 34-63, wherein the capsid protein is from a serotype selected from the group consisting of AAV1, AAV2, AAV3, AAV4, AAV5, AAV6, AAV7, AAV8, AAV9, AAV10, AAV11, AAV12, and AAV13.

65. The AAV capsid protein or derivative thereof of claim 64, wherein the capsid protein is from AAV2.

66. The AAV capsid protein or derivative thereof of claim 64, wherein the capsid protein is from AAV8.

67. The AAV capsid protein or derivative thereof of claim 64, wherein the capsid protein is from AAV9.

68. The method of any of claims 34-67, wherein the rAAV particle is selected from the group consisting of AAV1, AAV2, AAV3, AAV4, AAV5, AAV6, AAV7, AAV8, AAV9, AAV10, AAV11, AAV12, and AAV13.

69. The AAV capsid protein or derivative thereof of claim 68, wherein the rAAV particle is AAV2.

70. The AAV capsid protein or derivative thereof of claim 68, wherein the rAAV particle is AAV8.

71. The AAV capsid protein or derivative thereof of claim 68, wherein the rAAV particle is AAV9.

72. A nucleic acid encoding the AAV capsid protein or derivative thereof of any one of claims 1-33.

73. The nucleic acid of claim 72, wherein the nucleic acid is comprised within a vector.

74. The nucleic acid of claim 73, wherein the vector is a plasmid, phage, viral vector, bacterial artificial chromosome, or yeast artificial chromosome.

75. The nucleic acid of claim 74, wherein the viral vector is an AAV vector.

76. The nucleic acid of claim 75, wherein the nucleic acid further comprises an AAV rep coding sequence.

77. The nucleic acid of any one of claims 73-76, wherein the vector further comprises a promoter operably linked to a heterologous polynucleotide.

78. The nucleic acid of claim 77, wherein the promoter is a synthetic promoter.

79. The nucleic acid of claim 77, wherein the promoter is the CBA promoter.

80. The nucleic acid of claim 77, wherein the promoter is the CBh promoter.

81. The nucleic acid of claim 77, wherein the promoter is the JeTI promoter.

82. The nucleic acid of claim 77, wherein the promoter is the synapsin promoter.

83. The nucleic acid of claim 77, wherein the promoter is cytomegalovirus (CMV) immediate-early enhancer and chicken beta-actin (CAG), cytomegalovirus (CMV), CMV/chicken β-actin (CMV/β-actin), elongation factor 1α (EF1α), phosphoglycerate kinase, ubiquitin C (UbC), CB, CBA, and CBh, JeTI, human synapsin promoter (hSYN1), cytomegalovirus (CMV) promoter, or a CB7 promoter, the human β-actin promoter, the human elongation factor-1 α promoter, the simian virus 40 promoter, and the herpes simplex virus thymidine kinase promoter, dihydrofolate reductase promoter, the phosphoglycerol kinase (PGK) promoter, the rhodopsin kinase promoter, the rhodopsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter, the ornithine transcarbamylase (OTC) promoter the alpha 1-antitrypsin (AAT) promoter, the albumin promoter, hepatitis B virus core promoter, thyroxin binding globulin (TGB) promoter and the LSP1 promoter, skeletal β-actin promoter, myosin light chain 2A, dystrophin, muscle creatine kinase, liver (albumin) promoter, hepatitis B virus core promoter, alpha-fetoprotein (AFP), bone osteocalcin, bone sialoprotein, lymphocytes (CD2), immunoglobulin heavy chain, T cell receptor chain, neuron-specific enolase (NSE) promoter, neurofilament light-chain gene promoter, the neuron-specific vgf gene promoter, Synapsinl, CaMKII alpha, GAD67, GAD65, VGAT, SP-B promoter, ICAM promoter, IFN beta promoter, CD45 promoter, OG-2 promoter, NSE promoter, MSK promoter, JeTI, SPc5-12, 2R5Sc5-12, dMCK, tMCK, SP1 element, synthetic cold-shock responsive promoter (calcyclin), sps5, sps8, synthetic promoter inducible under acidic conditions derived from the YGP1 gene, synthetic promoter inducible under acidic conditions derived from the CCW14 gene, YGP1pr, tTA, tTA-dependent promoter, cumate inducible promoter, 4-hydroxytamoxifen (OHT)-inducible promoter, metallothionein-inducible promoter, rapamycin-inducible promoter, or Rheoswitch® inducible promoter.

84. A cell in vitro comprising the nucleic acid of any one of claims 72-83 stably incorporated into the genome.

85. A virus particle comprising the nucleic acid of any one of claims 72-83.

86. The virus particle of claim 85, wherein the virus particle is an AAV particle, an adenovirus particle, a herpesvirus particle, or a baculovirus particle.

87. An AAV particle comprising:
an AAV vector genome; and
the AAV capsid protein or derivative thereof of any one of claims 1-33, wherein the AAV capsid protein or derivative thereof encapsidates the AAV vector genome.

88. The AAV particle of claim 87, wherein the AAV vector genome comprises a promoter operably linked to a heterologous nucleic acid.

89. The AAV particle of claim 88, wherein the promoter is a synthetic promoter.

90. The AAV particle of claim 88, wherein the promoter is the CBA promoter.

91. The AAV particle of claim 88, wherein the promoter is the CBh promoter.

92. The AAV particle of claim 88, wherein the promoter is the JeTI promoter.

93. The AAV particle of claim 88, wherein the promoter is the synapsin promoter.

94. The AAV particle of claim 88, wherein the promoter is cytomegalovirus (CMV) immediate-early enhancer and chicken beta-actin (CAG), cytomegalovirus (CMV), CMV/chicken β-actin (CMV/β-actin), elongation factor 1α (EF1α), phosphoglycerate kinase, ubiquitin C (UbC), CB, CBA, and CBh, Jell, human synapsin promoter (hSYN1), cytomegalovirus (CMV) promoter, or a CB7 promoter, the human β-actin promoter, the human elongation factor-1 α promoter, the simian virus 40 promoter, and the herpes simplex virus thymidine kinase promoter, dihydrofolate reductase promoter, the phosphoglycerol kinase (PGK) promoter, the rhodopsin kinase promoter, the rhodopsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter, the ornithine transcarbamylase (OTC) promoter the alpha 1-antitrypsin (AAT) promoter, the albumin promoter, hepatitis B virus core promoter, thyroxin binding globulin (TGB) promoter and the LSP1 promoter, skeletal β-actin promoter, myosin light chain 2A, dystrophin, muscle creatine kinase, liver (albumin) promoter, hepatitis B virus core promoter, alpha-fetoprotein (AFP), bone osteocalcin, bone sialoprotein, lymphocytes (CD2), immunoglobulin heavy chain, T cell receptor chain, neuron-specific enolase (NSE) promoter, neurofilament light-chain gene promoter, the neuron-specific vgf gene promoter, Synapsinl, CaMKII alpha, GAD67, GAD65, VGAT, SP-B promoter, ICAM promoter, IFN beta promoter, CD45 promoter, OG-2 promoter, NSE promoter, MSK promoter, JeTI, SPc5-12, 2R5Sc5-12, dMCK, tMCK, SP1 element, synthetic cold-shock responsive promoter (calcyclin), sps5, sps8, synthetic promoter inducible under acidic conditions derived from the YGP1 gene, synthetic promoter inducible under acidic conditions derived from the CCW14 gene, YGP1pr, tTA, tTA-dependent promoter, cumate inducible promoter, 4-hydroxytamoxifen (OHT)-inducible promoter, metallothionein-inducible promoter, rapamycin-inducible promoter, or Rheoswitch® inducible promoter.

95. The AAV particle of any one of claims 88-94, wherein the heterologous nucleic acid encodes an antisense RNA, microRNA, or RNAi.

96. The AAV particle of any one of claims 88-94, wherein the heterologous nucleic acid encodes a polypeptide.

97. The AAV particle of claim 96, wherein the heterologous nucleic acid encodes a therapeutic polypeptide.

98. The AAV particle of claim 96, wherein the heterologous nucleic acid encodes a reporter protein.

99. A method of producing a recombinant AAV particle comprising an AAV capsid, the method comprising:
providing a cell in vitro with a nucleic acid according to any one of claims 72-83, an AAV rep coding sequence, an AAV vector genome comprising a promoter operably linked to a heterologous nucleic acid, and helper functions for generating a productive AAV infection; and
allowing assembly of the recombinant AAV particle comprising the AAV capsid and encapsidating the AAV vector genome.

100. An AAV particle produced by the method of claim 99.

101. A pharmaceutical formulation comprising the AAV capsid protein or derivative thereof of any one of claims 1-33, the nucleic acid of any one of claims 72-83, the virus particle of claim 85 or 86, or the AAV particle of any one of claim 87-98 or 100 in a pharmaceutically acceptable carrier.

102. A method of delivering a nucleic acid of interest to a cell, the method comprising contacting the cell with the AAV particle of any one of claim 87-98 or 100.

103. The method of claim 102, wherein the cell is a central nervous system (CNS) cell.

104. A method of delivering a nucleic acid of interest to a cell in a mammalian subject, the method comprising:
administering an effective amount of the AAV particle of any one of claim 87-98 or 100 or the pharmaceutical formulation of claim 82 to a mammalian subject, thereby delivering the nucleic acid of interest to a cell in the mammalian subject.

105. The method of claim 104, wherein the cell is a central nervous system cell.

106. The method of claim 104 or 105, wherein the mammalian subject is a human subject.

107. The method of any one of claims 104-106, wherein the AAV particle is delivered to the CNS.

108. The method of claim 107, wherein the AAV particle is delivered directly to the CNS by intrathecal, intracerebral, intraventricular, intranasal, intra-aural, intra-ocular, or peri-ocular delivery, or any combination thereof.

109. A method of treating a disorder in a mammalian subject in need thereof, wherein the disorder is treatable by expressing a therapeutic product in cells of the subject, the method comprising administering a therapeutically effective amount of the AAV particle of any one of claim 87-98 or 100 or the pharmaceutical formulation of claim 80 to a mammalian subject, wherein the product is expressed, thereby treating the disorder.

110. The method of claim 109, wherein the cells of the subject are in the CNS.

111. A method of altering expression of a heterologous polynucleotide present in an AAV vector in cells of a subject, comprising preparing the AAV vector with the AAV capsid protein or derivative thereof of any one of claims 1-33.

112. The method of claim 111, where the cells are CNS cells.


SEQUENCE LISTING (19 sequences)

SEQ ID NO: 1 - protein, 60 aa, adeno-associated virus 2
GGNLGRAVFQAKKRVLEPLGLVEEPVKTAPGKKRPVEHSPVEPDSSSGTGKAGQQPARKR

SEQ ID NO: 2 - protein, 61 aa, adeno-associated virus 9
GGNLGRAVFQAKKRVLEPLGLVEEAAKTAPGKKRPVEPSPQRSPDSSTGIGKKGQQPAKKR

SEQ ID NO: 3 - DNA, 6 bp, Artificial
  Insert
aaaaaa

SEQ ID NO: 4 - protein, 6 aa, Artificial
  Insert
EEEEEE

SEQ ID NO: 5 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 6 - protein, 735 aa, adeno-associated virus 2 [sequence omitted]

SEQ ID NO: 7 - protein, 736 aa, adeno-associated virus 3a
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 8 - protein, 736 aa, adeno-associated virus 3b
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 9 - protein, 734 aa, adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 10 - protein, 724 aa, adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - protein, 736 aa, adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 12 - protein, 737 aa, adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 13 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 14 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 738 aa, adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 16 - protein, 733 aa, adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 17 - protein, 742 aa, adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 18 - protein, 733 aa, adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 19 - protein, 11 aa, Homo sapiens
RPKPQQFFGLM